(19) United States
(12) Patent Application Publication
(10) Pub. No.: US 2025/0066351 A1
(43) Pub. Date: Feb. 27, 2025

(54) PYRAZOLO[1,5-A]PYRIDIN-2,3-YL AMIDES AS KV7 CHANNEL ACTIVATORS

(71) Applicant: Biohaven Therapeutics Ltd., New Haven, CT (US)

(72) Inventors: Lynn RESNICK, Pittsburgh, PA (US); Justin K. BELARDI, Pittsburgh, PA (US); Charles A. FLENTGE, Mars, PA (US); David A. MARESKA, McMurray, PA (US); George T. TOPALOV, Pittsburgh, PA (US); Steven A. BOYD, Mars, CT (US); James S. HALE, Glenshaw, PA (US)

(21) Appl. No.: 18/726,772

(22) PCT Filed: Jan. 6, 2023
(86) PCT No.: PCT/US2023/010295

§ 371 Date: Jul. 3, 2024

Related U.S. Application Data
(60) Provisional application No. 63/297,295, filed on Jan. 7, 2022.

(51) Int. Cl. C07D 471/04; A61K 31/437; A61K 31/444

(52) U.S. Cl. CPC C07D 471/04; A61K 31/437; A61K 31/444

(57) ABSTRACT

Compounds such as those represented by Formula I can be potent for the Kv7.2/7.3 heteromultimer. These compounds may be used to treat a number of medical conditions, and may be included in dosage forms, pharmaceutical compositions, or medicaments.

### CROSS-REFERENCE TO RELATED APPLICATIONS

This application claims priority to and the benefit of U.S. Provisional Application No. 63/297,295 filed Jan. 7, 2022, the content of which is incorporated herein in its entirety by reference.

### GOVERNMENT INTERESTS

This invention was made with United States Government support under Grant No. U44NS093160 awarded by the National Institute of Neurological Disorders and Stroke of the National Institutes of Health. The United States Government has certain rights in the invention.

### SUMMARY

Potassium (K⁺) channels, present on the plasma membranes of most cell types, are the most diverse class of all ion channels and are associated with a wide range of physiological functions including the regulation of the electrical properties of excitable cells. The primary pore-forming (a) subunits of these highly selective cation channels are divided into three primary structural classes based on the number of transmembrane (TM)-spanning regions and pore (P) regions: currently there are known to be 6TM/1P, 2TM/1P and 4TM/2P K⁺ channels. The Kv7 genes (originally termed KCNQ, a name assigned by the HUGO Gene Nomenclature Committee (HGNC)) were assigned to a subfamily of voltage-gated K⁺ channels by the International Union of Pharmacology (IUPHAR). The Kv7 subfamily consists of five homologous pore-forming a subunits, Kv7.1-7.5, that have a structure typical of voltage-gated K⁺ channels with 6TM-spanning regions (S1-S6) flanked by intracellular N-terminal and C-terminal domains, a typical voltage-sensor domain located in S4 comprised of alternating positively-charged residues and a single P region between S5 and S6 of each subunit. The channels are formed as tetramers of the primary a subunits, either as homotetramers or heterotetramers. Neurons are known to express Kv7 channels comprised of Kv7.2-7.5α subunits. Some of these gene products may be exclusively neuronal while others, such as Kv7.4 and Kv7.5, can be found in other tissues such as smooth and skeletal muscle.

Native M-channels, and the corresponding macroscopic M-current, were first characterized in amphibian sympathetic neurons. M-channels were notable because they were slowly activating and non-inactivating, active at membrane potentials at or near the resting membrane potential of neurons and muscarinic cholinergic agonists produced a reduction in the M-current, demonstrating a direct and inhibitory link between G-protein coupled receptors (GPCRs) and a physiological K⁺ current. It was not until the cloning of this subfamily of genes that the pharmacological and biophysical identity was established between Kv7.2/7.3 (and likely Kv7.5/7.3) heteromultimers and the elusive M-channel, providing significant new evidence for their importance in neuronal regulation.

The distributions of these channels, both regionally and developmentally, as well as their biophysical characteristics, support their role in providing enduring resistance to depolarizing excitatory influences. Under physiological conditions, as was demonstrated with native M-channels, they can be very effective at regulating the sub-threshold excitability of certain neuronal populations with significant roles in regulating the frequency and ultimately the pattern of action potential discharge in many types of neurons. Their importance in neuronal regulation was punctuated by the discovery that neuronal Kv7 mutations lead to benign familial neonatal convulsions (BFNC) indicating that reduction or removal of the influence of Kv7.2 and Kv7.3 channels can dramatically alter neuronal excitability. Mutation analyses demonstrated their involvement in BFNC and suggested their utility as targets for anti-epileptic drugs (AEDs).

Unlike established pharmacological terminology for GPCRs, the mode of action of K⁺ channel modulators, in particular compounds that activate the channel, is still being refined. The application of voltage-clamp techniques to the study of ion channel pharmacology enabled detailed biophysical studies on either whole-cell currents or single channels, allowing some characterization of the nature of compound-channel interactions but not preventing ongoing confusion around the terminology. The term opener or activator is commonly used throughout the literature but does not adequately describe the mode of action of all these ‘positive modulator’ compounds. In general, openers or activators are expected to increase the open probability of the channel or increase macroscopic current amplitude, but this nomenclature is really too simplistic. For example, retigabine, the first publicly disclosed Kv7 opener, has a complex and interesting profile in that it has inhibitory activity at higher membrane potentials. Neuronal Kv7 channel openers may work in concert with the activity of a channel over the ‘normal’ activation-voltage range and enhance currents without significantly affecting the activation threshold while others can significantly alter the activation threshold. In addition, some openers appear to remove the voltage-dependence of activation entirely. Whether these effects represent some continuum is currently unclear since the effects are often concentration-dependent. Clearly, the modes of interaction of compounds that can increase channel current are complex and in most cases not well understood and the implications of these profiles on neuronal responsiveness and systems physiology are also unclear. Retigabine is modestly potent, not highly specific, but it is a very effective opener of Kv7.2, Kv7.5 and heteromultimeric Kv7 channels. Its effects are characterized by a significant increase in channel current over a narrow voltage range. As mentioned above, at more positive voltages the opener is less effective and under some conditions channel current significantly decreases at more positive voltages relative to control currents (this ‘crossover’ voltage-dependence of opener action is a characteristic of many neuronal Kv7 channel openers). This effect is also concentration-dependent and is more pronounced at higher concentrations.

Provided herein are compounds that can be potent and/or at least biased for the Kv7.2/7.3 heteromultimer over the Kv7.4 homomultimer. These compounds may have reduced untoward side effects as compared to retigabine.

### DETAILED DESCRIPTION

Before the present compositions and methods are described, it is to be understood that any invention is not limited to the particular processes, compositions, or methodologies described, as these may vary. Moreover, the processes, compositions, and methodologies described in particular embodiments are interchangeable. Therefore, for example, a composition, dosage regimen, route of administration, and so on described in a particular embodiment may be used in any of the methods described in other particular embodiments. It is also to be understood that the terminology used in the description is for the purpose of describing the particular versions or embodiments only, and is not intended to limit the scope of the present invention, which will be limited only by the appended claims. Unless clearly defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention. It must be noted that, as used herein, and in the appended claims, the singular forms “a”, “an”, and “the” include plural reference unless the context clearly dictates otherwise.

As used herein, the term “about” means plus or minus 10% of the numerical value of the number with which it is being used. Therefore, about 50% means in the range of 45%-55%. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

“Administering”, when used in conjunction with a therapeutic, means to administer a therapeutic directly into or onto a target tissue or to administer a therapeutic to a subject whereby the therapeutic positively impacts the tissue to which it is targeted. “Administering” a composition may be accomplished by oral administration, injection, infusion, absorption or by any method in combination with other known techniques. “Administering” may include the act of self-administration or administration by another person such as a healthcare provider or a device.

As used herein, the terms “comprising,” “comprise,” “comprises,” and “comprised” are inclusive or open-ended and do not exclude additional, unrecited elements or method steps.

As used herein, the term “consists of” or “consisting of” means that the composition or method includes only the elements, steps, or ingredients specifically recited in the particular embodiment or claim.

As used herein, the term “consisting essentially of” or “consists essentially of” means that the composition or method includes only the specified materials or steps and those that do not materially affect the basic and novel characteristics of the claimed invention.

In each of the embodiments disclosed herein, the compositions and methods may be utilized with or on a subject in need of such treatment, which may also be referred to as “in need thereof.” As used herein, the phrase “in need thereof” means that the subject has been identified as having a need for the particular method or treatment and that the treatment has been given to the subject for that particular purpose.

As used herein, the term “patient” and “subject” are interchangeable and may be taken to mean any living organism, which may be treated with compounds of the present invention. As such, the terms “patient” and “subject” may include, but is not limited to, any non-human mammal, primate or human. In some embodiments, the “patient” or “subject” is an adult, child, infant, or fetus. In some embodiments, the “patient” or “subject” is a human. In some embodiments, the “patient” or “subject” is a mammal, such as mice, rats, other rodents, rabbits, dogs, cats, swine, cattle, sheep, horses, primates, or humans.

The terms “therapeutically effective amount” or “therapeutic dose” is used herein are interchangeable and may refer to the amount of an active agent or pharmaceutical compound or composition that elicits a clinical, biological or medicinal response in a tissue, system, animal, individual or human that is being sought by a researcher, veterinarian, medical doctor or other clinical professional. A clinical, biological or medical response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experience or exhibited by the individual.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to alleviating the symptoms associated with the specific disorder, disease or condition. In some embodiments, the term refers to alleviating the symptoms associated with the specific disorder, disease or condition. In some embodiments, the term refers to restoring function which was impaired or lost due to a specific disorder, disorder or condition.

“Pharmaceutically acceptable salt” is meant to indicate those salts which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol. 6, 1-19, describes pharmaceutically acceptable salts in detail. A pharmaceutical acceptable “salt” is any acid addition salt, preferably a pharmaceutically acceptable acid addition salt, including, but not limited to, halogenic acid salts such as hydrobromic, hydrochloric, hydrofloric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethane sulfonic, ethanesulfonic, benzenesulfonic or p-toluenesufonic, acetic, malic, fumaric, succinic, citric, benzonic gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di-hydrohalogic, di-sulfuric, di-phosphoric or di-organic acid salt. In all cases, the acid addition salt is used as an achiral reagent which is not selected on the basis of any expected or known preference for the interaction with or precipitation of a specific optical isomer of the products of this disclosure.

Unless otherwise indicated, when a compound or chemical structural feature such as pyrazolo[1,5-a]pyridin-2-ylcarbamoyl is referred to as being “optionally substituted,” it includes a feature that has no substituents (i.e. unsubstituted), or a feature that is “substituted,” meaning that the feature has one or more substituents. The term “substituent” has the broadest meaning known to one of ordinary skill in the art and includes a moiety that replaces one or more hydrogen atoms attached to a parent compound or structural feature. In some embodiments, a substituent may be an ordinary organic moiety known in the art, which may have a molecular weight (e.g. the sum of the atomic masses of the atoms of the substituent) of 15 Da to 50 Da, 15 Da to 100 Da, 15 Da to 150 Da, 15 Da to 200 Da, 15 Da to 300 Da, or 15 Da to 500 Da. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, Si, F, Cl, Br, or I; provided that the substituent includes one C, N, O, S, Si, F, Cl, Br, or I atom. In some embodiments, a substituent can be a moiety having a molecular weight of 15 Da to 300 Da, 15 Da to 200 Da, 15 Da to 150 Da, or 15 Da to 100 Da; and consisting of 1 to 5 chemical elements, wherein the chemical elements are independently C, H, O, N, S, F, Cl, or Br.

Examples of substituents include, but are not limited to, hydrocarbyl, such as alkyl, alkenyl, alkynyl; heteroalkyl, including any alkyl wherein one or more heteroatoms replaces: one or more carbon atoms and possibly some hydrogen atoms accompanying the carbon atoms (e.g. N replaces CH, O replaces CH₂, Cl replaces CH₃, etc.), such as alkoxy, alkylthio, haloalkyl, haloalkoxy, amino, etc.; heteroalkenyl, including any alkenyl wherein one or more heteroatoms replaces: one or more carbon atoms and possibly some hydrogen atoms accompanying the carbon atoms, such as acyl, acyloxy, thiocarbonyl, alkylcarboxylate, O-carbamyl, N-carbamyl, O-thiocarbamyl, N-thiocarbamyl, C-amido, N-amido, sulfinyl, isocyanato, isothiocyanato, etc; heteroalkynyl, including any alkynyl wherein one or more heteroatoms replaces: one or more carbon atoms and possibly some hydrogen atoms accompanying the carbon atoms, such as cyano, thiocyanato, cyanato; aryl; heteroaryl; hydroxy; aryloxy; thiol; halo; S-sulfonamido; N-sulfonamido; nitro, silyl; sulfonyl; trihalomethanesulfonyl; trihalomethanesulfonamido; etc.

For convenience, the term “molecular weight” is used with respect to a moiety or part of a molecule to indicate the sum of the atomic masses of the atoms in the moiety or part of a molecule, even though it may not be a complete molecule.

The structures associated with some of the chemical names referred to herein are depicted below. These structures may be unsubstituted, as shown below, or a substituent may independently be in any position normally occupied by a hydrogen atom when the structure is unsubstituted. Unless a point of attachment is indicated by— [symbol] , attachment may occur at any position normally occupied by a hydrogen atom.

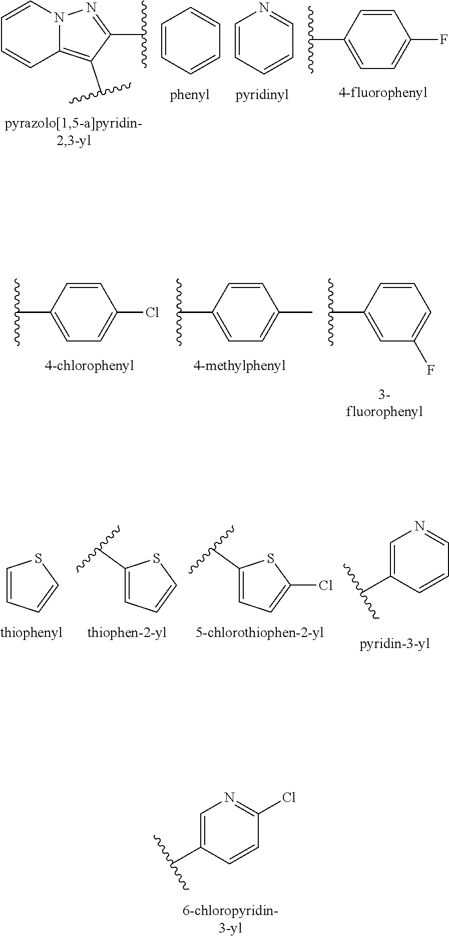

As used herein, the term “alkyl” has the broadest meaning generally understood in the art and may include a moiety composed of carbon and hydrogen containing no double or triple bonds. Alkyl may be linear alkyl, branched alkyl, cycloalkyl, or a combination thereof and in some embodiments, may contain from one to thirty-five carbon atoms. In some embodiments, alkyl may include C₁₋₁₀ linear alkyl, such as methyl (—CH₃), methylene (—CH₂—), ethyl (—CH₂CH₃), ethylene (—C₂H₄—), propylene (—C₃CH₆—), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₁₀ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), C₇H₁₅ (e.g. heptyl isomers), etc.; C₃₋₁₀ cycloalkyl, such as C₃H₅ (e.g. cyclopropyl), C₄H₇ (e.g. cyclobutyl isomers such as cyclobutyl, methylcyclopropyl, etc.), C₅H₉ (e.g. cyclopentyl isomers such as cyclopentyl, methylcyclobutyl, dimethylcyclopropyl, etc.) C₆H₁₁ (e.g. cyclohexyl isomers), C₇H₁₃ (e.g. cycloheptyl isomers), etc.; and the like.

With respect to an optionally substituted moiety such as optionally substituted alkyl, a phrase such as “optionally substituted C₁₋₁₂ alkyl” refers to a C₁₋₁₂ alkyl that may be unsubstituted, or may have 1 or more substituents, and does not limit the number of carbon atoms in any substituent. Thus, for example, CH₂(CH₂)₁₁OCH₃ is optionally substituted C₁₋₁₂ alkyl because the parent alkyl group has 12 carbon atoms. A phrase such as “C₁₋₁₂ optionally substituted alkyl” refers to unsubstituted C₁₋₁₂ alkyl, or substituted alkyl wherein the alkyl parent and all substituents together have from 1-12 carbon atoms. For example, CH₂CH₂OCH₃ is C₁₋₁₂ optionally substituted alkyl because the alkyl group (e.g. ethyl) and the substituent (e.g. methoxy) together contain 3 carbon atoms. Similar conventions may be applied to other optionally substituted moieties such as aryl and heterocyclyl.

Substituents on alkyl may be the same as those described generally above. In some embodiments, substituents on alkyl are independently selected from F, Cl, Br, I, CN, CO₂H, —O-alkyl, ester groups, acyl, amine groups, amide groups, phenyl (including fused phenyl resulting optionally substituted alkyl such as indenyl, where the phenyl substituent is fused to the parent alkyl moiety), and may have a molecular weight of about 15 to about 100 or about 500.

As used herein the term “aryl” has the broadest meaning generally understood in the art and may include an aromatic ring or aromatic ring system such as phenyl, naphthyl, etc. In some embodiments, aryl may include heteroatoms such as S or N, e.g., thiophenyl, pyridinyl, pyrimidinyl, etc.

The term “heterocyclyl” includes any ring or ring system containing a heteroatom such as N, O, S, P, etc. Heterocyclyl includes heteroaryl rings or ring systems (such as those listed below) and non-aromatic rings or ring systems. Examples of non-aromatic heterocyclyl include azetidinyl, oxatanyl, thietanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxalanyl, dithiolanyl, tetrahydropyranyl, piperidinyl, piperazinyl, morpholino, etc.

The term “heteroaryl” also has the meaning understood by a person of ordinary skill in the art, and includes an “aryl” which has one or more heteroatoms in the ring or ring system, such as pyridinyl, furyl, thienyl, oxazolyl, thiazolyl, imidazolyl, triazolyl, oxadiazolyl, isoxazolyl, indolyl, quinolinyl, benzofuranyl, benzothienyl, benzooxazolyl, benzothiazolyl, benzoimidazolyl, etc.

As used herein, the term “carbocyclyl” has the broadest meaning generally understood in the art and includes rings free of heteroatoms, such as cycloalkyl, e.g. cyclopropyl, cyclobutyl, cyclopentyl, cyclohexyl, etc.; cycloalkenyl, e.g. cyclopropenyl, cyclobutenyl, cyclopentenyl, cyclohexenyl; cycloalkynyl, e.g. cyclopropynyl, cyclobutynyl, cyclopentynyl, cyclohexynyl; bridged cyclocalkyl, e.g. bicyclo[1.1.1]pentane, norborane, etc.; as well as aryl rings free of heteroatoms.

Unless otherwise indicated, any reference to a compound herein by structure, name, or any other means includes pharmaceutically acceptable salts; prodrugs, such as ester prodrugs; alternate solid forms, such as polymorphs, solvates, hydrates, etc.; tautomers; or any other chemical species that may rapidly convert to a compound described herein under conditions in which the compounds are used as described.

A prodrug includes a compound which is converted to a therapeutically active compound after administration, such as by hydrolysis of an ester group or some other biologically labile group.

If stereochemistry is not indicated, a name or structural representation includes any stereoisomer or any mixture of stereoisomers.

Some embodiments include a compound represented by a formula:

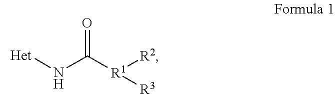

wherein Het is optionally substituted pyrazolo[1,5-a]pyridin-2-yl; R¹ is C₁₋₆ linear or C₁₋₆ branched alkyl; R² is H, OH, CF₃, or C_{3.6}-cycloalkyl-OH; and R³ is H, CF₃, optionally substituted phenyl, optionally substituted pyridinyl, or optionally substituted C₃₋₆ cycloalkyl.

Some embodiments include a compound represented by a formula:

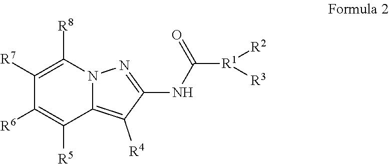

wherein R⁴ is H, F, Cl, Br, I, C₁₋₆ alkyl, C₁₋₆-alkyl-OH, CF₃, CN, C₁₋₆—O-alkyl, optionally substituted C₃₋₆ cycloalkyl, optionally substituted aryl, optionally substituted thiophenyl, or optionally substituted pyridinyl; and R⁵, R⁶, R⁷, and R⁸ are each independently H, F, Cl, Br, I, C₁₋₆ alkyl, C₁₋₆-alkyl-OH, CF₃, CN, C₁₋₆—O-alkyl, optionally substituted C₃₋₆ cycloalkyl, or optionally substituted aryl.

Some embodiments include a composition comprising a compound described herein, such as a compound of Formula 1 or Formula 2, wherein the composition is pharmaceutically acceptable.

Some embodiments include a pharmaceutical dosage form comprising a compound described herein, such as a compound of Formula 1 or Formula 2.

Some embodiments include a method of activating a Kv7 potassium comprising administering an effective amount of a compound described herein, such as a compound of Formula 1 or Formula 2, to a mammal in need thereof.

Some embodiments include a method of treating a disorder associated with a Kv7 potassium channel comprising administering an effective amount of a compound described herein, such as a compound of Formula 1 or Formula 2, to a mammal in need thereof.

Some embodiments include use of a compound of Formula 1 or Formula 2, in the manufacture of a medicament for treating a disorder associated with a Kv7 potassium channel.

With respect to any relevant structural representation, such as Formula 1, Het is an optionally substituted pyrazolopyridinyl, such as optionally substituted pyrazolo[1,5-a]pyridin-2-yl. If Het is substituted, it may have 1, 2, 3, 4, or 5 substituents. Any substituent may be included on the pyrazolopyridinyl. In some embodiments, some or all of the substituents on the pyrazolopyridinyl may have: from 0 to 10 carbon atoms and from 0 to 10 heteroatoms, wherein each heteroatom is independently: O, N, S, F, Cl, Br, or I (provided that there is at least 1 non-hydrogen atom); and/or a molecular weight of 15 Da to 500 Da. For example, the substituents may be C C₁₋₆ alkyl, such as CH₃, C₂H₅, C₃H₇, cyclic C₃H₅, C₄H₉, cyclic C₄H₇, C₅Hn, cyclic C₅H₉, C₆H₁₃, cyclic C₆Hn, etc.; C₁₋₂₀ alkoxyl; C₁₋₂₀ hydroxyalkyl; halo, such as F, Cl, Br, or I; OH; CN; NO₂; C₁₋₆ fluoroalkyl, such as CF₃, CF₂H, C₂F₅, etc.; optionally substituted phenyl, e.g., methylphenyl, chlorophenyl, or fluorophenyl; optionally substituted heteroaryl, e.g., pyridinyl, thiophenyl etc.; a C₁₋₁₀ ester such as —O₂CCH₃, —CO₂CH₃, —O₂CC₂H₅, —CO2C2H5, —O₂C-phenyl, —CO₂-phenyl, etc.; a C₁₋₁₀ ketone such as —COCH₃, —COC₂H₅, —COC₃H₇, —CO-phenyl, etc.; C₁₋₁₂—O-alkyl such as —OCH₃, —OC₂H₅, —OC₃H₇, etc.; or a C₁₋₁₀ amine such as NH₂, NH(CH₃), N(CH₃)₂, N(CH₃)C₂H₅, etc. In some embodiments a substituent of Het is C₁₋₆ alkyl, C₁₋₆—O-alkyl, F, Cl, Br, OH, CN, NO₂, or CF₃. In some embodiments, Het is:

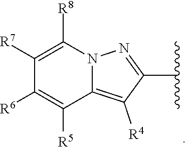

In some embodiments wherein thiophenyl is a substituent of Het, attachment may be at position 2 of the thiophenyl moiety, e.g., thiophen-2-yl, or at any position normally occupied by a hydrogen atom.

With respect to any relevant structural representation, such as Formula 1 and Formula 2, R¹ is C₁₋₆ linear or branched alkyl, such as —CH₂—,

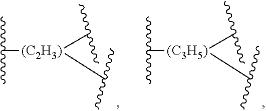

linear or branched —C₄H₈—, linear or branched —C₅H₁₁, etc. In some embodiments, R¹ is —CH₂—,

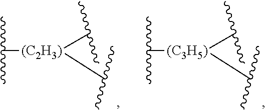

—C₄H₈—, or —C₅H₁₁. In some embodiments, R¹ is —CH₂—. In some embodiments, R¹ is

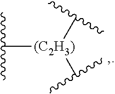

In some embodiments, R¹ is

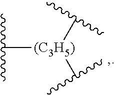

In some embodiments, R¹ is —C₄H₈—. In some embodiments, R¹ is —C₅H₁₁. In some embodiments, R¹ is

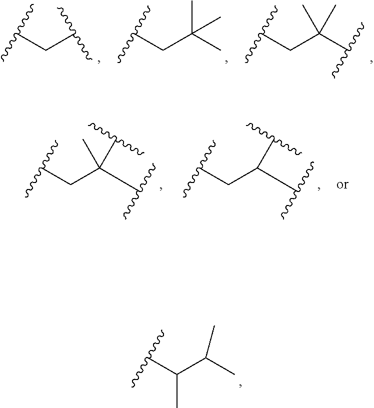

In some embodiments, R¹ is

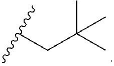 [SMILES: *CC(C)(C)C]

In some embodiments, R¹ is

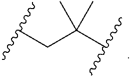 [SMILES: *CC(*)(C)C]

In some embodiments, R¹ is

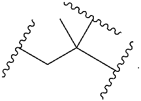 [SMILES: *CC(*)(*)C]

In some embodiments, R¹ is

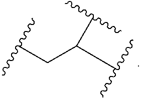 [SMILES: *CC(*)*]

In some embodiments, R¹ is

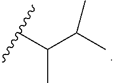 [SMILES: *C(C)C(C)C]

With respect to any relevant structural representation, such as Formula 1 and Formula 2, R² is H, OH, CF₃, or C₃₋₆-cycloalkyl-OH, such as -cyclopropyl-OH, -cyclobutyl-OH, -cyclopentyl-OH, -cyclohexyl-OH. In some embodiments, R² is H, OH, CF₃.

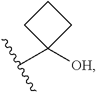 [SMILES: *C1(O)CCC1]

or

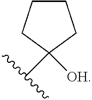 [SMILES: *C1(O)CCCC1]

In some embodiments, R² is H. In some embodiments, R² is CF₃. In some embodiments, R² is

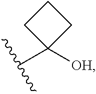 [SMILES: *C1(O)CCC1]

In some embodiments, R² is

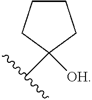 [SMILES: *C1(O)CCCC1]

With respect to any relevant structural representation, such as Formula 1 and Formula 2, R³ is H, CF₃, optionally substituted phenyl, optionally substituted pyridinyl, or optionally substituted C₃₋₆ cycloalkyl, such as optionally substituted cyclopropyl, optionally substituted cyclobutyl, optionally substituted cyclopentyl, optionally substituted cyclohexyl, etc. In some embodiments, R³ is H, CF₃, phenyl, pyridinyl, optionally substituted cyclobutyl, or optionally substituted cyclopentyl. In some embodiments, R³ is H. In some embodiments, R³ is CF₃. In some embodiments, R³ is phenyl. In some embodiments, R³ is pyridinyl. In some embodiments, R³ is optionally substituted cyclobutyl. In some embodiments, R³ is optionally substituted cyclopentyl.

With respect to any relevant structural representation, such as Formula 1 and 2; R⁴, R⁵, R⁶, R⁷, and R⁸ may independently be H or any substituent, such as a substituent having from 0 to 6 carbon atoms and from 0 to 5 heteroatoms, wherein each heteroatom is independently: O, N, S, F, Cl, Br, or I; and/or having a molecular weight of 15 Da to 300 Da, 15 Da to 200 Da, or 15 Da to 150 Da. In some embodiments, R⁴, R⁵, R⁶, R⁷, and R⁸ are independently R^{A}F, Cl, CN, OR^{A}, CF₃, NO₂, NR^{A}R^{B}, COR^{A}, CO₂R^{A}, OCOR^{A}, NR^{A}COR^{B}, CONR^{A}R^{B}, optionally substituted aryl such as optionally substituted phenyl, optionally substituted heteroaryl, such as, thiophenyl, optionally substituted pyridinyl, etc. In some embodiments, R⁵, R⁶, R⁷, and R⁸ are independently H; F; Cl; CN; CF₃; OH; NH₂; C₁₋₆ alkyl, such as methyl, ethyl, propyl isomers (e.g. n-propyl and isopropyl), cyclopropyl, butyl 1 isomers (e.g. cyclobutyl and methylcyclopropyl), pentyl isomers, cyclopentyl isomers, hexyl isomers, cyclohexyl isomers, etc.; or C₁₋₆ alkoxy, such as —O-methyl, —O-ethyl, isomers of —O-propyl, —O-cyclopropyl, isomers of —O-butyl, isomers of —O-cyclobutyl, isomers of —O-pentyl, isomers of —O-cyclopentyl, isomers of —O-hexyl, isomers of —O-cyclohexyl, etc.; optionally substituted phenyl; or optionally substituted pyridinyl.

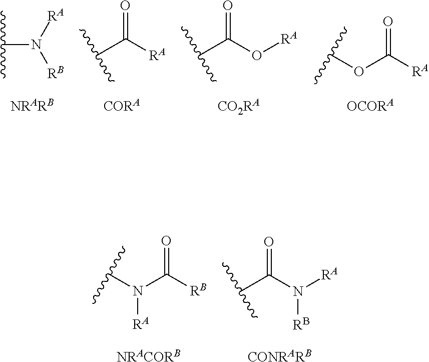

Each R^{A} may independently be H, or C₁₋₁₂ alkyl, including: linear or branched alkyl having a formula CₐHₐ₊₁, or cycloalkyl having a formula CₐHₐ₋₁, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl of a formula: C₃H₅, C₄H₇, C₅H₉, C₆H₁₁, C₇H₁₃, C₈H₁₅, C₉H₁₇, C₁₀H₁₉, etc. In some embodiments, R^{A} may be H or C₁₋₆ alkyl. In some embodiments, R^{A} may be H or C₁₋₃ alkyl. In some embodiments, R^{A} may be H or CH₃. In some embodiments, R^{A} may be H.

Each R^{B} may independently be H, or C₁₋₁₂ alkyl, including: linear or branched alkyl having a formula CₐHₐ₊₁; or cycloalkyl having a formula CₐHₐ, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₈H₁₇, C₇H₁₅, C₉H₁₉, C₁₀H₂₁, etc., or cycloalkyl of a formula: C₃H₅, C₄H₇, C₅H₉, C₆H₁₁, C₇H₁₃, C₈H₁₅, C₉H₁₇, C₁₀H₁₉, etc. In some embodiments, R^{B} may be H or C₁₋₃ alkyl. In some embodiments, R^{B} may be H or CH₃. In some embodiments, R^{B} may be H.

With respect to any relevant structural representation, such as Formula 2, R⁴ is H, or any substituent, such as a substituent having a molecular weight of 15 Da to 50 Da, 75 Da, 100 Da, 150 Da, or 200 Da, and/or consisting of 1, 2, 3, 4, or 5 chemical elements, wherein the chemical elements are C, H, N, O, S, F, Cl, or Br. In some embodiments, R⁴ is H, NO₂, CN, F, Cl, Br, I, CO₂H, OH, NH₂, C₁₋₆ alkylamino, optionally substituted phenyl, optionally substituted pyridinyl, optionally substituted thiophenyl, C₁₋₆ alkyl, or C₁₋₆—O-alkyl. In some embodiments, R⁴ is cyclobutyl, optionally substituted phenyl, 4-fluorophenyl, 4-chlorophenyl, unsubstituted phenyl, 4-methylphenyl, 3-fluorophenyl, 5-chlorothiophen-2-yl, or 6-chloropyridin-3-yl. In some embodiments, R⁴ is H. In some embodiments, R⁴ is cyclobutyl. In some embodiments, R⁴ is optionally substituted pyridinyl, such as an optionally substituted pyridin-3-yl (e.g. 6-chloropyridin-5-yl). In some embodiments, R⁴ is optionally substituted thiophenyl, such as optionally substituted thiopheny-2-yl (e.g. 5-chloro-thiophen-2-yl). In some embodiments, R⁴ is optionally substituted thiophen-2-yl. In some embodiments, R⁴ is optionally substituted phenyl, R⁴ is 4-fluorophenyl, R⁴ is 4-chlorophenyl, R⁴ is unsubstituted phenyl, R⁴ is 4-methylphenyl, R⁴ is 3-fluorophenyl. Additionally, for any embodiments above in this paragraph, R⁵, R⁶, R⁷, and R⁸ can independently be: R^{A} F, Cl, CN, OR^{A} CF₃, NO₂, NR^{A}R^{B}, COR^{A}CO₂R^{A}OCOR^{A} NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁴ is H, cyclobutyl, or optionally substituted phenyl, R⁶, R⁷, and R⁸ can independently be H, C₁₋₄ alkyl, OH, C₁₋₄—O-alkyl, —CHO, C₂₋₄—CO-alkyl, C_{2.4}—CO-alkyl, CO₂H, C_{2.4}—CO₂-alkyl, F, Cl, Br, I, NO₂, CN, optionally substituted phenyl, optionally substituted pyridinyl, or optionally substituted thiophenyl.

With respect to any relevant structural representation, such as Formula 2, R⁵ is H, or any substituent, such as a substituent having a molecular weight of 15 Da to 50 Da, 75 Da, 100 Da, 150 Da, or 200 Da, and/or consisting of 1, 2, 3, 4, or 5 chemical elements, wherein the chemical elements are C, H, N, O, S, F, Cl, or Br. In some embodiments, R⁵ is NO₂, CN, H, F, Cl, Br, I, CO₂H, OH, C₁₋₆ alkylamino, C₁₋₆ alkyl, or C₁₋₆—O-alkyl. In some embodiments, R⁵ is H, Cl, Br, CN, or —OCH₃. In some embodiments, R⁵ is H. Additionally, for any embodiments above in this paragraph, R⁶, R⁷, and R⁸ can independently be: R^{A} F, Cl, CN, OR^{A}, CF₃, NO₂, NR^{A}R^{B}, COR^{A} CO₂R^{A}OCOR^{A}NR^{A}COR^{B}, or CONR^{A}R^{B}; or H, F, Cl, CN, CF₃, OH, NH₂, d.₆ alkyl, or d.₆ alkoxy. In some embodiments wherein R⁵ is H, R⁶, R⁷, and R⁸ can independently be H, C₁₋₄ alkyl, OH, C₁₋₄—O-alkyl, —CHO, C_{2.4}—CO-alkyl, C_{2.4}—CO-alkyl, CO₂H, C_{2.4}—CO₂-alkyl, F, Cl, Br, I, NO₂, CN, optionally substituted phenyl, optionally substituted pyridinyl or optionally substituted thiophenyl.

With respect to any relevant structural representation, such as Formula 2, R⁶ is H, or any substituent, such as a substituent having a molecular weight of 15 Da to 50 Da, 75 Da, 100 Da, 150 Da, or 200 Da, and/or consisting of 1, 2, 3, 4, or 5 chemical elements, wherein the chemical elements are C, H, N, O, S, F, Cl, or Br. In some embodiments, R⁶ is NO₂, CN, H, F, Cl, Br, I, CO₂H, OH, C₁₋₆ alkylamino, C₁₋₆ alkyl, or C₁₋₆—O-alkyl. In some embodiments, R⁶ is H, CF₃, CN, halo (such as F, Cl, or Br), or —OCH₃. In some embodiments, R⁶ is H. In some embodiments, R⁶ is CF₃. In some embodiments, R⁶ is CN. In some embodiments, R⁶ is C₁. In some embodiments, R⁶ is Br. In some embodiments, R⁶ is —OCH₃. Additionally, for any embodiments above in this paragraph, R⁵, R⁷, and R⁸ can independently be: R^{A} F, Cl, CN, OR^{A} CF₃, NO₂, NR^{A}R^{B}, COR^{A} CO₂R^{A}, OCOR^{A} NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁶ is H, CF₃, CN, Cl, Br, or —OCH₃, R⁵, R⁷, and R⁸ can independently be H, C₁₋₄ alkyl, OH, C₁₋₄—O-alkyl, —CHO, C_{2.4}—CO-alkyl, C_{2.4}—CO-alkyl, CO₂H, C_{2.4}—CO₂-alkyl, F, Cl, Br, I, NO₂, CN, optionally substituted phenyl, optionally substituted pyridinyl, or optionally substituted thiophenyl.

With respect to any relevant structural representation, such as Formula 2, R⁷ is H, or any substituent, such as a substituent having a molecular weight of 15 Da to 50 Da, 75 Da, 100 Da, 150 Da, or 200 Da, and/or consisting of 1, 2, 3, 4, or 5 chemical elements, wherein the chemical elements are C, H, N, O, S, F, Cl, or Br. In some embodiments, R⁷ is NO₂, CN, H, F, Cl, Br, I, CO₂H, OH, C₁₋₆ alkylamino, C₁₋₆ alkyl, or C₁₋₆—O-alkyl. In some embodiments, R⁷ is H, C₁, Br, CN, or —OCH₃. In some embodiments, R⁷ is H. Additionally, for any embodiments above in this paragraph, R⁵, R⁶, and R⁸ can independently be: R^{A} F, Cl, CN, OR^{A}, CF₃, NO₂, NR^{A}R^{B}, COR^{A} CO₂R^{A}OCOR^{A} NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁷ is H, R⁵, R⁶ and R⁸ can independently be H, C₁₋₄ alkyl, OH, C₁₋₄—O-alkyl, —CHO, C_{2.4}—CO-alkyl, C_{2.4}—CO-alkyl, CO₂H, C_{2.4}—CO₂-alkyl, F, Cl, Br, I, NO₂, CN, optionally substituted phenyl, optionally substituted pyridinyl, or optionally substituted thiophenyl.

With respect to any relevant structural representation, such as Formula 2, R⁸ is H, or any substituent, such as a substituent having a molecular weight of 15 Da to 50 Da, 75 Da, 100 Da, 150 Da, or 200 Da, and/or consisting of 1, 2, 3, 4, or 5 chemical elements, wherein the chemical elements are C, H, N, O, S, F, Cl, or Br. In some embodiments, R⁸ is NO₂, CN, H, F, Cl, Br, I, CO₂H, OH, C₁₋₆ alkylamino, C₁₋₆ alkyl, or C₁₋₆—O-alkyl. In some embodiments, R⁸ is H, C₁, Br, CN, or —OCH₃. In some embodiments, R⁸ is H or Br. In some embodiments, R⁸ is H. In some embodiments, R⁸ is Br. Additionally, for any embodiments above in this paragraph, R⁵, R⁶, and R⁷ can independently be: R^{A} F, Cl, CN, OR^{A}, CF₃, NO₂, NR^{A}R^{B}, COR^{A}CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁸ is H or Br; R⁵, R⁶, and R⁷ can independently be H, C1-4 alkyl, OH, C₁₋₄—O-alkyl, —CHO, C_{2.4}—CO-alkyl, C_{2.4}—CO-alkyl, CO₂H, C_{2.4}—CO₂-alkyl, F, Cl, Br, I, NO₂, CN, optionally substituted phenyl, optionally substituted pyridinyl, or optionally substituted thiophenyl.

Table 1 shows the structures of the various Example embodiments prepared by the methods disclosed herein and indicates the general coupling method used, together with a summary of the LCMS analytical data.

TABLE 1
List of Examples, Synthetic Methods, and Analytical Data

| | | | | | HPLC | |
|---|---|---|---|---|---|---|
| | | | | | Retention | |
| | | | Coupling | HPLC | time | LC/ |
| Example | Structure | Name | Method | Method | (min) | MS m/z |
| 1 | 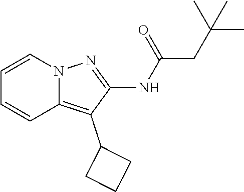 [SMILES: CC(C)(C)CC(=O)Nc1nn2ccccc2c1C1CCC1] | N-(3- cyclobutylpyrazolo[1, 5-a]pyridin-2-yl)-3,3- dimethylbutanamide | 8 | B | 3.59 | 285.4 |
| 2 | 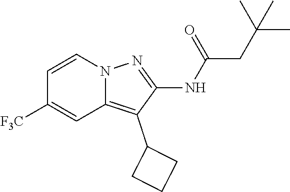 [SMILES: CC(C)(C)CC(=O)Nc1nn2ccc(C(F)(F)F)cc2c1C1CCC1] | N-(3-cyclobutyl-5- (trifluoromethyl)pyrazolo [1,5-a]pyridin-2- yl)-3,3- dimethylbutanamide | 8 | B | 4.11 | 353.4 |
| 3 | 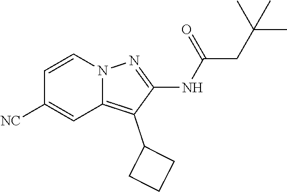 [SMILES: CC(C)(C)CC(=O)Nc1nn2ccc(C#N)cc2c1C1CCC1] | N-(5-cyano-3- cyclobutylpyrazolo[1, 5-a]pyridin-2-yl)-3,3- dimethylbutanamide | 8 | B | 3.63 | 310.4 |
| 4 | 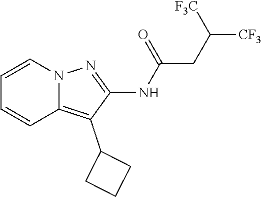 [SMILES: O=C(CC(C(F)(F)F)C(F)(F)F)Nc1nn2ccccc2c1C1CCC1] | N-(3- cyclobutylpyrazolo[1, 5-a]pyridin-2-yl)- 4,4,4-trifluoro-3- (trifluoromethyl) butanamide | 5 | B | 3.86 | 379.3 |
| 5 | 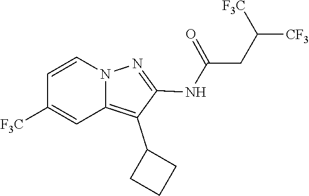 [SMILES: O=C(CC(C(F)(F)F)C(F)(F)F)Nc1nn2ccc(C(F)(F)F)cc2c1C1CCC1] | N-(3-cyclobutyl-5- (trifluoromethyl)pyrazolo [1,5-a]pyridin-2- yl)-4,4,4-trifluoro-3- (trifluoromethyl) butanamide | 5 | B | 4.22 | 447.3 |
| 6 | 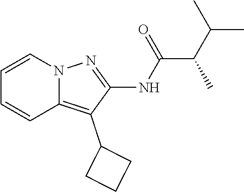 [SMILES: CC(C)[C@H](C)C(=O)Nc1nn2ccccc2c1C1CCC1] | (S)-N-(3- cyclobutylpyrazolo[1, 5-a]pyridin-2-yl)-2,3- dimethylbutanamide | 5 | B | 3.54 | 285.4 |
| 7 | 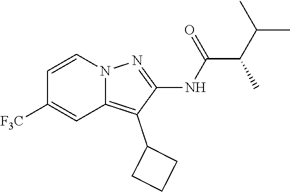 [SMILES: CC(C)[C@H](C)C(=O)Nc1nn2ccc(C(F)(F)F)cc2c1C1CCC1] | (S)-N-(3-cyclobutyl-5- (trifluoromethyl)pyrazolo [1,5-a]pyridin-2-yl)-2,3- dimethylbutanamide | 5 | B | 4.06 | 353.4 |
| 8 | 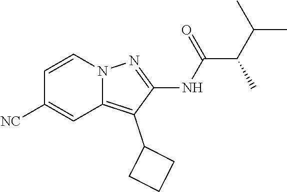 [SMILES: CC(C)[C@H](C)C(=O)Nc1nn2ccc(C#N)cc2c1C1CCC1] | (S)-N-(5-cyano-3- cyclobutylpyrazolo[1, 5-a]pyridin-2-yl)-2,3- dimethylbutanamide | 5 | B | 3.58 | 310.4 |
| 9 | 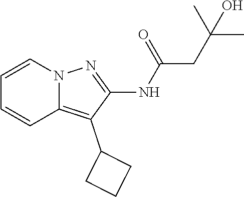 [SMILES: CC(C)(O)CC(=O)Nc1nn2ccccc2c1C1CCC1] | N-(3- cyclobutylpyrazolo[1, 5-a]pyridin-2-yl)-3- hydroxy-3- methylbutanamide | 5 | B | 2.84 | 287.4 |
| 10 | 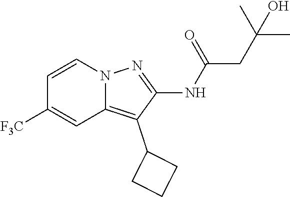 [SMILES: CC(C)(O)CC(=O)Nc1nn2ccc(C(F)(F)F)cc2c1C1CCC1] | N-(3-cyclobutyl-5- (trifluoromethyl)pyrazolo [1,5-a]pyridin-2- yl)-3-hydroxy-3- methylbutanamide | 5 | B | 3.40 | 355.4 |
| 11 | 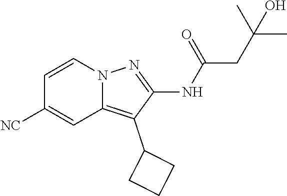 [SMILES: CC(C)(O)CC(=O)Nc1nn2ccc(C#N)cc2c1C1CCC1] | N-(5-cyano-3- cyclobutylpyrazolo[1, 5-a]pyridin-2-yl)-3- hydroxy-3- methylbutanamide | 5 | B | 2.86 | 313.2 |
| 12 | 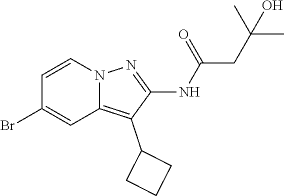 [SMILES: CC(C)(O)CC(=O)Nc1nn2ccc(Br)cc2c1C1CCC1] | N-(5-bromo-3- cyclobutylpyrazolo[1, 5-a]pyridin-2-yl)-3- hydroxy-3- methylbutanamide | 5 | B | 3.26 | 366.0 |
| 13 | 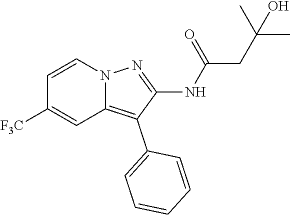 [SMILES: CC(C)(O)CC(=O)Nc1nn2ccc(C(F)(F)F)cc2c1-c1ccccc1] | 3-hydroxy-3-methyl- N-(3-phenyl-5- (trifluoromethyl)pyrazolo [1,5-a]pyridin-2- yl)butanamide | 5 | B | 3.37 | 378.0 |
| 14 | 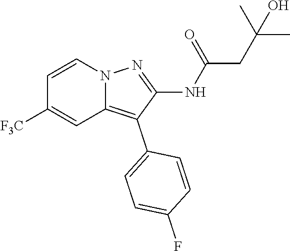 [SMILES: CC(C)(O)CC(=O)Nc1nn2ccc(C(F)(F)F)cc2c1-c1ccc(F)cc1] | N-(3-(4- fluorophenyl)-5- (trifluoromethyl)pyrazolo [1,5-a]pyridin-2- yl)-3-hydroxy-3- methylbutanamide | 5 | B | 3.41 | 395.4 |
| 16 | 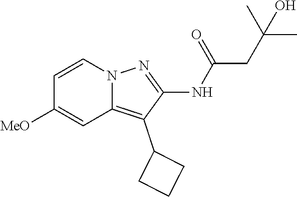 [SMILES: COc1ccn2nc(NC(=O)CC(C)(C)O)c(C3CCC3)c2c1] | N-(3-cyclobutyl-5- methoxypyrazolo[1,5-a] pyridin-2-yl)-3- hydroxy-3- methylbutanamide | 6 | B | 2.87 | 318.4 |
| 17 | 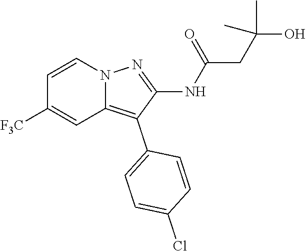 [SMILES: CC(C)(O)CC(=O)Nc1nn2ccc(C(F)(F)F)cc2c1-c1ccc(Cl)cc1] | N-(3-(4- chlorophenyl)-5- (trifluoromethyl)pyrazolo [1,5-a]pyridin-2- yl)-3-hydroxy-3- methylbutanamide | 5 | B | 3.62 | 412.0 |
| 18 | 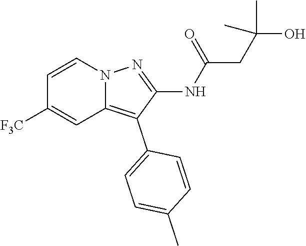 [SMILES: Cc1ccc(-c2c(NC(=O)CC(C)(C)O)nn3ccc(C(F)(F)F)cc23)cc1] | 3-hydroxy-3-methyl- N-(3-(p-tolyl)-5- (trifluoromethyl)pyrazolo [1,5-a]pyridin-2- yl)butanamide | 5 | B | 3.56 | 391.4 |
| 19 | 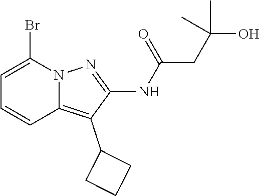 [SMILES: CC(C)(O)CC(=O)Nc1nn2c(Br)cccc2c1C1CCC1] | N-(7-bromo-3- cyclobutylpyrazolo[1, 5-a]pyridin-2-yl)-3- hydroxy-3- methylbutanamide | 6 | B | 3.18 | 366.0/ 368.0 |
| 20 | 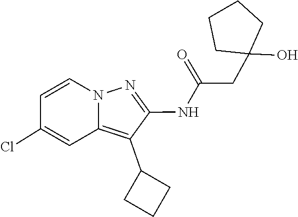 [SMILES: O=C(CC1(O)CCCC1)Nc1nn2ccc(Cl)cc2c1C1CCC1] | N-(5-chloro-3- cyclobutylpyrazolo[1, 5-a]pyridin-2-yl)-2- (1- hydroxycyclopentyl) acetamide | 6 | B | 3.60 | 348.4 |
| 21 | 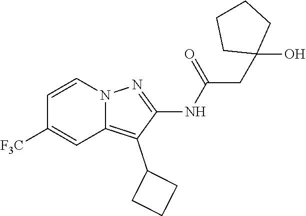 [SMILES: O=C(CC1(O)CCCC1)Nc1nn2ccc(C(F)(F)F)cc2c1C1CCC1] | N-(3-cyclobutyl-5- (trifluoromethyl)pyrazolo [1,5-a]pyridin-2- yl)-2-(1- hydroxycyclopentyl) acetamide | 6 | B | 3.73 | 382.4 |
| 22 | 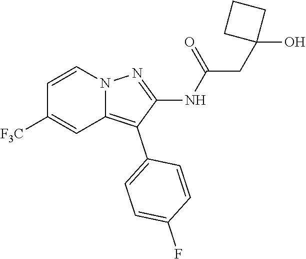 [SMILES: O=C(CC1(O)CCC1)Nc1nn2ccc(C(F)(F)F)cc2c1-c1ccc(F)cc1] | N-(3-(4- fluorophenyl)-5- (trifluoromethyl)pyrazolo [1,5-a]pyridin-2- yl)-2-(1- hydroxycyclobutyl) acetamide | 5 | B | 3.52 | 408.0 |
| 23 | 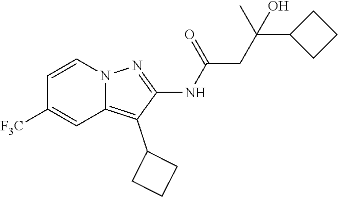 [SMILES: CC(O)(CC(=O)Nc1nn2ccc(C(F)(F)F)cc2c1C1CCC1)C1CCC1] | 3-cyclobutyl-N-(3- cyclobutyl-5- (trifluoromethyl)pyrazolo [1,5-a]pyridin-2- yl)-3- hydroxybutanamide | 5 | B | 3.93 | 396.4 |
| 24 | 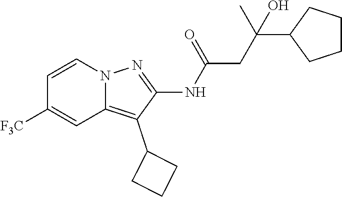 [SMILES: CC(O)(CC(=O)Nc1nn2ccc(C(F)(F)F)cc2c1C1CCC1)C1CCCC1] | N-(3-cyclobutyl-5- (trifluoromethyl)pyrazolo [1,5-a]pyridin-2- yl)-3-cyclopentyl-3- hydroxybutanamide | 6 | B | 3.98 | 431.5 |
| 25 | 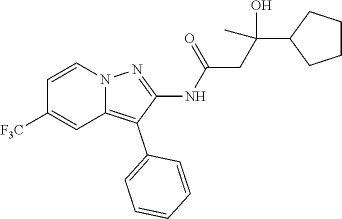 [SMILES: CC(O)(CC(=O)Nc1nn2ccc(C(F)(F)F)cc2c1-c1ccccc1)C1CCCC1] | 3-cyclopentyl-3- hydroxy-N-(3- phenyl-5- (trifluoromethyl)pyrazolo [1,5-a]pyridin-2- yl)butanamide | 6 | B | 3.98 | 410.4 |
| 26 | 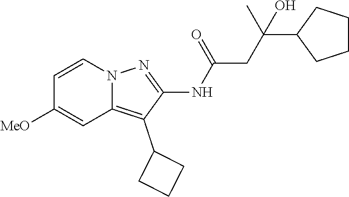 [SMILES: COc1ccn2nc(NC(=O)CC(C)(O)C3CCCC3)c(C3CCC3)c2c1] | N-(3-cyclobutyl-5- methoxypyrazolo[1,5-a] pyridin-2-yl)-3- cyclopentyl-3- hydroxybutanamide | 6 | B | 3.61 | 372.4 |
| 27 | 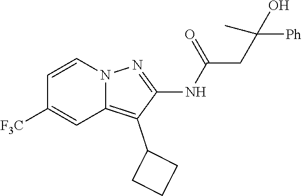 [SMILES: CC(O)(CC(=O)Nc1nn2ccc(C(F)(F)F)cc2c1C1CCC1)c1ccccc1] | N-(3-cyclobutyl-5- (trifluoromethyl)pyrazolo [1,5-a]pyridin-2- yl)-3-hydroxy-3- phenylbutanamide | 5 | B | 3.93 | 417.4 |
| 28 | 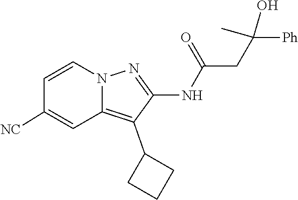 [SMILES: CC(O)(CC(=O)Nc1nn2ccc(C#N)cc2c1C1CCC1)c1ccccc1] | N-(5-cyano-3- cyclobutylpyrazolo[1, 5-a]pyridin-2-yl)-3- hydroxy-3- phenylbutanamide | 5 | B | 3.46 | 374.4 |
| 29 | 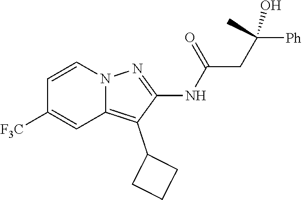 [SMILES: C[C@](O)(CC(=O)Nc1nn2ccc(C(F)(F)F)cc2c1C1CCC1)c1ccccc1] | (S)-N-(3-cyclobutyl- 5- (trifluoromethyl)pyrazolo [1,5-a]pyridin-2- yl)-3-hydroxy-3- phenylbutanamide | 6 | B | 3.93 | 417.4 |
| 30 | 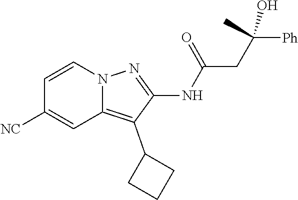 [SMILES: C[C@](O)(CC(=O)Nc1nn2ccc(C#N)cc2c1C1CCC1)c1ccccc1] | (S)-N-(5-cyano-3- cyclobutylpyrazolo[1, 5-a]pyridin-2-yl)-3- hydroxy-3- phenylbutanamide | 6 | B | 3.44 | 374.4 |
| 31 | 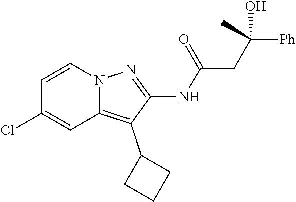 [SMILES: C[C@](O)(CC(=O)Nc1nn2ccc(Cl)cc2c1C1CCC1)c1ccccc1] | (S)-N-(5-chloro-3- cyclobutylpyrazolo[1, 5-a]pyridin-2-yl)-3- hydroxy-3- phenylbutanamide | 6 | B | 3.80 | 384.4 |
| 32 | 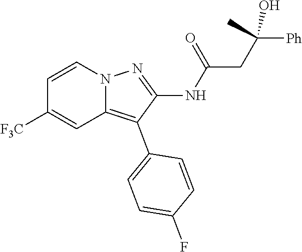 [SMILES: C[C@](O)(CC(=O)Nc1nn2ccc(C(F)(F)F)cc2c1-c1ccc(F)cc1)c1ccccc1] | (S)-N-(3-(4- fluorophenyl)-5- (trifluoromethyl)pyrazolo [1,5-a]pyridin-2- yl)-3-hydroxy-3- phenylbutanamide | 6 | B | 3.85 | 457.4 |
| 33 | 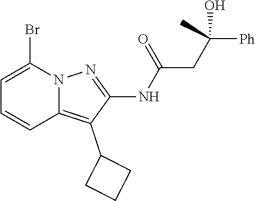 [SMILES: C[C@](O)(CC(=O)Nc1nn2c(Br)cccc2c1C1CCC1)c1ccccc1] | (S)-N-(7-bromo-3- cyclobutylpyrazolo[1, 5-a]pyridin-2-yl)-3- hydroxy-3- phenylbutanamide | 6 | B | 3.80 | 428.0/ 430.0 |
| 34 | 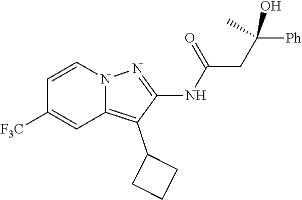 [SMILES: C[C@@](O)(CC(=O)Nc1nn2ccc(C(F)(F)F)cc2c1C1CCC1)c1ccccc1] | (R)-N-(3-cyclobutyl- 5- (trifluoromethyl)pyrazolo [1,5-a]pyridin-2- yl)-3-hydroxy-3- phenylbutanamide | 6 | B | 3.91 | 417.4 |
| 35 | 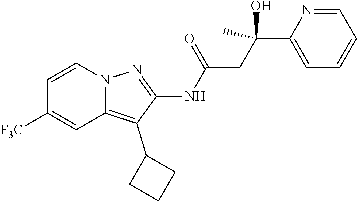 [SMILES: C[C@@](O)(CC(=O)Nc1nn2ccc(C(F)(F)F)cc2c1C1CCC1)c1ccccn1] | (R)-N-(3-cyclobutyl- 5- (trifluoromethyl)pyrazolo [1,5-a]pyridin-2- yl)-3-hydroxy-3- (pyridin-2- yl)butanamide | 7 | B | 2.88 | 418.4 |
| 36 | 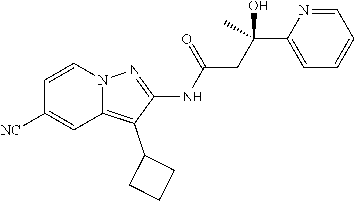 [SMILES: C[C@@](O)(CC(=O)Nc1nn2ccc(C#N)cc2c1C1CCC1)c1ccccn1] | (R)-N-(5-cyano-3- cyclobutylpyrazolo[1, 5-a]pyridin-2-yl)-3- hydroxy-3-(pyridin- 2-yl)butanamide | 7 | B | 2.38 | 375.4 |
| 37 | 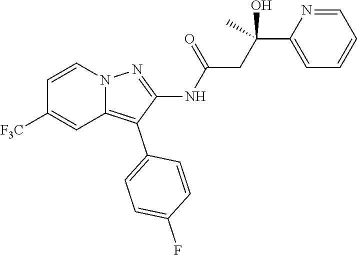 [SMILES: C[C@@](O)(CC(=O)Nc1nn2ccc(C(F)(F)F)cc2c1-c1ccc(F)cc1)c1ccccn1] | (R)-N-(3-(4- fluorophenyl)-5- (trifluoromethyl)pyrazolo [1,5-a]pyridin-2- yl)-3-hydroxy-3- (pyridin-2- yl)butanamide | 7 | B | 2.97 | 458.4 |
| 38 | 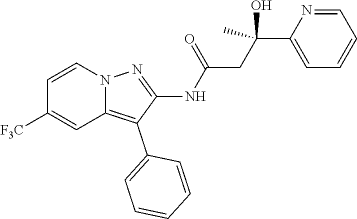 [SMILES: C[C@@](O)(CC(=O)Nc1nn2ccc(C(F)(F)F)cc2c1-c1ccccc1)c1ccccn1] | (R)-3-hydroxy-N-(3- phenyl-5- (trifluoromethyl)pyrazolo [1,5-a]pyridin-2- yl)-3-(pyridin-2- yl)butanamide | 7 | B | 2.87 | 440.4 |
| 39 | 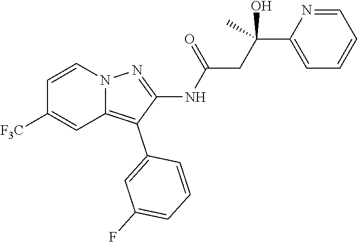 [SMILES: C[C@@](O)(CC(=O)Nc1nn2ccc(C(F)(F)F)cc2c1-c1cccc(F)c1)c1ccccn1] | (R)-N-(3-(3- fluorophenyl)-5- (trifluoromethyl)pyrazolo [1,5-a]pyridin-2- yl)-3-hydroxy-3- (pyridin-2- yl)butanamide | 7 | B | 2.94 | 458.4 |
| 40 | 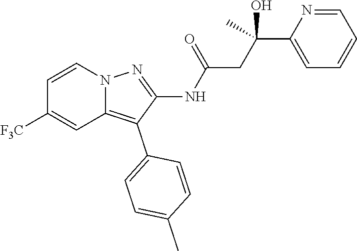 [SMILES: Cc1ccc(-c2c(NC(=O)C[C@@](C)(O)c3ccccn3)nn3ccc(C(F)(F)F)cc23)cc1] | (R)-3-hydroxy-3- (pyridin-2-yl)-N-(3- (p-tolyl)-5- (trifluoromethyl) pyrazolo[1,5-a]pyridin-2- yl)butanamide | 7 | B | 3.11 | 454.4 |
| 41 | 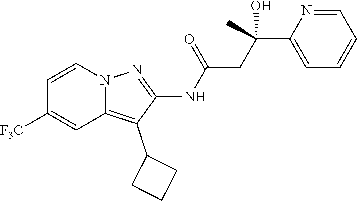 [SMILES: C[C@](O)(CC(=O)Nc1nn2ccc(C(F)(F)F)cc2c1C1CCC1)c1ccccn1] | (S)-N-(3-cyclobutyl-5- (trifluoromethyl)pyrazolo [1,5-a]pyridin-2- yl)-3-hydroxy-3- (pyridin-2- yl)butanamide | 7 | B | 2.89 | 418.4 |
| 42 | 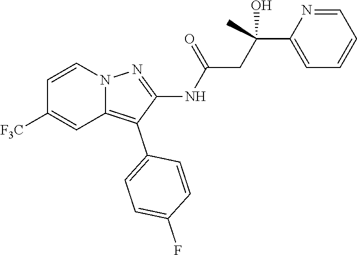 [SMILES: C[C@](O)(CC(=O)Nc1nn2ccc(C(F)(F)F)cc2c1-c1ccc(F)cc1)c1ccccn1] | (S)-N-(3-(4- fluorophenyl)-5- (trifluoromethyl)pyrazolo [1,5-a]pyridin-2- yl)-3-hydroxy-3- (pyridin-2- yl)butanamide | 7 | A | 1.70 | 458.4 |
| 43 | 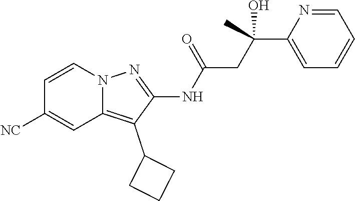 [SMILES: C[C@](O)(CC(=O)Nc1nn2ccc(C#N)cc2c1C1CCC1)c1ccccn1] | (S)-N-(5-cyano-3- cyclobutylpyrazolo[1, 5-a]pyridin-2-yl)-3- hydroxy-3-(pyridin- 2-yl)butanamide | 7 | A | 1.44 | 375.4 |
| 44 | 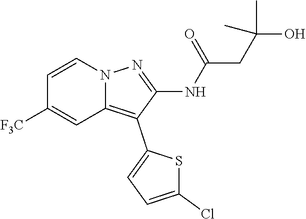 [SMILES: CC(C)(O)CC(=O)Nc1nn2ccc(C(F)(F)F)cc2c1-c1ccc(Cl)s1] | N-(3-(5- chlorothiophen-2-yl)- 5- (trifluoromethyl)pyrazolo [1,5-a]pyridin-2- yl)-3-hydroxy-3- methylbutanamide | 5 | B | 3.68 | 417.8 |
| 45 | 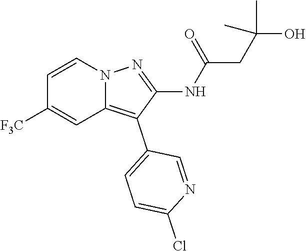 [SMILES: CC(C)(O)CC(=O)Nc1nn2ccc(C(F)(F)F)cc2c1-c1ccc(Cl)nc1] | N-(3-(6- chloropyridin-3-yl)-5- (trifluoromethyl)pyrazolo [1,5-a]pyridin-2- yl)-3-hydroxy-3- methylbutanamide | 6 | B | 3.16 | 412.8 |
| 46 | 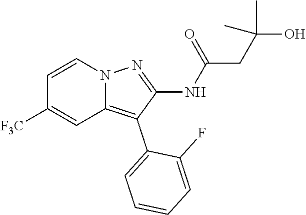 [SMILES: CC(C)(O)CC(=O)Nc1nn2ccc(C(F)(F)F)cc2c1-c1ccccc1F] | N-(3-(2- fluorophenyl)-5- (trifluoromethyl)pyrazolo [1,5-a]pyridin-2- yl)-3-hydroxy-3- methylbutanamide | | | 3.36 | 396.4 |
| 47 | 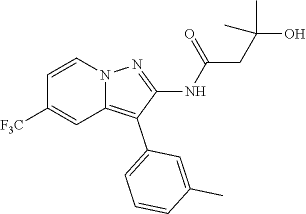 [SMILES: Cc1cccc(-c2c(NC(=O)CC(C)(C)O)nn3ccc(C(F)(F)F)cc23)c1] | 3-hydroxy-3-methyl- N-(3-(m-tolyl)-5- (trifluoromethyl)pyrazolo [1,5-a]pyridin-2- yl)butanamide | | | 3.54 | 392.4 |
| 48 | 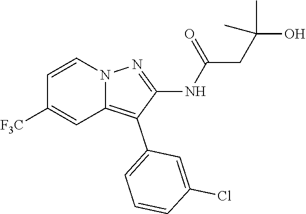 [SMILES: CC(C)(O)CC(=O)Nc1nn2ccc(C(F)(F)F)cc2c1-c1cccc(Cl)c1] | N-(3-(3- chlorophenyl)-5- (trifluoromethyl)pyrazolo [1,5-a]pyridin-2- yl)-3-hydroxy-3- methylbutanamide | | | 3.61 | 412.0 |
| 49 | 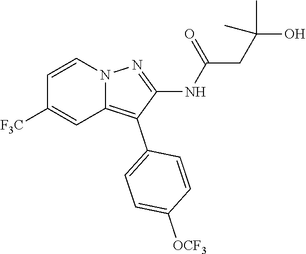 [SMILES: CC(C)(O)CC(=O)Nc1nn2ccc(C(F)(F)F)cc2c1-c1ccc(OC(F)(F)F)cc1] | 3-hydroxy-3-methyl- N-(3-(4- (trifluoromethoxy) phenyl)-5- (trifluoromethyl)pyrazolo [1,5-a]pyridin-2- yl)butanamide | | | 3.79 | 462.0 |
| 50 | 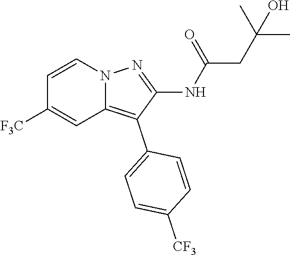 [SMILES: CC(C)(O)CC(=O)Nc1nn2ccc(C(F)(F)F)cc2c1-c1ccc(C(F)(F)F)cc1] | 3-hydroxy-3-methyl- N-(5- (trifluoromethyl)-3-(4- (trifluoromethyl) phenyl)pyrazolo [1,5- a]pyridin-2- yl)butanamide | | | 3.72 | 446.4 |
| 51 | 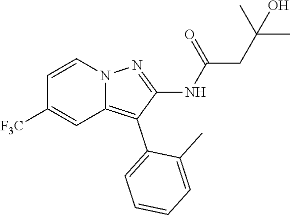 [SMILES: Cc1ccccc1-c1c(NC(=O)CC(C)(C)O)nn2ccc(C(F)(F)F)cc12] | 3-hydroxy-3-methyl- N-(3-(o-tolyl)-5- (trifluoromethyl)pyrazolo [1,5-a]pyridin-2- yl)butanamide | | | 3.50 | 392.4 |
| 52 | 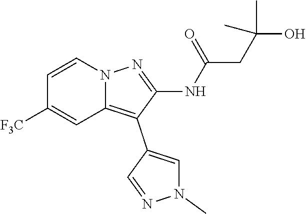 [SMILES: Cn1cc(-c2c(NC(=O)CC(C)(C)O)nn3ccc(C(F)(F)F)cc23)cn1] | 3-hydroxy-3-methyl- N-(3-(1-methyl-1H- pyrazol-4-yl)-5- (trifluoromethyl)pyrazolo [1,5-a]pyridin-2- yl)butanamide | | | 2.71 | 382.4 |
| 53 | 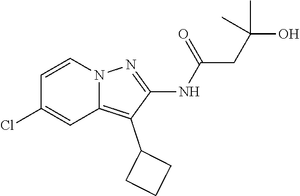 [SMILES: CC(C)(O)CC(=O)Nc1nn2ccc(Cl)cc2c1C1CCC1] | N-(5-chloro-3- cyclobutylpyrazolo[1, 5-a]pyridin-2-yl)-3- hydroxy-3- methylbutanamide | | | 3.21 | 322.0 |
| 54 | 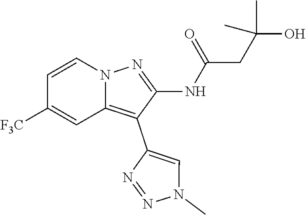 [SMILES: Cn1cc(-c2c(NC(=O)CC(C)(C)O)nn3ccc(C(F)(F)F)cc23)nn1] | 3-hydroxy-3-methyl- N-(3-(1-methyl-1H- 1,2,3-triazol-4-yl)-5- (trifluoromethyl)pyrazolo [1,5-a]pyridin-2- yl)butanamide | | | 2.74 | 383.2 |
| 55 | 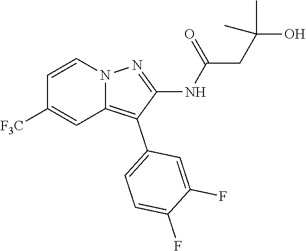 [SMILES: CC(C)(O)CC(=O)Nc1nn2ccc(C(F)(F)F)cc2c1-c1ccc(F)c(F)c1] | N-(3-(3,4- difluorophenyl)-5- (trifluoromethyl)pyrazolo [1,5-a]pyridin-2- yl)-3-hydroxy-3- methylbutanamide | | | 3.52 | 414.0 |
| 56 | 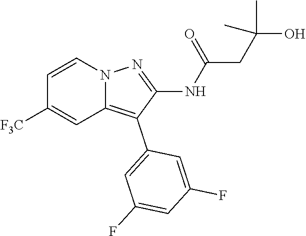 [SMILES: CC(C)(O)CC(=O)Nc1nn2ccc(C(F)(F)F)cc2c1-c1cc(F)cc(F)c1] | N-(3-(3,5- difluorophenyl)-5- (trifluoromethyl)pyrazolo [1,5-a]pyridin-2- yl)-3-hydroxy-3- methylbutanamide | | | 3.51 | 414.0 |
| 57 | 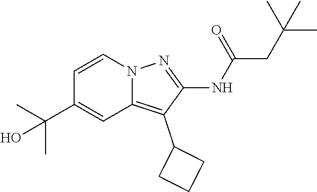 [SMILES: CC(C)(C)CC(=O)Nc1nn2ccc(C(C)(C)O)cc2c1C1CCC1] | N-(3-cyclobutyl-5-(2- hydroxypropan-2- yl)pyrazolo[1,5- a]pyridin-2-yl)-3,3- dimethylbutanamide | | | 3.25 | 344.4 |
| 58 | 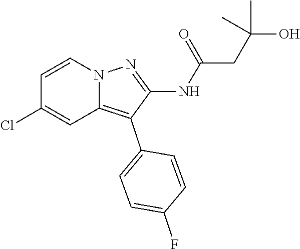 [SMILES: CC(C)(O)CC(=O)Nc1nn2ccc(Cl)cc2c1-c1ccc(F)cc1] | N-(5-chloro-3-(4- fluorophenyl)pyrazolo [1,5-a]pyridin-2-yl)- 3-hydroxy-3- methylbutanamide | | | 3.27 | 362.0 |
| 59 | 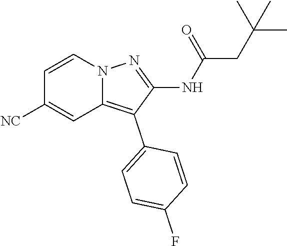 [SMILES: CC(C)(C)CC(=O)Nc1nn2ccc(C#N)cc2c1-c1ccc(F)cc1] | N-(5-cyano-3-(4- fluorophenyl)pyrazolo [1,5-a]pyridin-2-yl)- 3,3-dimethylbutanamide | | | 3.62 | 351.2 |
| 60 | 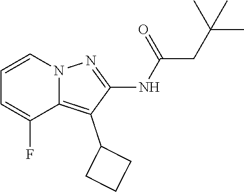 [SMILES: CC(C)(C)CC(=O)Nc1nn2cccc(F)c2c1C1CCC1] | N-(3-cyclobutyl-4- fluoropyrazolo [1,5- a]pyridin-2-yl)-3,3- dimethylbutanamide | | | 3.66 | 304.4 |
| 61 | 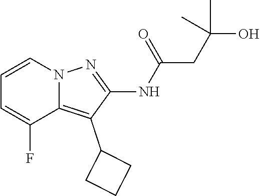 [SMILES: CC(C)(O)CC(=O)Nc1nn2cccc(F)c2c1C1CCC1] | N-(3-cyclobutyl-4- fluoropyrazolo[1,5- a]pyridin-2-yl)-3- hydroxy-3- methylbutanamide | | | 3.02 | 306.4 |
| 62 | 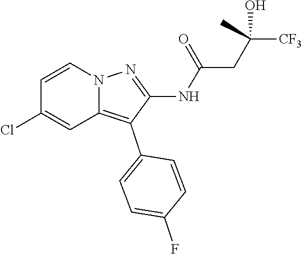 [SMILES: C[C@](O)(CC(=O)Nc1nn2ccc(Cl)cc2c1-c1ccc(F)cc1)C(F)(F)F] | (S)-N-(5-chloro-3-(4- fluorophenyl)pyrazolo [1,5-a]pyridin-2-yl)- 4,4,4-trifluoro-3- hydroxy-3- methylbutanamide | | | 3.63 | 416.0 |
| 63 | 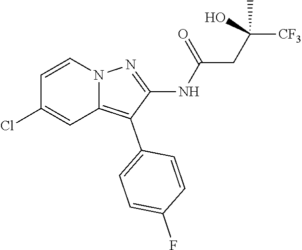 [SMILES: C[C@@](O)(CC(=O)Nc1nn2ccc(Cl)cc2c1-c1ccc(F)cc1)C(F)(F)F] | (R)-N-(5-chloro-3-(4- fluorophenyl)pyrazolo [1,5-a]pyridin-2-yl)- 4,4,4-trifluoro-3- hydroxy-3- methylbutanamide | | | 3.63 | 416.0 |
| 64 | 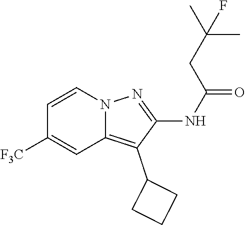 [SMILES: CC(C)(F)CC(=O)Nc1nn2ccc(C(F)(F)F)cc2c1C1CCC1] | N-(3-cyclobutyl-5- (trifluoromethyl)pyrazolo [1,5-a]pyridin-2- yl)-3-fluoro-3- methylbutanamide | | | 3.77 | 358.4 |
| 65 | 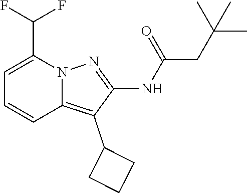 [SMILES: CC(C)(C)CC(=O)Nc1nn2c(C(F)F)cccc2c1C1CCC1] | N-(3-cyclobutyl-7- (difluoromethyl)pyrazolo [1,5-a]pyridin-2- yl)-3,3- dimethylbutanamide | | | 3.95 | 336.4 |
| 66 | 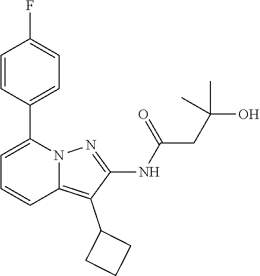 [SMILES: CC(C)(O)CC(=O)Nc1nn2c(-c3ccc(F)cc3)cccc2c1C1CCC1] | N-(3-cyclobutyl-7-(4- fluorophenyl)pyrazolo [1,5-a]pyridin-2-yl)- 3-hydroxy-3- methylbutanamide | | | 3.73 | 382.4 |
| 67 | 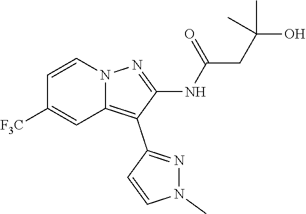 [SMILES: Cn1ccc(-c2c(NC(=O)CC(C)(C)O)nn3ccc(C(F)(F)F)cc23)n1] | 3-hydroxy-3-methyl- N-(3-(1-methyl-1H- pyrazol-3-yl)-5- (trifluoromethyl)pyrazolo [1,5-a]pyridin-2- yl)butanamide | | | 2.97 | 382.4 |
| 68 | 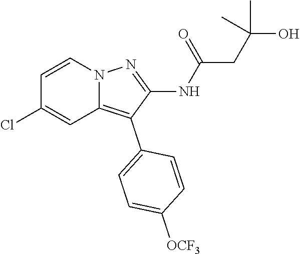 [SMILES: CC(C)(O)CC(=O)Nc1nn2ccc(Cl)cc2c1-c1ccc(OC(F)(F)F)cc1] | N-(5-chloro-3-(4- (trifluoromethoxy) phenyl)pyrazolo[1,5- a]pyridin-2-yl)-3- hydroxy-3- methylbutanamide | | | 3.60 | 428.0 |
| 69 | 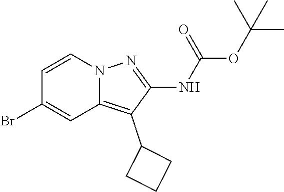 [SMILES: CC(C)(C)OC(=O)Nc1nn2ccc(Br)cc2c1C1CCC1] | tert-butyl (5-bromo- 3- cyclobutylpyrazolo[1, 5-a]pyridin-2- yl)carbamate | | | 4.19 | no ionization observed in either pos. or neg. modes |
| 70 | 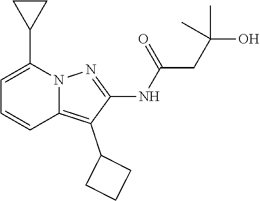 [SMILES: CC(C)(O)CC(=O)Nc1nn2c(C3CC3)cccc2c1C1CCC1] | N-(3-cyclobutyl-7- cyclopropylpyrazolo [1,5-a]pyridin-2-yl)-3- hydroxy-3- methylbutanamide | | | 3.26 | 328.4 |
| 71 | 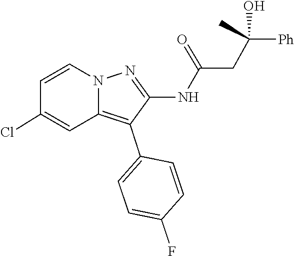 [SMILES: C[C@](O)(CC(=O)Nc1nn2ccc(Cl)cc2c1-c1ccc(F)cc1)c1ccccc1] | (S)-N-(5-chloro-3-(4- fluorophenyl)pyrazolo [1,5-a]pyridin-2-yl)- 3-hydroxy-3- phenylbutanamide | | | 3.70 | 424.0 |
| 72 | 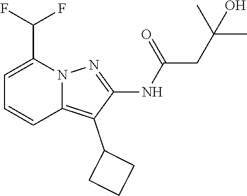 [SMILES: CC(C)(O)CC(=O)Nc1nn2c(C(F)F)cccc2c1C1CCC1] | N-(3-cyclobutyl-7- (difluoromethyl)pyrazolo [1,5-a]pyridin-2- yl)-3-hydroxy-3- methylbutanamide | | | 3.21 | 338.4 |
| 73 | 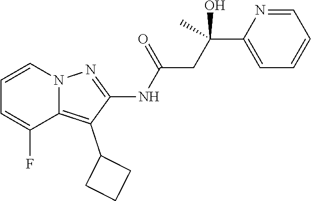 [SMILES: C[C@@](O)(CC(=O)Nc1nn2cccc(F)c2c1C1CCC1)c1ccccn1] | (R)-N-(3-cyclobutyl- 4-fluoropyrazolo [1,5- a]pyridin-2-yl)-3- hydroxy-3-(pyridin- 2-yl)butanamide | | | 1.53 | 369.2 |
| 74 | 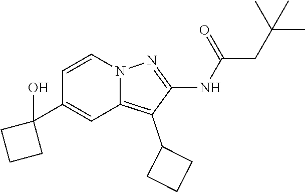 [SMILES: CC(C)(C)CC(=O)Nc1nn2ccc(C3(O)CCC3)cc2c1C1CCC1] | N-(3-cyclobutyl-5-(1- hydroxycyclobutyl) pyrazolo[1,5- a]pyridin-2-yl)-3,3- dimethylbutanamide | | | 3.44 | 356.4 |
| 75 | 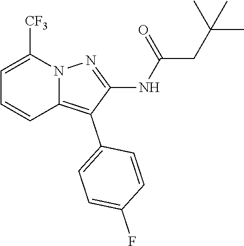 [SMILES: CC(C)(C)CC(=O)Nc1nn2c(C(F)(F)F)cccc2c1-c1ccc(F)cc1] | N-(3-(4- fluorophenyl)-7- (trifluoromethyl)pyrazolo [1,5-a]pyridin-2- yl)-3,3- dimethylbutanamide | | | 4.03 | 394.4 |
| 76 | 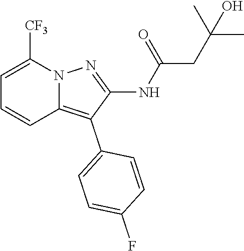 [SMILES: CC(C)(O)CC(=O)Nc1nn2c(C(F)(F)F)cccc2c1-c1ccc(F)cc1] | N-(3-(4- fluorophenyl)-7- (trifluoromethyl)pyrazolo [1,5-a]pyridin-2- yl)-3-hydroxy-3- methylbutanamide | | | 3.40 | 396.4 |
| 77 | 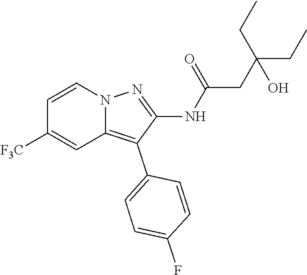 [SMILES: CCC(O)(CC)CC(=O)Nc1nn2ccc(C(F)(F)F)cc2c1-c1ccc(F)cc1] | 3-ethyl-N-(3-(4- fluorophenyl)-5- (trifluoromethyl)pyrazolo [1,5-a]pyridin-2- yl)-3- hydroxypentanamide | | | 3.79 | 424.4 |
| 78 | 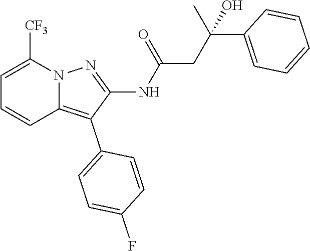 [SMILES: C[C@](O)(CC(=O)Nc1nn2c(C(F)(F)F)cccc2c1-c1ccc(F)cc1)c1ccccc1] | (S)-N-(3-(4- fluorophenyl)-7- (trifluoromethyl)pyrazolo [1,5-a]pyridin-2- yl)-3-hydroxy-3- phenylbutanamide | | | 3.90 | 458.4 |
| 79 | 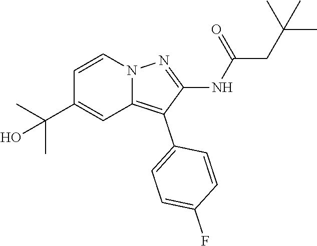 [SMILES: CC(C)(C)CC(=O)Nc1nn2ccc(C(C)(C)O)cc2c1-c1ccc(F)cc1] | N-(3-(4- fluorophenyl)-5-(2- hydroxypropan-2- yl)pyrazolo[1,5- a]pyridin-2-yl)-3,3- dimethylbutanamide | | | 3.30 | 384.4 |
| 80 | 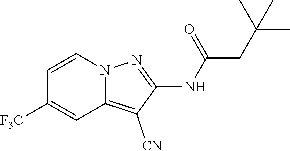 [SMILES: CC(C)(C)CC(=O)Nc1nn2ccc(C(F)(F)F)cc2c1C#N] | N-(3-cyano-5- (trifluoromethyl)pyrazolo [1,5-a]pyridin-2- yl)-3,3- dimethylbutanamide | | | 3.55 | 324.4 |
| 81 | 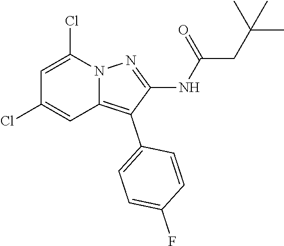 [SMILES: CC(C)(C)CC(=O)Nc1nn2c(Cl)cc(Cl)cc2c1-c1ccc(F)cc1] | N-(5,7-dichloro-3-(4- fluorophenyl)pyrazolo [1,5-a]pyridin-2-yl)- 3,3- dimethylbutanamide | | | 4.18 | 394.0 |
| 82 | 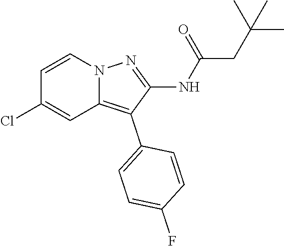 [SMILES: CC(C)(C)CC(=O)Nc1nn2ccc(Cl)cc2c1-c1ccc(F)cc1] | N-(5-chloro-3-(4- fluorophenyl)pyrazolo [1,5-a]pyridin-2-yl)- 3,3- dimethylbutanamide | | | 3.87 | 360.4 |
| 83 | 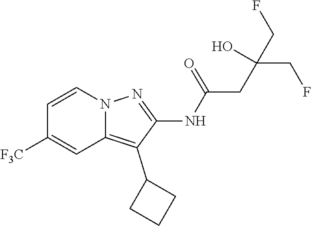 [SMILES: O=C(CC(O)(CF)CF)Nc1nn2ccc(C(F)(F)F)cc2c1C1CCC1] | N-(3-cyclobutyl-5- (trifluoromethyl) pyrazolo[1,5-a]pyridin-2- yl)-4-fluoro-3- (fluoromethyl)-3- hydroxybutanamide | | | 3.50 | 392.4 |
| 84 | 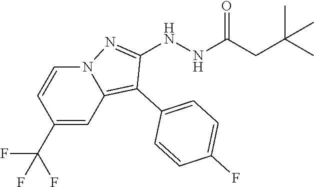 [SMILES: CC(C)(C)CC(=O)NNc1nn2ccc(C(F)(F)F)cc2c1-c1ccc(F)cc1] | N′-(3-(4- fluorophenyl)-5- (trifluoromethyl)pyrazolo [1,5-a]pyridin-2- yl)-3,3- dimethylbutanehydrazide | | | 3.95 | 409.2 |
| 85 | 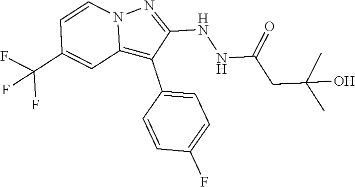 [SMILES: CC(C)(O)CC(=O)NNc1nn2ccc(C(F)(F)F)cc2c1-c1ccc(F)cc1] | N′-(3-(4- fluorophenyl)-5- (trifluoromethyl)pyrazolo [1,5-a]pyridin-2- yl)-3-hydroxy-3- methylbutanehydrazide | | | 3.78 | 411.2 |
| 86 | 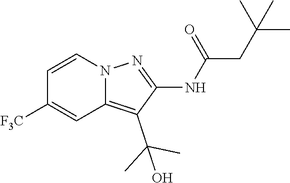 [SMILES: CC(C)(C)CC(=O)Nc1nn2ccc(C(F)(F)F)cc2c1C(C)(C)O] | N-(3-(2- hydroxypropan-2-yl)- 5- (trifluoromethyl) pyrazolo[1,5-a]pyridin-2- yl)-3,3- dimethylbutanamide | | | 3.32 | (M − H) = 356.4 Negative ionization |
| 87 | 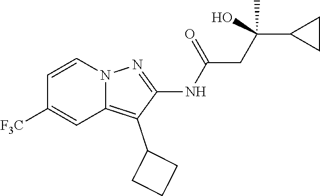 [SMILES: C[C@@](O)(CC(=O)Nc1nn2ccc(C(F)(F)F)cc2c1C1CCC1)C1CC1] | (R)-N-(3-cyclobutyl- 5- (trifluoromethyl) pyrazolo[1,5-a]pyridin-2- yl)-3-cyclopropyl-3- hydroxybutanamide | | | 3.70 | 382.4 |
| 88 | 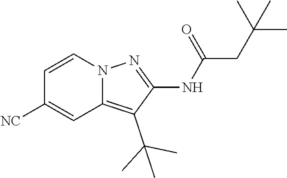 [SMILES: CC(C)(C)CC(=O)Nc1nn2ccc(C#N)cc2c1C(C)(C)C] | N-(3-(tert-butyl)-5- cyanopyrazolo[1,5- a]pyridin-2-yl)-3,3- dimethylbutanamide | | | 3.47 | 313.3 |
| 89 | 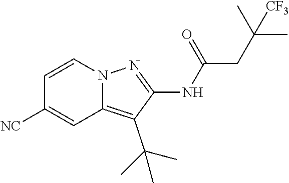 [SMILES: CC(C)(C)c1c(NC(=O)CC(C)(C)C(F)(F)F)nn2ccc(C#N)cc12] | N-(3-(tert-butyl)-5- cyanopyrazolo[1,5- a]pyridin-2-yl)-4,4,4- trifluoro-3,3- dimethylbutanamide | | | 3.62 | 367.2 |
| 90 | 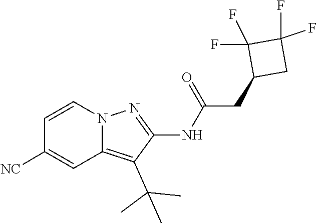 [SMILES: CC(C)(C)c1c(NC(=O)C[C@@H]2CC(F)(F)C2(F)F)nn2ccc(C#N)cc12] | (R)-N-(3-(tert-butyl)- 5-cyanopyrazolo[1,5- a]pyridin-2-yl)-2- (2,2,3,3- tetrafluorocyclobutyl) acetamide | | | 3.44 | 383.2 |
| 91 | 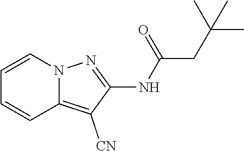 [SMILES: CC(C)(C)CC(=O)Nc1nn2ccccc2c1C#N] | N-(3- cyanopyrazolo [1,5- a]pyridin-2-yl)-3,3- dimethylbutanamide | | | 2.96 | 257.2 |
| 92 | 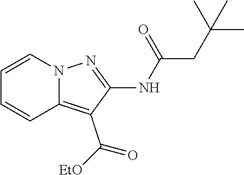 [SMILES: CCOC(=O)c1c(NC(=O)CC(C)(C)C)nn2ccccc12] | ethyl 2-(3,3- dimethylbutanamido) pyrazolo[1,5- a]pyridine-3- carboxylate | | | 3.60 | 304.4 |
| 93 | 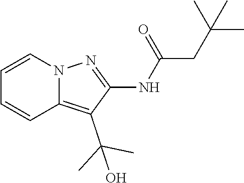 [SMILES: CC(C)(C)CC(=O)Nc1nn2ccccc2c1C(C)(C)O] | N-(3-(2- hydroxypropan-2- yl)pyrazolo[1,5- a]pyridin-2-yl)-3,3- dimethylbutanamide | | | 2.79 | (M − H) = 288.4 Negative ionization |
| 94 | 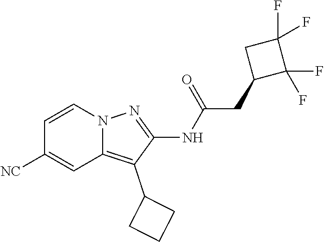 [SMILES: N#Cc1ccn2nc(NC(=O)C[C@H]3CC(F)(F)C3(F)F)c(C3CCC3)c2c1] | (S)-N-(5-cyano-3- cyclobutylpyrazolo[1, 5-a]pyridin-2-yl)-2- (2,2,3,3- tetrafluorocyclobutyl) acetamide | | | 3.52 | 381.2 |
| 95 | 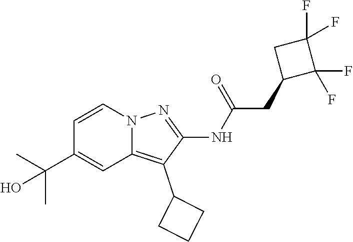 [SMILES: CC(C)(O)c1ccn2nc(NC(=O)C[C@H]3CC(F)(F)C3(F)F)c(C3CCC3)c2c1] | (S)-N-(3-cyclobutyl- 5-(2-hydroxypropan- 2-yl)pyrazolo [1,5- a]pyridin-2-yl)-2- (2,2,3,3- tetrafluorocyclobutyl) acetamide | | | 3.23 | 414.4 |
| 96 | 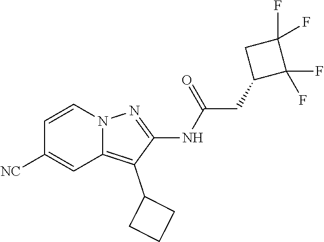 [SMILES: N#Cc1ccn2nc(NC(=O)C[C@@H]3CC(F)(F)C3(F)F)c(C3CCC3)c2c1] | (R)-N-(5-cyano-3- cyclobutylpyrazolo[1, 5-a]pyridin-2-yl)-2- (2,2,3,3- tetrafluorocyclobutyl) acetamide | | | 3.50 | 381.2 |
| 97 | 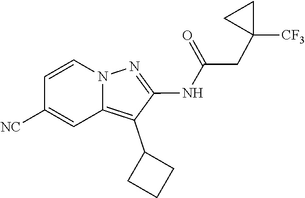 [SMILES: N#Cc1ccn2nc(NC(=O)CC3(C(F)(F)F)CC3)c(C3CCC3)c2c1] | N-(5-cyano-3- cyclobutylpyrazolo[1, 5-a]pyridin-2-yl)-2- (1- (trifluoromethyl) cyclopropyl)acetamide | | | 3.47 | 363.3 |
| 98 | 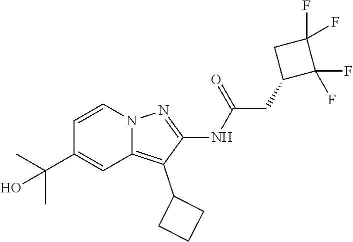 [SMILES: CC(C)(O)c1ccn2nc(NC(=O)C[C@@H]3CC(F)(F)C3(F)F)c(C3CCC3)c2c1] | (R)-N-(3-cyclobutyl- 5-(2-hydroxypropan- 2-yl)pyrazolo[1,5- a]pyridin-2-yl)-2- (2,2,3,3- tetrafluorocyclobutyl) acetamide | | | 3.25 | 414.1 |

### Pharmaceutical Compositions

Embodiments herein are directed to pharmaceutical compositions comprising a therapeutically effective amount of a compound described herein or acceptable salts thereof, such as a compound of Formula 1 or 2 or Table 1, or pharmaceutically acceptable salts thereof. Pharmaceutical formulations containing such compounds and a suitable carrier can be in various forms including, but not limited to, solids, solutions, powders, fluid emulsions, fluid suspensions, semi-solids, and dry powders including an effective amount of a compound of the invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, antioxidants, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Oilman's, The Pharmaceutical Basis of Therapeutics, 6th Edition, MacMillan Publishing Co., New York (1980) both of which are hereby incorporated by reference in their entireties can be consulted.

A subject composition may be formulated for any desirable route of delivery including, but not limited to, parenteral, intravenous, intradermal, subcutaneous, oral, inhalative, transdermal, topical, transmucosal, rectal, interacisternal, intravaginal, intraperitoneal, buccal, and intraocular.

Parenteral, intradermal or subcutaneous formulations may be sterile injectable aqueous or oleaginous suspensions or solutions. Acceptable vehicles, solutions, suspensions and solvents may include, but are not limited to, water or other sterile diluent; saline; Ringer's solution; sodium chloride; fixed oils such as mono- or diglycerides; fatty acids such as oleic acid; polyethylene glycols; glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. A parenteral preparation may be enclosed in ampoules, disposable syringes or multiple dose vials made of glass or plastic.

Pharmaceutical compositions suitable for injectable use may include sterile aqueous solutions or dispersions and sterile powders for the extemporaneous preparation of sterile injectable solutions or dispersion. For intravenous administration, suitable carriers include, but are not limited to, saline, bacteriostatic water, CREMOPHOR EL® (BASF, Parsippany, NJ) or phosphate buffered saline (PBS). The solvent or dispersion medium may contain, for example, water, ethanol, polyol (for example, glycerol, propylene glycol, and liquid polyethylene glycol, and the like), and suitable mixtures thereof. Proper fluidity can be maintained, for example, by the use of a coating such as lecithin, by the maintenance of the required particle size in the case of dispersion and by the use of surfactants. Preventing growth of microorganisms can be achieved by various antibacterial and antifungal agents, for example, parabens, chlorobutanol, phenol, ascorbic acid, thimerosal, and the like. The composition may also include isotonic agents such as, for example, sugars; polyalcohols such as mannitol; sorbitol; or sodium chloride. Prolonged absorption of injectable compositions can be enhanced by addition of an agent that delays absorption, such as, for example, aluminum monostearate or gelatin.

Oral compositions may include an inert diluent or an edible carrier. They may be enclosed in gelatin capsules or compressed into tablets. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In addition to oral or injected administration, systemic administration may be by transmucosal or transdermal means. For transmucosal or transdermal administration, penetrants may be used. Such penetrants are generally known in the art and include, for example, detergents, bile salts, and fusidic acid derivatives. Transdermal administration may include a bioactive agent and may be formulated into ointments, salves, gels, or creams as generally known in the art. Transmucosal administration may be accomplished through the use of nasal sprays or suppositories.

A subject compound may be administered in a therapeutically effective amount, according to an appropriate dosing regimen. As understood by a skilled artisan, an exact amount required may vary from subject to subject, depending on a subject's species, age and general condition, the severity of the infection, the particular agent(s) and the mode of administration. In some embodiments, about 0.001 mg/kg to about 50 mg/kg, of the pharmaceutical composition based on the subject's body weight is administered, one or more times a day, to obtain the desired therapeutic effect. In other embodiments, about 0.01 mg/kg to about 25 mg/kg, of the pharmaceutical composition based on the subject's body weight is administered, one or more times a day, to obtain the desired therapeutic effect.

A total daily dosage of a subject compound can be determined by the attending physician within the scope of sound medical judgment. A specific therapeutically effective dose level for any particular patient or subject will depend upon a variety of factors including the disorder being treated and the severity of the disorder; the activity of the specific compound employed; the specific composition employed; the age, body weight, general health, sex and diet of the patient or subject; the time of administration, route of administration, and rate of excretion of the specific compound employed; the duration of the treatment; drugs used in combination or coincidental with the specific compound employed; and other factors well known in the medical arts.

### Methods of Use

Embodiments are directed to methods of treating a disorder in a subject in need thereof comprising administering to the subject a therapeutically effective amount of a subject compound of Formula 1 or Formula 2 or Table 1. In some embodiments, the disorder is selected from epilepsy, neonatal spasms, pain, migraine, a disorder of neurotransmitter release, a smooth muscle contractility disorder, a dyskinesia, dystonia, mania, a hearing disorder, neuropathic pain, inflammatory pain, persistent pain, cancer pain, postoperative pain, anxiety, substance abuse, schizophrenia, a bladder disorder, a vasculature disorder, tinnitus, benign familial neonatal seizures, epilepsy, neurological disease via reduced basal M-current (and subsequent neuronal hyperexcitability), sensorineural hearing impairment, intellectual disability, epileptic encephalopathy, treatment-resistant epilepsy, cortical atrophy, neurological impairment, infantile spasms with hypsarrhythmia, myoclonic-tonic seizures, myoclonic seizures, tonic seizures, absence and focal-onset seizures with impaired awareness, congenital neurological disorder with intellectual disability or epileptic encephalopathy, benign familial neonatal convulsions, severe epileptic encephalopathies, congenital neurodevelopmental disorder with phenotypes of nonsyndromic intellectual disability or epileptic encephalopathy, neonatal spasms, neonatal seizures, epilepsy, benign familial neonatal epilepsy, epileptic encephalopathy, benign familial neonatal convulsions type 1, benign familial neonatal seizures 1, neonatal seizures associated with hypoxic-ischemic injury, epileptic spasms, epileptic encephalopathy, early infantile epileptic encephalopathy 7, early infantile epileptic encephalopathy with delayed psychomotor development, generalized tonic seizures, abnormal globus pallidus morphology, apnea, cerebral edema, dystonia, facial erythema, muscular hypotonia, febrile seizures, hypoplasia of the corpus callosum, hypsarrhythmia, focal clonic seizure, generalized tonic-clonic seizures, myokymia, spastic tetraparesis, myokymia, and combinations thereof. In embodiments, such compound may be administered in a pharmaceutical composition as described herein.

Without wishing to be bound by theory, various Kv7 channels have been shown to be diversely expressed throughout the body. For example, Kv7.1 is highly expressed in the heart, cardiac myocytes, renal proximal tubules, gastrointestinal tract, colonic crypt cells, pancreatic acinar cells, thyroid cells and airway epithelium. Kv7.2 and Kv7.3 are robustly expressed in central, peripheral, and sensory neurons. Kv7.4 is highly expressed in the cochlea of the inner ear and cardiac mitochondria. Kv7.5 is expressed in neurons and skeletal muscle. Channel subunits Kv7.1, Kv7.4 and Kv7.5 are highly expressed in vascular and non-vascular smooth muscle. Accordingly, activating or modulating particular Kv7 subunits will be effective in treating a variety of disorders associated with the nervous, cardiovascular, urogenital, digestive, gynecological, and respiratory systems.

The KCNQ1-5 genes encode the five Kv7 potassium channel subunits 1-5, respectively. A functional Kv7 potassium channel can be assembled using a combination of these five subunits arranged as homotetramers or heterotetramers. Mutations in the genes encoding each Kv7 channel have been associated with human diseases. Accordingly, activators or modulators of mutated channels can be effective to alleviate the symptoms associated with the disease.

In most neurons of the central nervous system (CNS) as well as peripheral nerves, the dominant Kv7 subunits are Kv7.2, Kv7.3, and Kv7.5. Kv7 activators can be used in the treatment of stroke and neuropathic pain. Inhibition of Kv7 channels facilitate synaptic plasticity and can be used as cognition enhancers. Kv7 activators, rather than blockers, may prevent cognitive dysfunction in Alzheimer's disease (AD). Kv7 modulators can also be used in the treatment of schizophrenia, drug abuse, and anxiety. Kv7.2/7.3 in the nucleus accumbens is altered by chronic alcohol intake. Kv7 activators possess antidepressant activity, possibly via the potentiation of resilience (capacity to cope with stress) mechanisms.

Mutations in KCNQ2 and KCNQ3 genes are associated with an inherited benign form of epilepsy of the newborn. Variants in the KCNQ2 gene are responsible for a wide spectrum of phenotypes characterized by hyperexcitability, ranging from mild and self-limiting epilepsy [benign familial neonatal epilepsy (BFNE)] to severe epileptic encephalopathy with cognitive impairment, neuroradiological alterations, and pharmacoresistant seizures [neonatal epileptic encephalopathy (NEE)]. Variants in KCNQ3 have also been described but are associated only with BFNE. Such heterogeneity is partly explained by different mutations affecting different domains of Kv7.2 channels. Alteration of Kv7 channel activity is also involved in brain ischemic injury, age-related impairment of memory, stress-related dysfunction of the neuroendocrine system, addiction, and neuronal differentiation. Accordingly, Kv7 channel activators can be used to treat epilepsy, partial-onset seizures, and pharmacoresistant seizures, i.e. NEE. Modulation of Kv7 channels can treat many other diseases driven by neuronal hyperexcitability, such as neuropathic pain, ischemia, and schizophrenia. More recently, Kv7.3/7.5 dysfunction has been associated with autism.

Embodiments of the present invention relate to a method of treating a disorder associated with a Kv7 potassium channel comprising administering a therapeutically effective amount of a compound as described herein or a compound of Formula 1 or Formula 2 or Table 1 as described herein, or a pharmaceutically acceptable salt thereof, to a subject in need thereof. The disorder is a disorder associated with the nervous, cardiovascular, urogenital, digestive, or respiratory systems.

In some embodiments, the nervous system disorders are selected from the group consisting of epilepsy, epileptic spasms, neonatal spasms, neonatal seizures, benign familial neonatal epilepsy (BFNE), neonatal epileptic encephalopathy (NEE), focal seizures, focal epilepsy, myoclonic seizures, tonic and clonic seizures, tonic-clonic (grand mal) seizures, partial-onset seizures, pharmacoresistant seizures, pain, migraine, a disorder of neurotransmitter release, early infantile epileptic encephalopathy (EIEE, Ohtahara syndrome) with delayed psychomotor development, generalized tonic seizures, abnormal globus pallidus morphology, apnea, cerebral edema, a dyskinesia, dystonia, facial erythema, muscular hypotonia, febrile seizures, hypoplasia of the corpus callosum, hypsarrhythmia, focal clonic seizure, generalized tonic-clonic seizures, infantile spasms (West syndrome), Doose syndrome (myoclonic astatic epilepsy of childhood), benign Rolandic epilepsy (BRE), Rasmussen syndrome, Lennox-Gastaut syndrome, electrical status epilepticus of sleep (ESES), Sturge-Weber syndrome, juvenile myoclonic epilepsy, Dravet syndrome, myokymia, spastic tetraparesis, myokymia, mania, a hearing disorder, neuroradiological alterations, deafness, neuropathic pain, inflammatory pain, persistent pain, cancer pain, postoperative pain, brain ischemic injury, age-related impairment of memory, stress-related dysfunction of the neuroendocrine system, anxiety, depression, substance abuse, addiction, chronic alcohol intake, schizophrenia, autism, amyotrophic lateral sclerosis, Alzheimer's disease, tinnitus, and combinations thereof.

Compounds described herein have been shown to activate subtypes of the Kv7 potassium channel family. Mutations in the KCNQ2 or KCNQ3 genes, which encodes the Kv7.2 and Kv7.3 potassium channel subunits respectively, result in a range of epilepsy disorders. Embodiments herein are directed to methods of treating a disorder associated with a mutation in a KCNQ2 subunit comprising administering a therapeutically effective amount of a compound as described herein or a compound of Formula 1 or Formula 2 or Table 1 as described herein, or a pharmaceutically acceptable salt thereof, to a subject in need thereof. Embodiments herein are directed to methods of treating a disorder associated with a mutation in a KCNQ3 subunit comprising administering a therapeutically effective amount of a compound as described in Formula 1 or Formula 2 or Table 1, or any other compound as described herein, or a pharmaceutically acceptable salt thereof, to a subject in need thereof. In embodiments, the disorder that may be treated by a compound disclosed herein includes, but is not limited to epilepsy, epileptic spasms, neonatal spasms, neonatal seizures, benign familial neonatal epilepsy (BFNE), neonatal epileptic encephalopathy (NEE), focal seizures, focal epilepsy, myoclonic seizures, tonic and clonic seizures, tonic-clonic (grand mal) seizures, partial-onset seizures, pharmacoresistant seizures, pain, migraine, a disorder of neurotransmitter release, early infantile epileptic encephalopathy (EIEE, Ohtahara syndrome) with delayed psychomotor development, generalized tonic seizures, abnormal globus pallidus morphology, apnea, cerebral edema, a dyskinesia, dystonia, facial erythema, muscular hypotonia, febrile seizures, hypoplasia of the corpus callosum, hypsarrhythmia, focal clonic seizure, generalized tonic-clonic seizures, infantile spasms (West Syndrome), Doose syndrome (myoclonic astatic epilepsy of childhood), benign Rolandic epilepsy (BRE), Rasmussen syndrome, Lennox-Gastaut syndrome, electrical status epilepticus of sleep (ESES), Sturge-Weber syndrome, juvenile myoclonic epilepsy, Dravet syndrome, myokymia, spastic tetraparesis, myokymia, mania, a hearing disorder, neuroradiological alterations, deafness, neuropathic pain, inflammatory pain, persistent pain, cancer pain, postoperative pain, brain ischemic injury, age-related impairment of memory, stress-related dysfunction of the neuroendocrine system, anxiety, depression, substance abuse, addiction, chronic alcohol intake, schizophrenia, autism, amyotrophic lateral sclerosis, Alzheimer's disease, tinnitus, and combinations thereof. In embodiments, such compounds may be administered in a pharmaceutical composition as described herein.

Embodiments herein are directed to methods of treating a disorder associated with a KCNQ subunit comprising administering a therapeutically effective amount of a compound as described in Formula 1 or Formula 2 or Table 1, or any other compound as described herein, or a pharmaceutically acceptable salt thereof, to a subject in need thereof. In some embodiments, the KCNQ gene and the encoded Kv7 subunit are not mutated but activation of the subunit has a beneficial effect. Embodiments herein are directed to methods of treating a disorder associated with a KCNQ2 subunit comprising administering a therapeutically effective amount of a compound as described in Formula 1 or Formula 2 or Table 1, or any other compound as described herein, or a pharmaceutically acceptable salt thereof, to a subject in need thereof. Embodiments herein are directed to methods of treating a disorder associated with a KCNQ3 subunit comprising administering a therapeutically effective amount of a compound as described in paragraphs Formula 1 or Formula 2 or Table 1, or any other compound as described herein, or a pharmaceutically acceptable salt thereof, to a subject in need thereof.

In embodiments, such compound may be administered in a pharmaceutical composition as described herein.

Therapeutically effective amounts of the compounds disclosed herein range from about 0.1 mg to about 1000 mg. Such therapeutically effective amounts may be administered once a day or in equal, divided doses twice a day, three times a day, or four times a day.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### EXPERIMENTAL SECTION

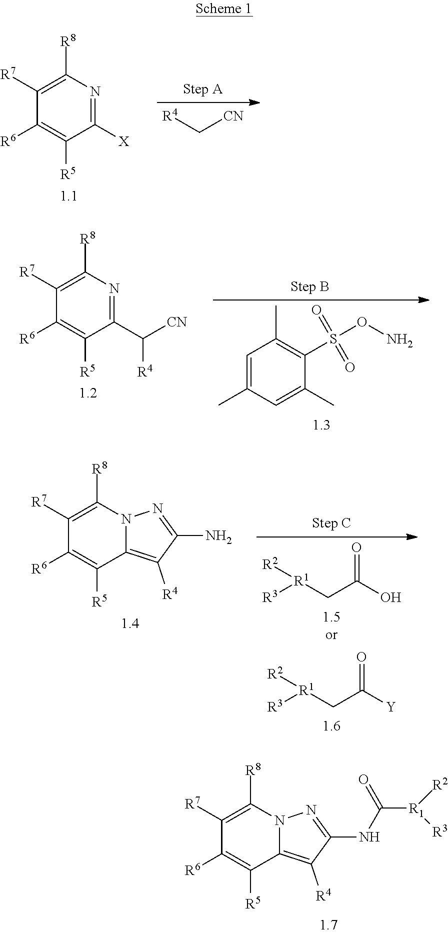

Scheme 1 shows a general methodology for the synthesis of pyrazolo[1,5-a]pyridin-2,3-yl amides 1.7 An appropriately substituted 2-halo-pyridine 1.1 is reacted with an appropriately substituted acetonitrile to afford 2-(1-cyanomethyl)pyridines 1.2. Pyridine 1.2 may be reacted with a suitable nitrogen donor reagent such as O-(mesitylsulfonyl) hydroxylamine 1.3, to generate the pyrazolo[1,5-a]pyridin-2,3-yl amines 1.4. Coupling of amines 1.4 with an appropriate carboxylic acid 1.5 under standard amide-forming conditions, or via reaction with an acyl halide (1.6, Y=Cl, F, Br), affords the target pyrazolo[1,5-a]pyridin-2,3-yl amides 1.7.

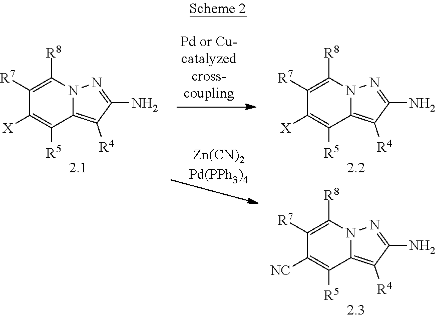

Scheme 2 shows a general methodology for the conversion of 5-halo-pyrazolo[1,5-a]pyridin-2,3-yl amines 2.1 (X=Cl, Br, I) to a variety of 5-substituted pyrazolo[1,5-a]pyridin-2,3-yl amines 2.2 by palladium, copper, or other transition-metal catalyzed cross-coupling reactions. Reactants 2.1 may also include 2-aminopyrazolo[1,5-a]pyridin-5-yl sulfonates (X=RSO₃). For example, an appropriately substituted halide or sulfonate 2.1 is treated with zinc(ll) cyanide and tetrakis(triphenylphosphine)palladium(0) to provide nitrile 2.3. A wide range of other transformations of 2.1 may be accomplished using conditions well-known in the art, including coupling reactions of alky-, aryl-, or heteroarylboronate reagents, alkenyl-, aryl-, or heteroaryltin reagents, substituted or unsubstituted alkenes, alkynes, amines, alcohols, ketones, and the like (see Magano J, Dunetz J R, Chem Rev 2011, 111, 2177-2250, and references cited therein).

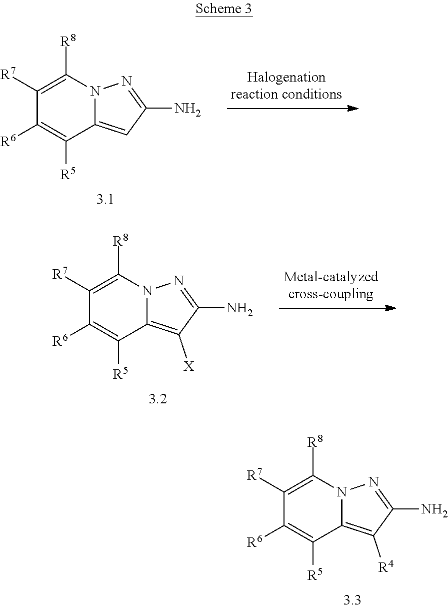

Scheme 3 shows a general methodology for the conversion of pyrazolo[1,5-a]pyridin-2,3-yl amines 3.1 into 3-halo-derivatives 3.2 (X=Cl, Br, I). Halides 3.2 may then be converted into a variety of 3-substituted pyrazolo[1,5-a]pyridin-2,3-yl amines 3.3 by palladium, copper, or other transition-metal catalyzed cross-coupling reactions. For example, an appropriately substituted halide 3.2 is treated with aryl-, heteroaryl- or alkylboronic acids or esters, together with dichloro-bis(triphenylphosphine)palladium(0) to provide 3.3 (R=alkyl, aryl, heteroaryl). A wide range of other transformations of 3.2 to 3.3 may be accomplished using conditions well-known in the art, as described in for Scheme 2 above.

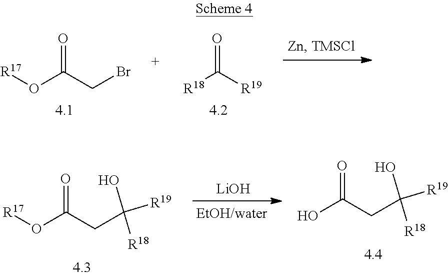

Scheme 4 shows a general methodology for the preparation of optionally substituted 3-hydroxypropanoic acids 4.4. Zinc-catalyzed condensation of bromo esters 4.1 with ketones or aldehydes 4.2 provides the 3-hydroxypropanoates 4.3. Standard hydrolysis conditions convert esters 4.3 to acids 4.4. Acids 4.4 are useful for amide bond-forming reactions as outlined in Scheme 1, Step C.

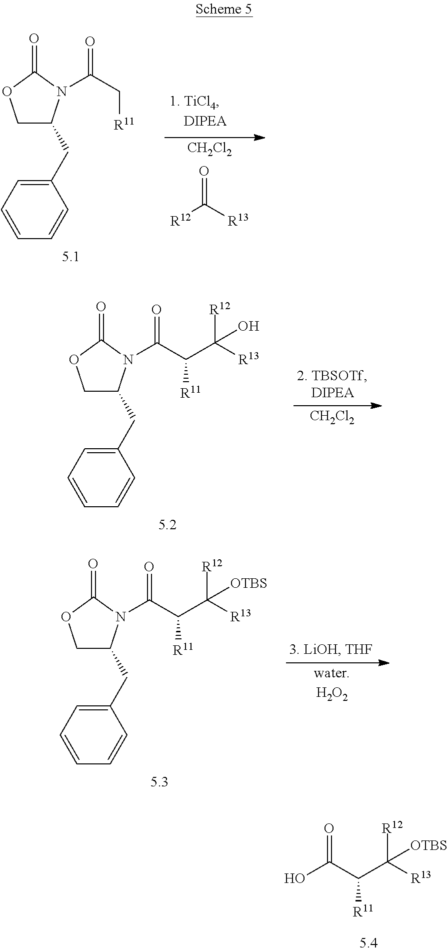

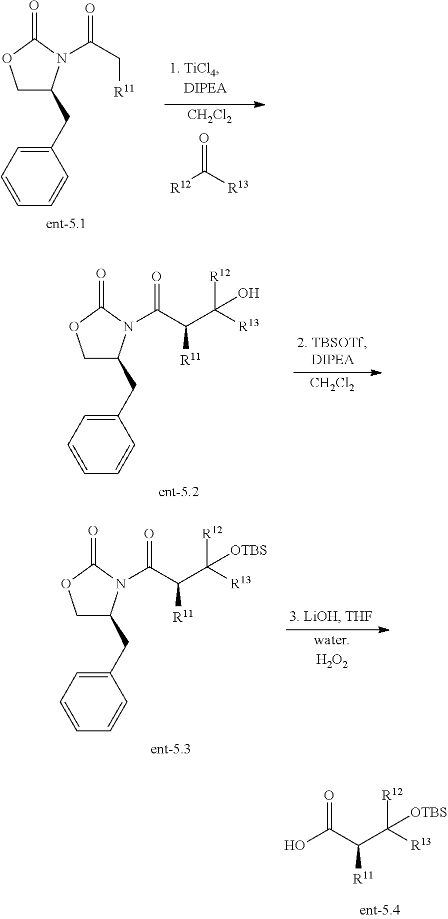

Scheme 5 describes a general synthetic method for the synthesis of chiral α-alkyl carboxylic acids that contain β-silyloxy ether protected acids 5.4 or ent-5.4. These optically active acids are used as the acid component in amide forming reaction (Step C of Scheme 1) to give β-tertiary alcohol amides. The diastereoselective bond construction via titanium enolate chemistry described by Evans and coworkers was used to condense chiral imide 5.1 or ent-5.1 with a ketone or other electrophile to give diastereomerically pure aldol adducts 5.2 or ent-5.2, respectively (see Evans, D A, Urpi, F, Somers, T C, Clark, J S, Bilodeau, M T, J. Am. Chem. Soc. 1990, 112, 8215-8216). The proper choice of chiral imide 5.1 will give rise to the desired absolute stereochemistry of the α-stereocenter in the carboxylic acids 5.4 or ent-5.4. Silyl ether protection of the aldol adducts 5.2 and ent-5.2 with tert-butyldimethylsilyltriflate and diisopropylethylamine gives the tert-butyldimethylsilyl ethers 5.3 and ent-5.3. Standard acyl oxazolidinone hydrolysis conditions using lithium hydroxide and hydrogen peroxide in tetrahydrofuran and water provides the desired acids 5.4 or ent-5.4 (see Evans, D A, Britton, T C, Ellman, J A, Tetrahedron Lett. 1987, 28 (49), 6141-6144). Proper selection of ketones or other electrophiles in the titanium enolate chemistry will give rise to appropriately substituted aldol adducts that vary in the nature of the R¹² and R¹³ groups. Changing the R¹¹ group of the starting imides 5.1 and ent-5.1 may be used to vary the size and nature of the R¹¹ group in the acids 5.4 or ent-5.4. This methodology allows for the synthesis of a wide range of optically active acids with absolute stereocontrol of the α-chiral center to the carbonyl of the carboxylic acid.

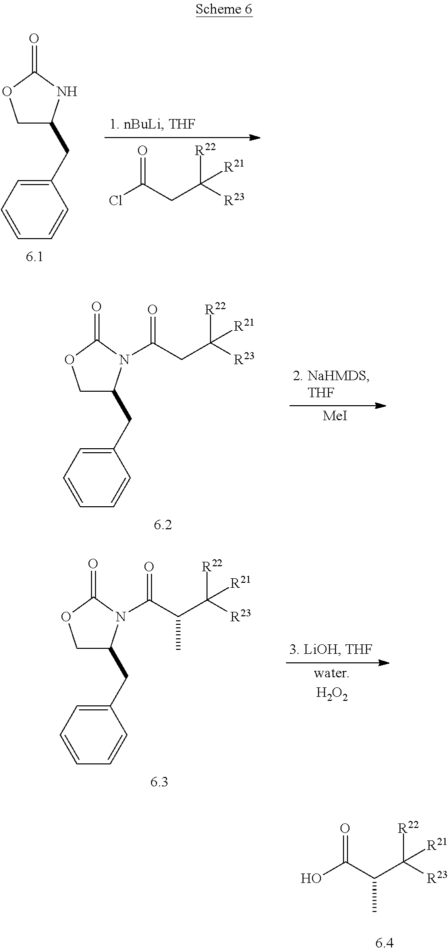

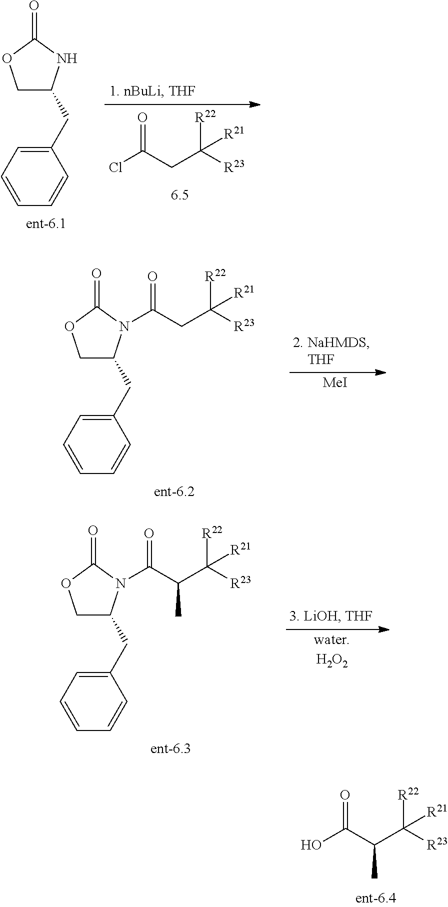

A general synthetic method for the synthesis of enantiomerically pure α-methyl-β-branched chiral carboxylic acids 6.4 or ent-6.4 is described in Scheme 6. Both of the enantiomerically pure oxazolidinones(S)-4-benzyloxazolidin-2-one 6.1 and (R)-4-benzyloxazolidin-2-one ent-6.1 are commercially available. These oxazolidinones may be readily acylated by deprotonation with n-butyllithium followed by reaction with acid chlorides 6.5 to give the chiral imides 6.2 and ent-6.2, respectively. There are a large number of commercially available acid chlorides 6.5 with wide variation about the R²¹, R²² and R²³ groups of this input. This allows for the rapid synthesis of chiral imides 6.2 and ent-6.2 that have differing substitution at the β-position to the exocyclic carbonyl group. The asymmetric alkylation reaction of chiral imide sodium enolates as developed by Evans may then be used to introduce a methyl group in a stereoselective fashion (see Evans, D A, Ennis, M D, Mathre, D J, J. Am. Chem. Soc. 1982, 104, 1737-1739). The sodium enolate of imide 6.2 may be produced by treatment of 6.2 with sodium hexamethyldisilazide in tetrahydrofuran. The resultant sodium enolate may then be stereoselectively methylated by the addition of methyl iodide. The pure and single diastereomers 6.3 and ent-6.3 may be isolated by silica gel column chromatography. Alternatively, the single diastereomers may be obtained by recrystallization of crystalline products 6.3 and ent-6.3. The well-known chiral auxiliary hydrolysis conditions as described above for Scheme 5 give the optically active α-methyl β-branched chiral carboxylic acids 6.4 or ent-6.4, respectively.

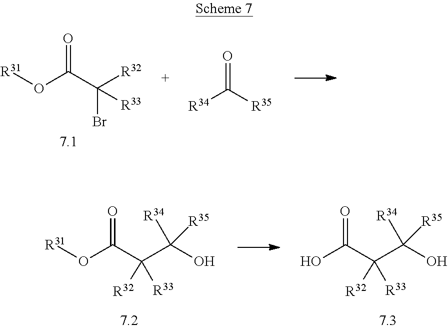

Scheme 7 shows a general methodology for the synthesis of 3-hydroxypropanoic acids such as 7.3. An appropriately substituted 2-bromoethanoic ester 7.1 is reacted with a ketone or aldehyde to afford 3-hydroxypropanoic esters 7.2. The ester group may be hydrolyzed to the corresponding acid by saponification to provide 3-hydroxypropanoic acids such as 7.3.

Scheme 7 shows a general methodology for the synthesis of 3-hydroxypropanoic acids such as 7.3. An appropriately substituted 2-bromoethanoic ester 7.1 is reacted with a ketone or aldehyde to afford 3-hydroxypropanoic esters 7.2. The ester group may be hydrolyzed to the corresponding acid by saponification to provide 3-hydroxypropanoic acids such as 7.3.

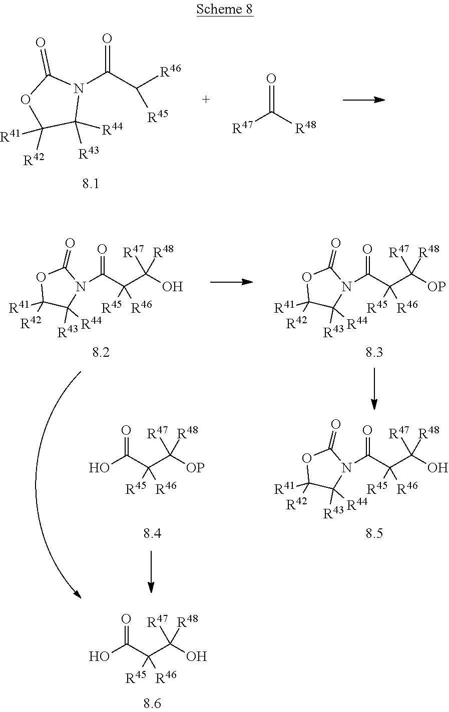

Scheme 8 depicts additional methods for the preparation of optionally substituted 3-hydroxypropanoic acids. An appropriately substituted 3-acetyloxazolidin-2-one 8.1 is reacted with a ketone or aldehyde to afford 3-(3-hydroxypropanoyl) oxazolidin-2-ones 8.2. The hydroxyl group is functionalized with a protecting group to provide diastereomers 8.3 that are separable by silica gel chromatography. Each diastereomer 8.3 is then reacted in a two-step sequence, in either order, of hydroxyl group deprotection and oxazolidinone cleavage to provide 3-hydroxypropanoic acids such as 8.6. In addition, acyloxazolidinones 8.2 may be hydrolyzed directly to hydroxyacids 8.6 by standard conditions.

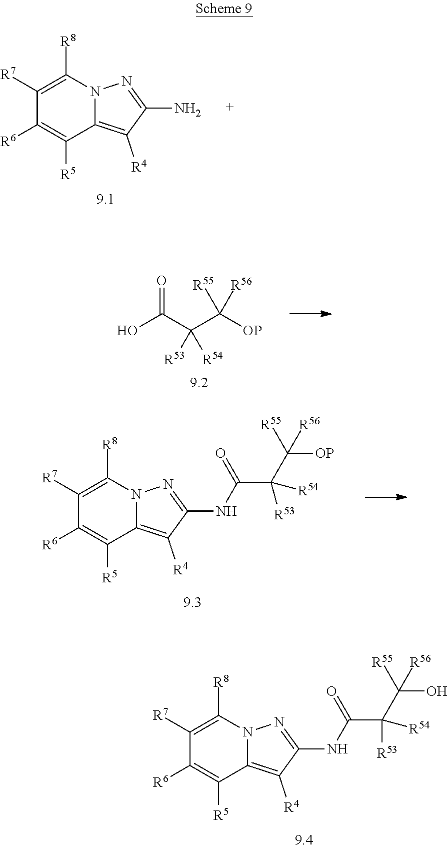

Scheme 9 describes methods that can be employed prepare pyrazolo[1,5-a]pyridin-2,3-yl amides substituted with hydroxyl-containing acyl groups such as 9.4. Appropriately substituted pyrazolo[1,5-a]pyridin-2,3-yl amines 9.1 may be coupled with protected alcohol derivatives such as 9.2 to afford the corresponding amides 9.3. The alcohol protecting group can be removed via several methods, for example removal of silyl groups using tetrabutylammonium fluoride, to provide alcohol-containing pyrazolo[1,5-a]pyridin-2,3-yl amides such as such as 9.4.

### Synthetic Methods

### Section 1. Representative Procedures for the Preparation of pyrazolo[1,5-a]pyridine-2,3-yl amines (Compounds 1.4, Scheme 1)

### Method 1:

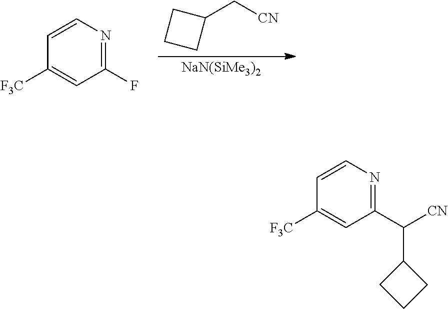

Step A. Preparation of 2-cyclobutyl-2-(4-(trifluoromethyl)pyridin-2-yl)acetonitrile. To a solution of 2-fluoro-4-(trifluoromethyl)pyridine (0.50 g, 3.02 mmol) and 2-cyclobutylacetonitrile (0.29 mL, 3.02 mmol) in toluene (3 mL) at 0° C. was added sodium hexamethyldisilazide solution (6.1 mL, 1 M in THF, 6.1 mmol) dropwise. The mixture was allowed to warm to room temperature slowly and was stirred overnight. The mixture was quenched with saturated aqueous ammonium chloride and extracted with EtOAc. Column chromatography (0-50% EtOAc/hex) provided 2-cyclobutyl-2-(4-(trifluoromethyl)pyridin-2-yl)acetonitrile (0.39 g, 1.62 mmol, 54% yield). MS (ESI) m/z 241.2 (MH⁺). ¹H NMR (CDCl₃): δ 8.78 (d, J=4.8 Hz, 1H), 7.57 (s, 1H), 7.49 (d, 4.8 Hz, 1H), 4.09 (d, J=7.6 Hz, 1H), 2.99-2.93 (m, 1H), 2.12-2.02 (m, 4H), 1.97-1.88 (m, 2H).

The following 2-(pyridin-2-yl)acetonitriles were prepared using the general procedure described in Section 1, Method 1, Step A, with appropriate starting materials:
2-Cyclobutyl-2-(pyridin-2-yl)acetonitrile
2-(4-Chloropyridin-2-yl)-2-cyclobutylacetonitrile
2-(4-Bromopyridin-2-yl)-2-cyclobutylacetonitrile
2-(4-(Trifluoromethyl)pyridin-2-yl)acetonitrile
2-(4-Fluorophenyl)-2-(4-(trifluoromethyl)pyridin-2-yl)acetonitrile
2-(6-Bromopyridin-2-yl)-2-cyclobutylacetonitrile
2-Cyclobutyl-2-(4-methoxypyridin-2-yl)acetonitrile

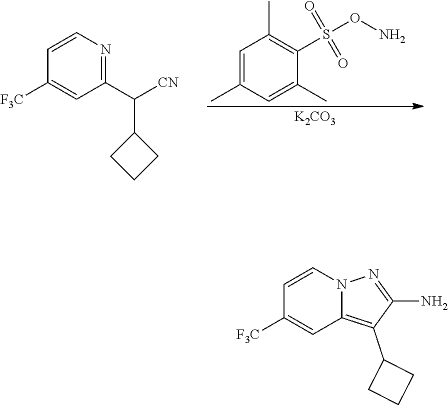

Step B. Preparation of 3-cyclobutyl-5-(trifluoromethyl)pyrazolo[1,5-a]pyridin-2-amine. To O-(mesitylsulfonyl) hydroxylamine (1.2 g, 5.8 mmol, prepared by procedure described in Org. Proc. Res. Dev. 2009, 13, 263-267) in dichloromethane (30 mL) at 0° C. was added 2-cyclobutyl-2-(4-(trifluoromethyl)pyridin-2-yl)acetonitrile (0.93 g, 3.9 mmol). After 10 min the reaction mixture was allowed to warm to room temperature and stirred overnight. Solvent was removed in vacuo. The residue was dissolved in MeOH and K₂CO₃ was added. After 2 h, MeOH was removed in vacuo. The residue was taken up in EtOAc and H₂O and the layers were separated. The aqueous phase was extracted with EtOAc and the combined organic layers were dried over Na₂SO₄ and concentrated. Column chromatography (0-50% EtOAc/hexane) provided 0.37 g (37% yield) of 3-cyclobutyl-5-(trifluoromethyl)pyrazolo[1,5-a]pyridin-2-amine. MS (ESI) m/z 256.0 (MH⁺). ¹H NMR (CDCl₃): δ 8.18 (d, J=7.2 Hz, 1H), 7.54 (s, 1H), 6.59 (d, J=7.2 Hz), 3.97 (s, 2H), 3.60-3.56 (m, 1H), 2.45-2.34 (m, 4H), 2.12-2.05 (m, 1H), 1.98-1.95 (m, 1H).

The following pyrazolo[1,5-a]pyridin-2-amines were prepared using the general procedure described in Section 1, Method 1, Step B, with appropriate starting materials.
3-Cyclobutylpyrazolo[1,5-a]pyridin-2-amine
5-Chloro-3-cyclobutylpyrazolo[1,5-a]pyridin-2-amine
5-Bromo-3-cyclobutylpyrazolo[1,5-a]pyridin-2-amine
5-(Trifluoromethyl)pyrazolo[1,5-a]pyridin-2-amine
7-Bromo-3-cyclobutylpyrazolo[1,5-a]pyridin-2-amine

3-(4-Fluorophenyl)-5-(trifluoromethyl)pyrazolo[1,5-a]pyridin-2-amine: A modified procedure was used for isolation of title compound. After the reaction mixture of starting material and MSH in dichloromethane was stirred overnight, the precipitate that formed in the reaction mixture was removed by filtration. Then the filtrate was concentrated in vacuo. The residue was taken up in ether and washed with 0.50 M KOH two times. The organic layer was dried (Na₂SO₄) and concentrated. Column chromatography (0-50% EtOAc/hexane) provided a 37% yield of 3-(4-fluorophenyl)-5-(trifluoromethyl)pyrazolo[1,5-a]pyridin-2-amine.

### Method 2: Exemplary Procedure for Cross Coupling of Halo-Substituted pyrazolo[1,5-a]pyridin-2-amines (Scheme 2)

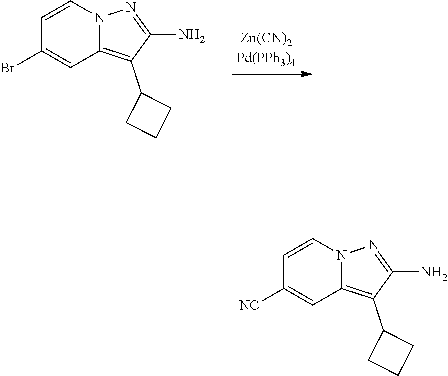 [SMILES: Nc1nn2ccc(Br)cc2c1C1CCC1 ; N#Cc1ccn2nc(N)c(C3CCC3)c2c1]

Preparation of 2-amino-3-cyclobutylpyrazolo[1,5-a]pyridine-5-carbonitrile. Nitrogen gas was bubbled into a mixture of 5-bromo-3-cyclobutylpyrazolo[1,5-a]pyridin-2-amine (0.49 g, 1.8 mmol), zinc cyanide (0.21 g, 1.8 mmol), and tetrakis(triphenylphosphine) palladium[0] (0.43 g, 0.37 mmol) in N-methylpyrrolidinone (10 mL) for 10 min. The mixture was then heated to 110° C. in a sealed vial. After 5 h the mixture was diluted with EtOAc and washed with water three times. The organic extract was dried over Na₂SO₄ and concentrated. Column chromatography (0-100% EtOAc/hexane) provided 0.27 g (70% yield) of the title compound. MS (ESI) m/z 213.2 (MH⁺). ¹H NMR (CDCl₃): δ 8.14 (dd, J=0.8, 7.2 Hz, 1H), 7.65 (dd, J=0.8, 2.0 Hz, 1H), 6.55 (dd, J=2, 7.2 Hz, 1H), 4.00 (s, 2H), 3.60-3.53 (m, 1H), 2.50-2.31 (m, 4H), 2.20-2.05 (m, 1H), 1.98-1.95 (m, 1H).

### Method 3: Exemplary Procedures for Iodination and Subsequent Cross Coupling of 3-unsubstituted pyrazolo[1,5-a]pyridin-2-amines (Scheme 3)

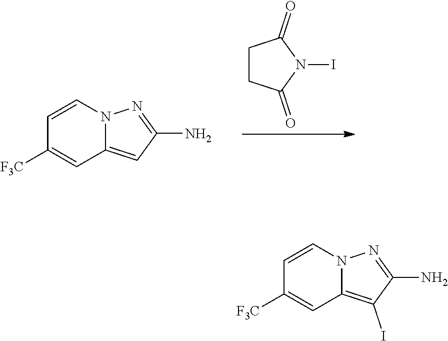 [SMILES: Nc1cc2cc(C(F)(F)F)ccn2n1 ; O=C1CCC(=O)N1I ; Nc1nn2ccc(C(F)(F)F)cc2c1I]

Step A. Preparation of 3-iodo-5-(trifluoromethyl)pyrazolo[1,5-a]pyridin-2-amine. 5-(Trifluoromethyl)pyrazolo[1,5-a]pyridin-2-amine (165 mg, 0.82 mmol) was dissolved in CH₃CN (3.3 mL) and cooled to 0° C. To this solution was added N-iodosuccinimide (146 mg, 0.82 mmol) and the solution was warmed to room temperature and stirred for 2 h. The reaction mixture was diluted with EtOAc, washed sequentially with H₂O and brine, dried (Na₂SO₄), filtered and concentrated in vacuo. Purification by column chromatography (0-40% EtOAc/hexanes) provided the title compound (154 mg, 67%). MS (ESI) 280.0/282.0 (MH⁺).

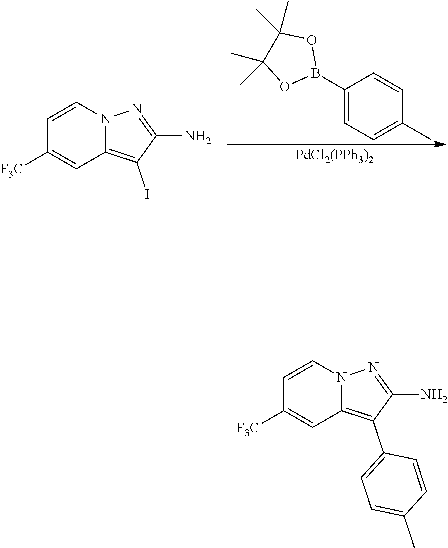

Step B. Preparation of 3-(p-tolyl)-5-(trifluoromethyl)pyrazolo[1,5-a]pyridin-2-amine. To a pressure tube was combined 3-iodo-5-(trifluoromethyl)pyrazolo[1,5-a]pyridin-2-amine (75 mg, 0.23 mmol), 4,4,5,5-tetramethyl-2-(p-tolyl)-1,3,2-dioxaborolane (100 mg, 0.46 mmol), 1:1 Dioxane/H₂O (1.5 mL), solid Na₂CO₃ (24 mg, 0.23 mmol) and dichloro-bis(triphenylphosphine) palladium[0] (3 mg, 0.005 mmol) and the tube was sealed and heated at 150° C. for 1 h. The mixture was cooled, the solution was diluted with EtOAc, washed with brine, the organic layer was dried (Na₂SO₄), filtered and concentrated in vacuo. Purification by column chromatography (0-30% EtOAc/hexanes) provided the title compound (51 mg, 76%). NMR (CDCl₃) δ 8.26 (d, J=7.2 Hz, 1H), 7.66 (s, 1H), 7.40 (d, J=7.6 Hz, 2H), 7.32 (d, J=7.6 Hz, 2H), 6.71 (dd, J=2.0, 8.0 Hz, 1H), 4.22 (s, 2H), 2.42 (s, 3H). MS (ESI) m/z 278.0 (MH⁺).

The following pyrazolo[1,5-a]pyridin-2-amines were prepared using the general procedure described in Section 1, Method 3, Step B, with appropriate starting materials:
3-(5-Chlorothiophen-2-yl)-5-(trifluoromethyl)pyrazolo[1,5-a]pyridin-2-amine
3-(6-Chloropyridin-3-yl)-5-(trifluoromethyl)pyrazolo[1,5-a]pyridin-2-amine

### Method 4: Exemplary Procedures for Bromination and Subsequent Cross Coupling of 3-unsubstituted pyrazolo[1,5-a]pyridin-2-amines (Scheme 3)

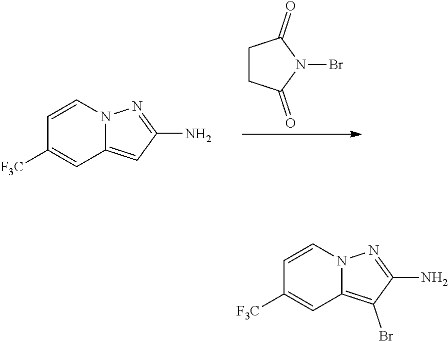 [SMILES: Nc1cc2cc(C(F)(F)F)ccn2n1 ; O=C1CCC(=O)N1Br ; Nc1nn2ccc(C(F)(F)F)cc2c1Br]

Step A. Preparation of 3-bromo-5-(trifluoromethyl)pyrazolo[1,5-a]pyridin-2-amine. The procedure of Method 3, Step A was followed, with the substitution of N-bromosuccinimide for N-iodosuccinimide, to produce the title compound. NMR (CDCl₃) δ 8.29 (d, J=7.1 Hz, 1H), 7.68 (s, 1H), 7.53-7.43 (m, 1H), 7.33-7.28 (m, 1H), 7.25-7.20 (m, 1H), 7.07-7.00 (m, 1H), 6.76 (dd, J=1.9, 7.2 Hz, 1H), 4.25 (s, 1H). MS (ESI) m/z 328.0 (MH⁺)

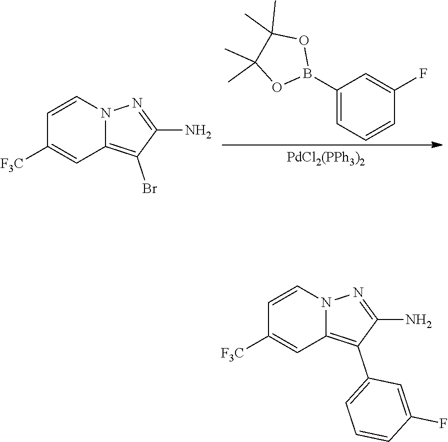 [SMILES: Nc1nn2ccc(C(F)(F)F)cc2c1Br ; CC1(C)OB(c2cccc(F)c2)OC1(C)C ; Nc1nn2ccc(C(F)(F)F)cc2c1-c1cccc(F)c1]

Step B. Preparation of 3-(3-fluorophenyl)-5-(trifluoromethyl)-pyrazolo[1,5-a]pyridin-2-amine The procedure of Method 3, Step B was followed, with the substitution of 4,4,5,5-tetramethyl-2-(3-fluorophenyl)-1,3,2-dioxaborolane for 4,4,5,5-tetramethyl-2-(p-tolyl)-1,3,2-dioxaborolane, to produce the title compound. NMR (CDCl₃) δ 8.29 (d, J=7.1 Hz, 1H) 7.68 (s, 1H), 7.53-7.43 (m, 1H), 7.33-7.28 (m, 1H), 7.25-7.20 (m, 1H), 7.07-7.00 (m, 1H) 6.76 (dd, J=1.9, 7.2 Hz, 1H), 4.25 (s, 2H). MS (ESI) m/z 296.0 (MH⁺).

The following pyrazolo[1,5-a]pyridin-2-amines were prepared using the general procedure described in Section 1, Method 4, Step B, with appropriate starting materials:
3-Phenyl-5-(trifluoromethyl)-pyrazolo[1,5-a]pyridin-2-amine
3-(4-Chlorophenyl)-5-(trifluoromethyl)pyrazolo[1,5-a]pyridin-2-amine

### Section 2. Representative Procedures for the Preparation of pyrazolo[1,5-a]pyridin-2,3-yl amides (Compounds 1.6, Scheme 1)

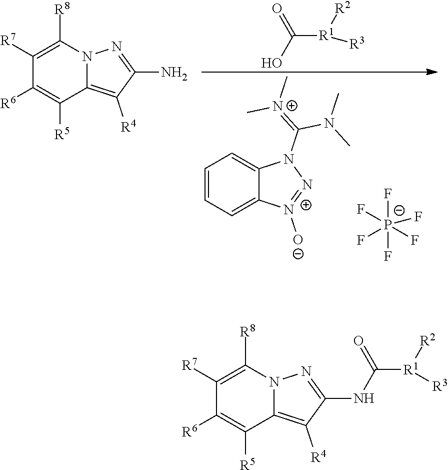

### Method 5: General Procedure for Amide Formation Using HATU (1-((dimethylamino)(dimethyliminio)methyl)-1H-benzo[d][1,2,3]triazole 3-oxide hexafluorophosphate(V)) and N,N-diisopropylethylamine

To a solution of appropriate carboxylic acid 1.5 (1.2-2.0 molar equivalents) in DMF, N-methylpyrrolidinone, or THF (0.1-1 M) was added HATU (1.8 molar equivalents) and N,N-diisopropylethylamine (2.0 molar equivalents). The appropriate pyrazolo[1,5-a]pyridin-2-amine (1.0 molar equivalents) was added. The mixture was heated to 50-60° C. for 18-48 h. The mixture was then diluted with EtOAc and washed sequentially with saturated aqueous NaHCO₃ and water (4 times). The organic layer was dried over sodium sulfate and concentrated. Purification by column chromatography (0-100% EtOAc/hexanes or 0-10% MeOH/CH₂Cl₂) provided the amide compounds.

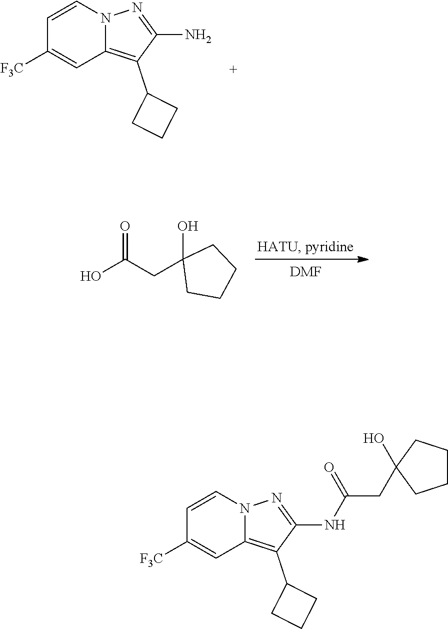 [SMILES: Nc1nn2ccc(C(F)(F)F)cc2c1C1CCC1 ; O=C(O)CC1(O)CCCC1 ; O=C(CC1(O)CCCC1)Nc1nn2ccc(C(F)(F)F)cc2c1C1CCC1]

### Method 6: Exemplary Procedure for Amide Formation Using HATU (1-((dimethylamino)(dimethyliminio)methyl)-1H-benzo[d][1,2,3]triazole 3-oxide hexafluorophosphate(V)) and pyridine. Preparation of N-(3-cyclobutyl-5-(trifluoromethyl)pyrazolo[1,5-a]pyridin-2-yl)-2-(1-hydroxycyclopentyl)acetamide

To a solution of 2-(1-hydroxycyclopentyl) acetic acid (27 mg, 0.19 mmol) in DMF (0.3 mL) was added HATU (119 mg, 0.31 mmol) and pyridine (25 μL and 0.31 mmol) at room temperature. After 5 minutes, 3-cyclobutyl-5-(trifluoromethyl)pyrazolo[1,5-a]pyridin-2-amine (40 mg, 0.16 mmol) was added. The mixture was stirred at room temperature for 18 hours. The crude reaction mixture was directly purified by reverse phase chromatography (10-100% acetonitrile/water). Additional purification by column chromatography (0-100% EtOAc/DCM) provided N-(3-cyclobutyl-5-(trifluoromethyl)pyrazolo[1,5-a]pyridin-2-yl)-2-(1-hydroxycyclopentyl)acetamide (13 mg, 0.034 mmol). (ESI) m/z 382.4 (M+H).

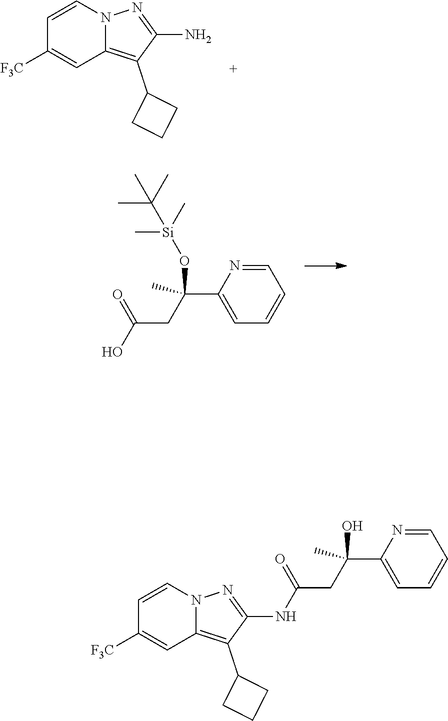

### Method 7: Exemplary Procedure for Amide Formation Using HATU (1-((dimethylamino)(dimethyliminio)methyl)-1H-benzo[d][1,2,3]triazole 3-oxide hexafluorophosphate(V)) and pyridine, Using silyl-protected hydroxy acid, Followed by Deprotection of Silyl Group. Preparation of (R)—N-(3-cyclobutyl-5-(trifluoromethyl)pyrazolo[1,5-a]pyridin-2-yl)-3-hydroxy-3-(pyridin-2-yl) butanamide

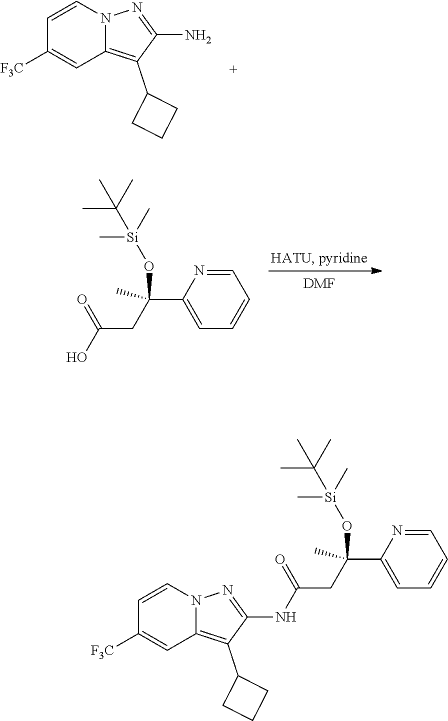 [SMILES: Nc1nn2ccc(C(F)(F)F)cc2c1C1CCC1 ; CC(C)(C)[Si](C)(C)O[C@](C)(CC(=O)O)c1ccccn1 ; CC(C)(C)[Si](C)(C)O[C@](C)(CC(=O)Nc1nn2ccc(C(F)(F)F)cc2c1C1CCC1)c1ccccn1]

Step A. Preparation of (R)-3-((tert-butyldimethylsilyl)oxy)-N-(3-cyclobutyl-5-(trifluoromethyl)pyrazolo[1,5-a]pyridin-2-yl)-3-(pyridin-2-yl) butanamide. To (R)-3-((tert-butyldimethylsilyl)oxy)-3-(pyridin-2-yl) butanoic acid (35 mg, 0.12 mmol) in DMF (0.3 mL) was added pyridine (19 μL, 0.24 mmol) and HATU (67 mg, 0.18 mmol) and the mixture was stirred at room temperature for 10 min. 3-Cyclobutyl-5-(trifluoromethyl)pyrazolo[1,5-a]pyridin-2-amine (30 mg, 0.12 mmol) was added and the mixture was stirred at 50° C. overnight. The solution was cooled to room temperature, then diluted with EtOAc and washed sequentially with H₂O and brine, dried (Na₂SO₄), filtered and concentrated to provide the crude amide, which was taken into Step B without further purification.

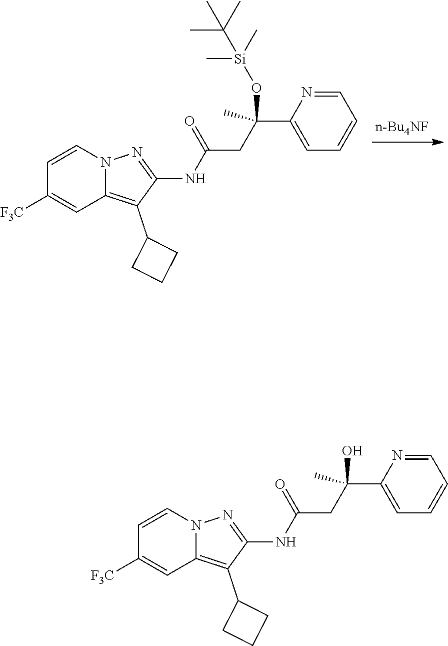 [SMILES: CC(C)(C)[Si](C)(C)O[C@](C)(CC(=O)Nc1nn2ccc(C(F)(F)F)cc2c1C1CCC1)c1ccccn1 ; C[C@@](O)(CC(=O)Nc1nn2ccc(C(F)(F)F)cc2c1C1CCC1)c1ccccn1]

Step B. Preparation of (R)—N-(3-cyclobutyl-5-(trifluoromethyl)pyrazolo[1,5-a]pyridin-2-yl)-3-hydroxy-3-(pyridin-2-yl) butanamide. The crude material from Step A was dissolved in THF (0.35 mL) and 1.0 M tetra(n-butyl)ammonium fluoride in THF (1 mL) was added. The resulting mixture was stirred at room temperature for 2 h. The solvent was removed in vacuo, and the crude material was purified by column chromatography (gradient elution with 0-100% EtOAc/hexanes), followed by a second purification via HPLC (10-100% CH₃CN/H₂O) to give the title compound (29.4 mg, 59%). NMR (CDCl₃) δ 8.86 (s, 1H), 8.54 (d, J=4.4 Hz, 1H), 8.38 (d, J=8.4 Hz, 1H) 7.82-7.71 (m, 2H), 7.48 (d, J=8.0 Hz, 1H), 6.77 (d, J=8.0 Hz, 1H), 6.14 (s, 1H), 3.58-3.44 (m, 1H), 3.02 (dd, J=14.8, 28.4 Hz, 2H), 2.40-2.17 (m, 4H), 2.12-1.97 (m, 1H), 1.97-1.83 (m, 1H). MS (ESI) m/z 419.2 (MH⁺).

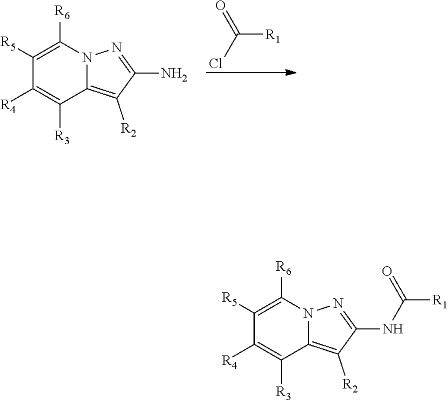

### Method 8: General Procedure for Amide Formation Using Acyl Chlorides

To a solution of appropriate pyrazolo[1,5-a]pyridin-2-amine (1 equiv) in THF (0.1 M) was added triethylamine (2 equiv.) and acyl chloride (1.2 equiv.) at ambient temperature. The reaction mixture was stirred for 4 h. The mixture was partitioned between EtOAc and water. The organic layer was dried over sodium sulfate and concentrated. The residue was purified by column chromatography (0-100% EtOAc/hexanes or 0-10% MeOH/CH₂Cl₂) to provide the amide compounds.

### Section 3. Exemplary Syntheses for Carboxylic Acids (1.7 in Scheme 1), Useful for Coupling Reactions with pyrazolo[1,5-a]pyridin-2,3-yl amines

### Method 9. Preparation of (S)-3-hydroxy-3-phenylbutanoic acid

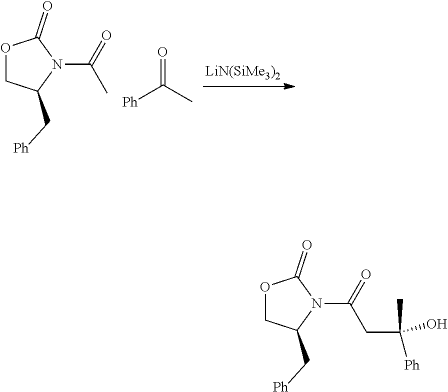

Step A. Preparation of (S)-4-benzyl-3-((S)-3-hydroxy-3-phenylbutanoyl) oxazolidin-2-one. A solution of LiN(TMS)₂ (1.0 M in THF, 9.2 mL, 9.2 mmol) was added dropwise to a −78° C. mixture of (S)-3-acetyl-4-benzyloxazolidin-2-one (2.0 g, 9.2 mmol) in THF (8 mL). The mixture was stirred for 2 hours at −78° C. and then acetophenone (0.5 g, 4.2 mmol) was added over 10 minutes. The −78° C. mixture was stirred for one hour and then quenched via the addition of 1.0 N HCl. The mixture was warmed to room temperature and extracted three times with dichloromethane. The combined organics were washed with brine, dried over anhydrous Na₂SO₄ and concentrated. The residue was purified by silica gel chromatography (0-50% EtOAc/hexanes) to provide a product that was further purified by recrystallization from hot MTBE to provide the desired product (590 mg) as a single diastereomer by NMR. Stereochemical assignment derived from Theurer, et al, Tetrahedron, 2010, 66, 3814.

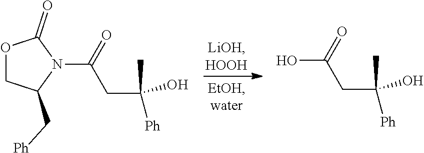 [SMILES: C[C@](O)(CC(=O)N1C(=O)OC[C@@H]1Cc1ccccc1)c1ccccc1 ; C[C@](O)(CC(=O)O)c1ccccc1]

Step B. Preparation of (S)-3-hydroxy-3-phenylbutanoic acid. To a solution of (S)-4-benzyl-3-((S)-3-hydroxy-3-phenylbutanoyl) oxazolidin-2-one (590 mg, 1.7 mmol) in 1:1 THF:H₂O (9 mL) was added 30% aqueous H₂O₂ (788 μL, 7.0 mmol) and LiOH (167 mg, 7.0 mmol). The mixture was vigorously stirred for 2 hours and then the reaction was partitioned between water (50 mL) and EtOAc (25 mL). The aqueous layer was isolated and the pH was adjusted to pH=2 with 1.0 N HCl. The aqueous mixture was extracted three times with EtOAc. The combined organics were washed with brine, dried (Na₂SO₄) and concentrated to afford the crude desired product which was used as is in the following coupling reactions. (ESI) m/z 179.2 (M−H).

### Method 10. Preparation of (R)-3-((tert-butyldimethylsilyl)oxy)-3-(pyridin-2-yl) butanoic acid

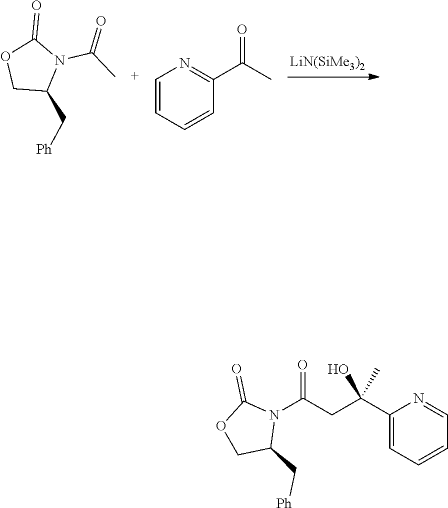

Step A: Preparation of (S)-4-benzyl-3-((R)-3-hydroxy-3-(pyridin-2-yl) butanoyl) oxazolidin-2-one. Lithium bis(trimethylsilyl)amide (1.0 M tetrahydrofuran, 6.9 mL, 6.9 mmol) was added over 15 minutes to a −78° C. suspension of (S)-3-acetyl-4-benzyloxazolidin-2-one (1.52 g, 6.9 mmol) in tetrahydrofuran (12 mL). The mixture was stirred at −78° C. for two hours. A solution of 2-acetylpyridine (800 mg, 6.6 mmol) in tetrahydrofuran (4 mL) was added over 35 minutes. The mixture was stirred at −78° C. for one hour and then quenched via the addition of aqueous 0.5 M HCl. The mixture was warmed to room temperature and then extracted with CH₂Cl₂. The layers were separated and the aqueous phase was extracted twice more with CH₂Cl₂. The combined organics were dried over anhydrous Na₂SO₄ and concentrated. The residue was purified by silica gel chromatography (0-30% EtOAc/hexanes) to provide the desired compound in a partially purified fashion (1.9 g) that was used as is.

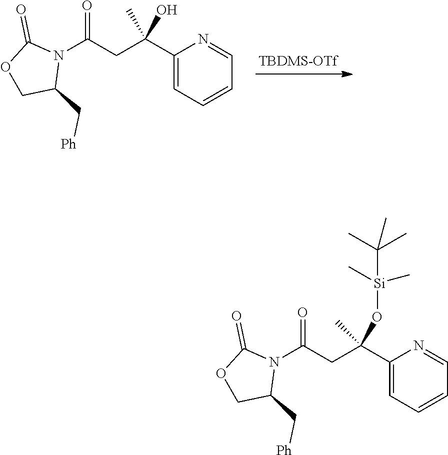 [SMILES: C[C@@](O)(CC(=O)N1C(=O)OC[C@@H]1Cc1ccccc1)c1ccccn1 ; CC(C)(C)[Si](C)(C)O[C@](C)(CC(=O)N1C(=O)OC[C@@H]1Cc1ccccc1)c1ccccn1]

Step B: Preparation of (S)-4-benzyl-3-((R)-3-((tert-butyldimethylsilyl)oxy)-3-(pyridin-2-yl) butanoyl) oxazolidin-2-one. tert-Butyldimethylsilyl trifluoromethanesulfonate (1.4 mL, 6.1 mmol) was added dropwise to a room temperature solution of the residue prepared as described in Step A and Et₃N (1.2 mL, 8.4 mmol) in CH₂Cl₂ (33 mL). The mixture was stirred at room temperature overnight and then partitioned between EtOAc and saturated aqueous NaHCO₃. The phases were separated and the organics were washed with saturated aqueous NaCl. The two aqueous phases were then sequentially extracted twice with EtOAc. The combined organics were dried over anhydrous Na₂SO₄ and concentrated. The residue was purified twice by silica gel chromatography (0-15% EtOAc/hexanes to provide the expected product (1.55 g, 61%). NMR (CDCl₃) δ 8.47 (d, J=5.4 Hz, 1H), 7.75-7.68 (m, 2H), 7.33-7.21 (m, 3H), 7.17-7.10 (m, 3H), 4.62-4.53 (m, 1H), 4.07 (d, J=4.4 Hz, 2H), 3.87 (d, J=15.6 Hz, 1H), 3.64 (d, J=15.6 Hz, 1H), 3.18-3.11 (m, 1H), 2.69-2.59 (m, 1H), 0.94 (s, 9H), 0.14 (s, 3H), 0.09 (s, 3H).

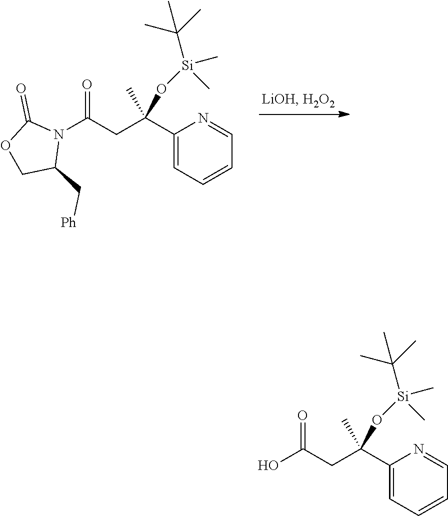 [SMILES: CC(C)(C)[Si](C)(C)O[C@](C)(CC(=O)N1C(=O)OC[C@@H]1Cc1ccccc1)c1ccccn1 ; CC(C)(C)[Si](C)(C)O[C@](C)(CC(=O)O)c1ccccn1]

Step C: Preparation of (R)-3-((tert-butyldimethylsilyl)oxy)-3-(pyridin-2-yl) butanoic acid. Lithium hydroxide (0.8 M in H₂O, 21.3 ml, 17.0 mmol) and 30% aqueous hydrogen peroxide (1.74 mL, 17.0 mmol) were added to a 0° C. mixture of (S)-4-benzyl-3-((R)-3-((tert-butyldimethylsilyl)oxy)-3-phenylbutanoyl) oxazolidin-2-one from Step B (1.55 g, 3.4 mmol) in tetrahydrofuran (20 mL). The mixture was stirred at 0° C. to room temperature over 80 minutes. The mixture was adjusted to pH 2 via the addition of 1 M aqueous HCl and then extracted with EtOAc. The organics were combined, washed with saturated aqueous NaCl, dried over anhydrous Na₂SO₄ and concentrated. The residue was purified by silica gel chromatography (0-30% EtOAc/hexanes) to provide the expected product (0.70 g, 69%). NMR (CDCl₃) δ 16.00 (s, 1H), 8.47 (d, J=5.2 Hz, 1H), 7.89 (t, J=7.4, 14.8 Hz, 1H), 7.75 (d, J=8.8 Hz, 1H), 7.37 (t, J=6.6, 13.2 Hz, 1H), 3.25 (d, J=14.4 Hz, 1H), 2.99 (d, J=13.2 Hz, 1H), 1.69 (s, 3H), 0.93 (s, 9H), 0.145 (s, 3H), 0.135 (s, 3H).

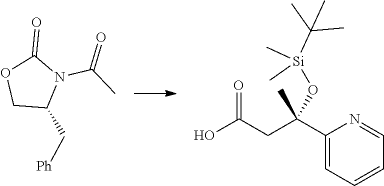 [SMILES: CC(=O)N1C(=O)OC[C@H]1Cc1ccccc1 ; CC(C)(C)[Si](C)(C)O[C@@](C)(CC(=O)O)c1ccccn1]

### Method 11. Preparation of (S)-3-((tert-butyldimethylsilyl)oxy)-3-(pyridin-2-yl) butanoic acid

The title compound was prepared in the same manner as Step A above, but using (R)-3-acetyl-4-benzyloxazolidin-2-one in place of (S)-3-acetyl-4-benzyloxazolidin-2-one.

### Method 12. Exemplary Synthesis of 3,3-dialkyl-3-hydroxypropanoic acids. Preparation of 2-(1-hydroxycyclopentyl) acetic acid

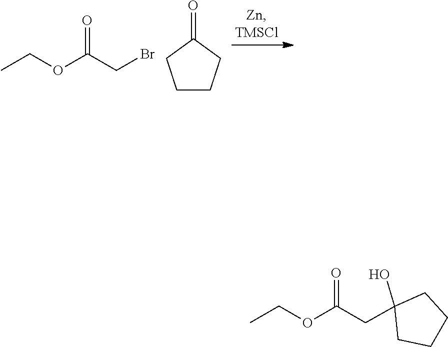

Step A. Preparation of ethyl 2-(1-hydroxycyclopentyl)acetate. Chlorotrimethylsilane (181 μL, 1.4 mmol) was added to a suspension of zinc powder (1.2 g, 19 mmol) in Et₂O (30 mL). The mixture was stirred at room temperature for 15 minutes and then refluxed for 15 minutes. The heat source was removed and ethyl bromoacetate (1.8 mL, 14 mmol) was added dropwise to the warm mixture. The mixture was then refluxed for one hour and then stirred at room temperature for one hour. Cyclopentanone (1.0 g, 12 mmol) was then added dropwise. The resulting mixture was stirred for one hour and then poured into ice cold concentrated aqueous ammonia (80 mL). The layers were separated and the aqueous phase was extracted with Et₂O (3×40 mL). The combined organics were dried (K₂CO₃) and concentrated to yield 1.7 g of a colorless oil. This material was used as is in the next step.

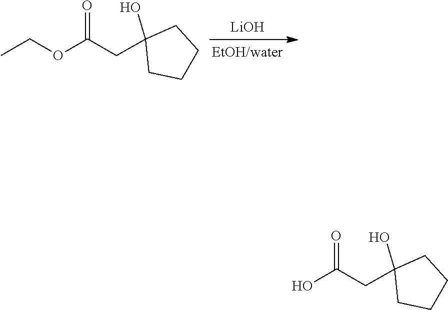

Step B. Preparation of 2-(1-hydroxycyclopentyl) acetic acid. Lithium hydroxide (1.4 g, 58 mmol) was added to a room temperature solution of the crude ester prepared as described in the previous step (1.0 g, 5.8 mmol) in 1:1 EtOH:water (29 mL). After 2 hours, the reaction was partitioned between water (100 mL) and MTBE (100 mL). The aqueous layer was isolated and the pH was adjusted to pH=2 with 1.0 N HCl. The aqueous mixture was extracted three times with EtOAc. The combined organics were dried (Na₂SO₄) and concentrated to afford the desired product (500 mg, 60%). (ESI) m/z 143.2 (M−H).

The following carboxylic acids were prepared using the general procedure described in Method 12 with appropriate starting materials.
2-(1-Hydroxycyclobutyl) acetic acid
3-Cyclopropyl-3-hydroxybutanoic acid
3-Cyclobutyl-3-hydroxybutanoic acid
3-Cyclopentyl-3-hydroxybutanoic acid

### General Analytical Methods

LCMS was conducted on an Agilent 1100 MSD instrument equipped with an Ascentis Express C18, 10 cm×4.6 mm×2.7 mm column, using the following methods:

### HPLC Method A

Solvent A: 0.1% formic acid in water
Solvent B: Acetonitrile
Flow rate: 1.4 mL/min

### Method:

0-6.0 min gradient from B=10% to B=95%
6.0-8.0 min, hold B=95%
8.0-8.2 min, gradient from B=95% to B=10%
8.2-10.0 min, hold B=10%

### HPLC Method B:

Solvent A: 0.1% formic acid in water
Solvent B: Acetonitrile
Flow rate: 1.4 mL/min

### Method:

0-3.0 min gradient from B=10% to B=95%
3.0-4.0 min, hold B=95%
4.0-4.2 min, gradient from B=95% to B=10%
4.2-6.0 min, hold B=10%

### Biological Assay Methods

### Kv7.2/7.3 Activation Assay

The ability of compounds to potentiate K-currents in Kv7.2/7.3 containing HEK cells was assessed using planar patch-clamp on the QPatch automated screening platform.

Cell Line: The hKv7.2/7.3 cell line was obtained from Chantest (Cleveland, OH 44128) cat. #CT6147. These HEK cells will express the Kv7.2/7.3 ion channels when induced.

Cell Culture: Cells were maintained in a media containing DMEM/F12; 50/50 (GIBCO cat. #11330), 10% Fetal Bovine Serum (FBS) (GIBCO cat. #26140), 100 units/mL Penicillin-Streptomycin (GIBCO cat. #15140), 0.005 mg/mL Blasticidin (INVIVOGEN cat. #ant-bl-1), 0.5 mg/mL Geneticin (GIBCO cat. #10131), 0.1 mg/mL Zeocin (GIBCO cat. #R25001). Cells used in the electrophysiology assay were maintained in a media without Blasticidin, Geneticin and Zeocin for 2 days and channel expression was induced by adding tetracycline (BIOLINE cat. #BIO-87030) at a final concentration of 1 mg/mL. Cells were grown in T-175 flask to ˜75% confluency. Currents were recorded 24 hours after channel induction.

Compound Plates: Test compounds were prepared by performing serial dilutions on a Biomek NX^{P} (BECKMAN COULTER). Final dilutions were made in external recording solution with a final DMSO concentration of 0.1% DMSO. For single concentration screens each plate had 10 μM retigabine as a positive control and 0.1% DMSO as a negative control.

Electrophysiology: On the day of the experiment cells were washed with Hank's Balanced Salt Solution (HBBS) (GIBCO cat. #14175) and harvested with Tryple (GIBCO cat. #12604). Cells were then centrifuged at 2000 rpm for 5 minutes and resuspended in CHO—S-SFM (GIBCO cat. #12052) at −3×10⁶ cells/mL. Cells were stirred for 30 minutes before experiments were started. External recording solution contained (in mM): NaCl (145), KCl (4), CaCl₂ (2), MgCl₂ (1), HEPES (10) and Glucose (10); pH was adjusted to 7.4 with NaOH and the osmolarity was adjusted to 300-305 mOsM with sucrose if necessary. Internal solution contained (in mM): KCl (125), KF (10), EGTA (5), Na₂ATP (5), MgCl₂ (3.2), HEPES (5); pH was adjusted to 7.2 with KOH and the osmolarity was adjusted to 298-302 mOsM with sucrose.

Potassium channel activity was measured on the QPatch HTX (Sophion Bioscience) using QPlates with 48-wells/plate. Each cell was taken as an independent experiment and only one compound was tested per well. Potassium channel activity was elicited by holding at −80 mV and stepping to −30 mV for 2 s followed by a 100 ms pulse to −120 mV.

Single concentration screen: Baseline conditions were obtained by recording 5 sweeps in the external solution only, this was repeated for three applications of the external solution. The effect of test compounds on elicited current was then assessed by recording 5 sweeps in the presence of a 3 μM compound solution. The steady-state current at the end of the 2 s pulse to −30 mV was measured to determine the fold increase from baseline.

Data of the Kv7.2/7.3 Activation Assay is summarized in Table 2

TABLE 2
Kv7.2/7.3 QPatch Single Concentration Screen Results

| | QPatch Kv7.2/7.3 |
|---|---|
| Example | Activity* |
| 1 | + |
| 2 | + |
| 3 | + |
| 4 | +/− |
| 5 | +/− |
| 6 | + |
| 7 | + |
| 8 | + |
| 9 | − |
| 10 | + |
| 11 | +/− |
| 12 | + |
| 13 | + |
| 14 | + |
| 16 | +/− |
| 17 | + |
| 18 | + |
| 19 | + |
| 20 | + |
| 21 | + |
| 22 | + |
| 23 | + |
| 24 | ++ |
| 25 | ++ |
| 26 | + |
| 27 | + |
| 28 | +/− |
| 29 | ++ |
| 30 | + |
| 31 | + |
| 32 | + |
| 33 | + |
| 34 | − |
| 35 | + |
| 36 | + |
| 37 | ++ |
| 38 | ++ |
| 39 | + |
| 40 | + |
| 41 | ++ |
| 42 | + |
| 43 | + |
| 44 | + |
| 45 | +/− |
| 46 | +/− |
| 47 | + |
| 48 | + |
| 49 | + |
| 50 | + |
| 51 | +/− |
| 52 | − |
| 53 | + |
| 54 | − |
| 55 | + |
| 56 | + |
| 57 | ++ |
| 58 | + |
| 59 | + |
| 60 | ++ |
| 61 | +/− |
| 62 | + |
| 63 | + |
| 64 | +++ |
| 65 | ++ |
| 66 | − |
| 67 | − |
| 68 | + |
| 69 | + |
| 70 | + |
| 71 | + |
| 72 | +/− |
| 73 | − |
| 74 | ++ |
| 75 | − |
| 76 | + |
| 77 | + |
| 78 | + |
| 79 | ++ |
| 80 | − |
| 81 | ++ |
| 82 | +++ |
| 83 | + |
| 84 | +/− |
| 85 | − |
| 86 | +/− |
| 87 | + |
| 88 | ND |
| 89 | ND |
| 90 | ND |
| 91 | ND |
| 92 | ND |
| 93 | ND |
| 94 | ND |
| 95 | + |
| 96 | ND |
| 97 | ND |
| 98 | ND |

*Increase in current from Kv7.2/Kv7.3 co-expressing HEK cells, measured at compound concentration of 3 μM, indicated as <1.2-fold (−), 1.2-1.99-fold (+/−), 2.0-3.99 (+), 4.0-5.99 (++), or >6-fold (+++) increase over baseline. ND stands for Not Determined

The Thallium Flux Assay is used as a surrogate indicator of potassium channel activity.

The experimental protocol was adapted from the FluxOR™ II Green Potassium Ion Channel Assay User Guide (Pub. No. MAN0016084, Invitrogen). Conditions were optimized for the Kv7.2/7.3 cell line.

Cell Line: The hKv7.2/7.3 cell line was obtained from Chantest (Cleveland, OH 44128) cat. #CT6147.

Cell Culture: Kv7.2/7.3 cells were maintained in a media containing DMEM/F12; 50/50 (GIBCO cat. #11330), 10% Fetal Bovine Serum (FBS) (GIBCO cat. #26140), 100 units/mL Penicillin-Streptomycin (GIBCO cat. #15140), 0.005 mg/ml Blasticidin (SIGMA 15205), 0.5 mg/mL Geneticin (GIBCO cat. #10131), and 0.1 mg/mL Zeocin (GIBCO cat. #R25001). One day prior to experimentation, cells were plated in 96 well clear bottom plates (Corning cat. #353219) in a media without Blasticidin, Geneticin, or Zeocin. Channel expression was induced by adding tetracycline (Bioline cat. #BIO87030) at a final concentration of 10 ng/mL.

Compound Plates: The test compound is diluted in a mixture of 0.1% DMSO/extracellular solution with an eight-point concentration range from 0.014 μM to 30 μM. Serial dilutions were made on a Biomek NXP (BECKMAN COULTER).

Measurement and data analysis: A plate reader (Enspire, Perkin Elmer) is used to characterize the ion-channel modulating properties of novel compounds using an excitation wavelength of 475 nm and an emission wavelength of 530 nm. After a 15 sec baseline measurement, the stimulus buffer containing thallium and potassium is injected. A final endpoint measure is taken after 90 sec. Responses are normalized to positive control (retigabine, 30 μM max). Mean normalized responses at each concentration tested are fit to the standard Hill equation to generate an EC50 and maximal response.

Data of the Thallium Flux Assay is summarized in Table 3

TABLE 3
Kv7.2/7.3 Thallium Flux Assay Results

| | Thallium Flux Kv7.2/7.3 |
|---|---|
| Example | Activity^{a} |
| 3 | *** |
| 14 | *** |
| 57 | *** |
| 88 | NC |
| 89 | NC |
| 90 | NC |
| 91 | NC |
| 92 | NC |
| 93 | *** |
| 94 | *** |
| 95 | *** |
| 96 | NC |
| 97 | NC |
| 98 | *** |

^{a}Measured EC50 of Kv7.2/Kv7.3 activation using thallium flux assay as described in Biological Assay Methods section, described as a range from 3-10 μM (*), 1-3 μM (**), and <1 μM (***). ND stands for Not Determined. NC stands for Not Calculable

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A compound represented by a formula: 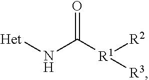
wherein Het is optionally substituted pyrazolo[1,5-a]pyridin-2-yl;
R¹ is C₁₋₆ linear or C₁₋₆ branched alkyl;
R² is H, OH, CF₃, or C₃₋₆-cycloalkyl-OH; and
R³ is H, CF₃, optionally substituted phenyl, optionally substituted pyridinyl, or optionally substituted C₃₋₆ cycloalkyl.

2. The compound of claim 1, wherein R² is H.

3. The compound of claim 1, wherein R² is CF₃.

4. The compound of claim 1, wherein R² is OH.

5. The compound of claim 1, wherein R² is cyclobutyl-OH or -cyclopentyl-OH.

6. The compound of claim 1, 2, 3, or 4, wherein R³ is H.

7. The compound of claim 1 or 3, wherein R³ is CF₃.

8. The compound of claim 1, 2, or 4, wherein R³ is optionally substituted phenyl.

9. The compound of claim 1, 2, or 4, wherein R³ is optionally substituted pyridinyl.

10. The compound of claim 1, 2, or 4, wherein R³ is optionally substituted cyclobutyl or optionally substituted cyclopentyl.

11. The compound of claim 1, wherein any substituents of Het, R¹ and R³ independently have a molecular weight of 15 Da to 200 Da and consist of 1 to 5 chemical elements, wherein the chemical elements are C, H, O, N, S, F, Cl, or Br.

12. The compound of claim 1, wherein any substituents of Het, R¹ and R³ are independently H, F, Cl, Br, I, C₁₋₆ alkyl, C₁₋₆ alkyl-OH, CF₃, CN, C₁₋₆ O-alkyl, or optionally substituted aryl.

13. The compound of claim 1, further represented by a formula: 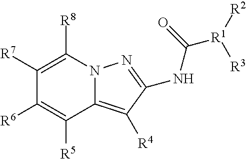
wherein R⁴ is H, F, Cl, Br, I, C₁₋₆ alkyl, C₁₋₆-alkyl-OH, CF₃, CN, C₁₋₆—O-alkyl, optionally substituted C₃₋₆ cycloalkyl, optionally substituted aryl, optionally substituted thiophenyl, or optionally substituted pyridinyl; and
R⁵, R⁶, R⁷, and R⁸ are each independently H, F, Cl, Br, I, C₁₋₆ alkyl, C₁₋₆-alkyl-OH, CF₃, CN, C₁₋₆—O-alkyl, optionally substituted C₃₋₆ cycloalkyl, or optionally substituted aryl.

14. The compound of claim 13, wherein R⁴ is cyclobutyl, optionally substituted phenyl, optionally substituted pyridinyl, or optionally substituted thiophenyl.

15. The compound of claim 1, which is:

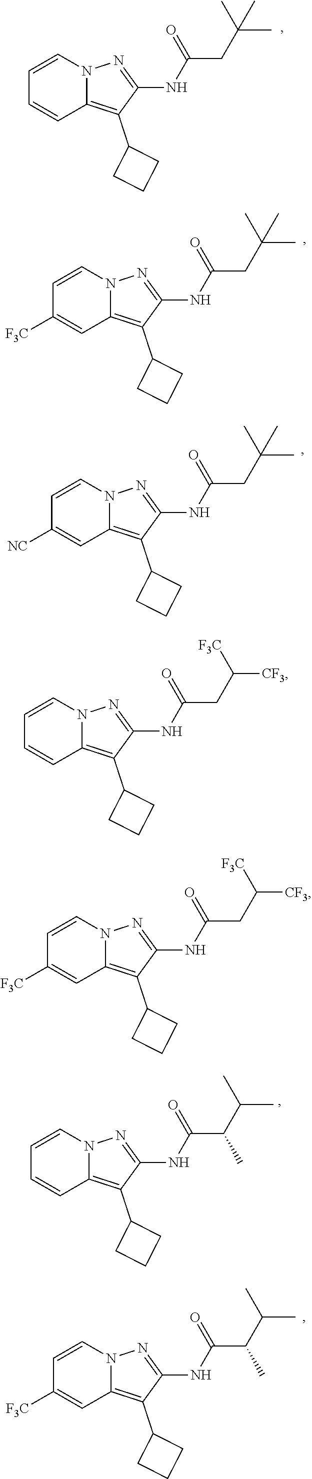

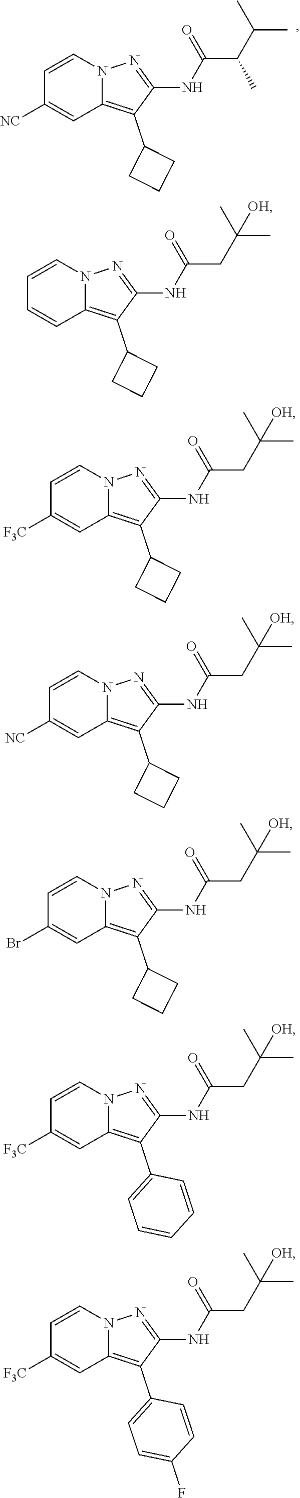

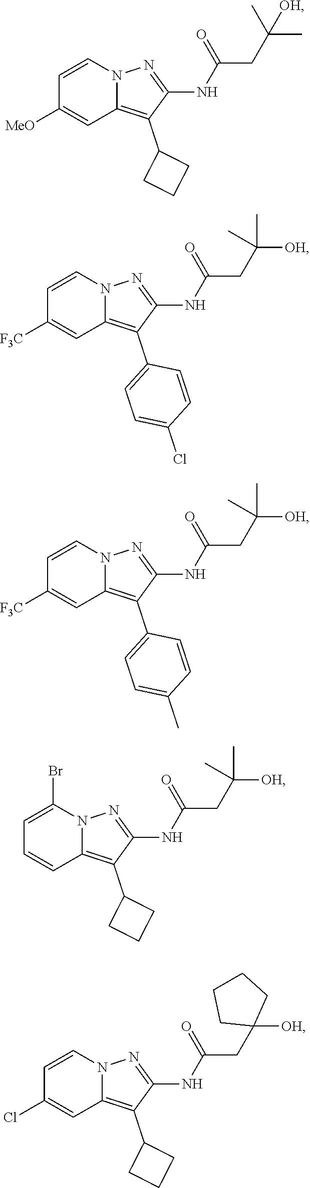

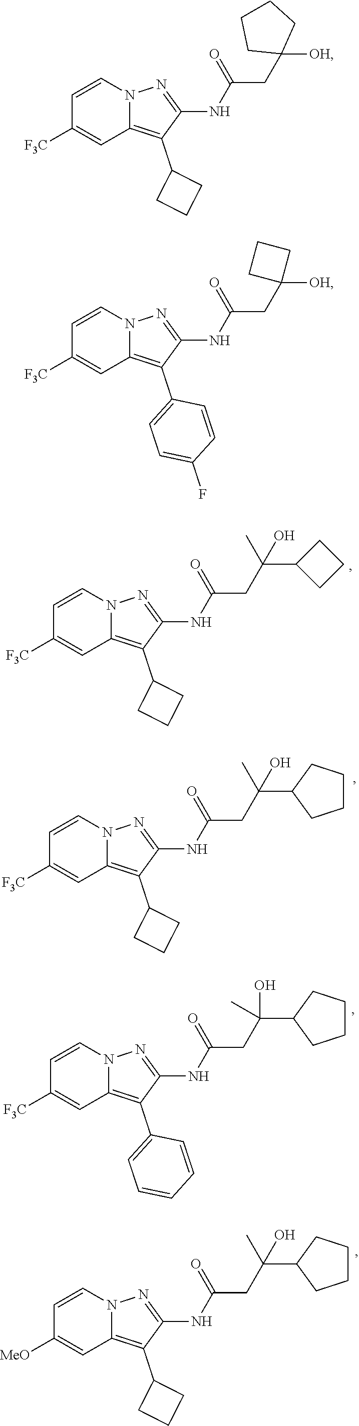 [SMILES: O=C(CC1(O)CCCC1)Nc1nn2ccc(C(F)(F)F)cc2c1C1CCC1 ; O=C(CC1(O)CCC1)Nc1nn2ccc(C(F)(F)F)cc2c1-c1ccc(F)cc1 ; CC(O)(CC(=O)Nc1nn2ccc(C(F)(F)F)cc2c1C1CCC1)C1CCC1 ; CC(O)(CC(=O)Nc1nn2ccc(C(F)(F)F)cc2c1C1CCC1)C1CCCC1 ; CC(O)(CC(=O)Nc1nn2ccc(C(F)(F)F)cc2c1-c1ccccc1)C1CCCC1 ; COc1ccn2nc(NC(=O)CC(C)(O)C3CCCC3)c(C3CCC3)c2c1]

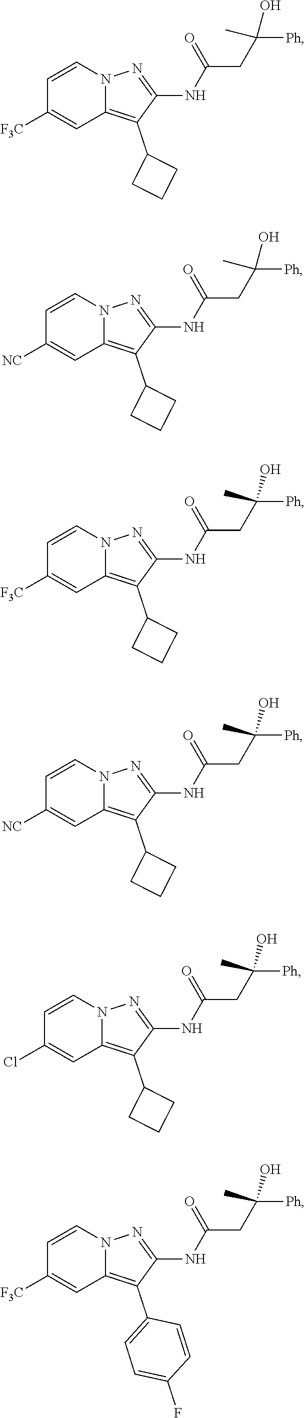 [SMILES: CC(O)(CC(=O)Nc1nn2ccc(C(F)(F)F)cc2c1C1CCC1)c1ccccc1 ; CC(O)(CC(=O)Nc1nn2ccc(C#N)cc2c1C1CCC1)c1ccccc1 ; C[C@](O)(CC(=O)Nc1nn2ccc(C(F)(F)F)cc2c1C1CCC1)c1ccccc1 ; C[C@](O)(CC(=O)Nc1nn2ccc(C#N)cc2c1C1CCC1)c1ccccc1 ; C[C@](O)(CC(=O)Nc1nn2ccc(Cl)cc2c1C1CCC1)c1ccccc1 ; C[C@](O)(CC(=O)Nc1nn2ccc(C(F)(F)F)cc2c1-c1ccc(F)cc1)c1ccccc1]

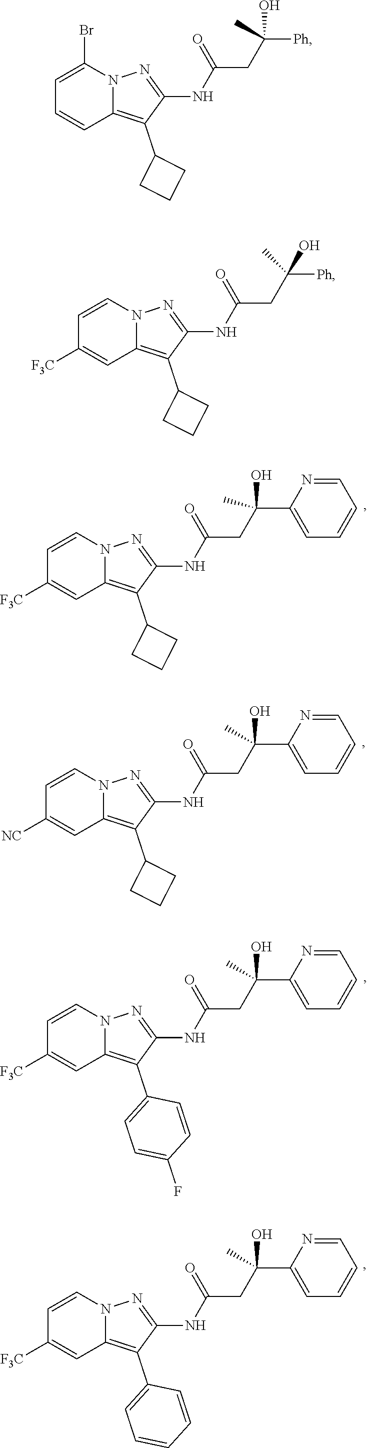 [SMILES: C[C@](O)(CC(=O)Nc1nn2c(Br)cccc2c1C1CCC1)c1ccccc1 ; C[C@@](O)(CC(=O)Nc1nn2ccc(C(F)(F)F)cc2c1C1CCC1)c1ccccc1 ; C[C@@](O)(CC(=O)Nc1nn2ccc(C(F)(F)F)cc2c1C1CCC1)c1ccccn1 ; C[C@@](O)(CC(=O)Nc1nn2ccc(C#N)cc2c1C1CCC1)c1ccccn1 ; C[C@@](O)(CC(=O)Nc1nn2ccc(C(F)(F)F)cc2c1-c1ccc(F)cc1)c1ccccn1 ; C[C@@](O)(CC(=O)Nc1nn2ccc(C(F)(F)F)cc2c1-c1ccccc1)c1ccccn1]

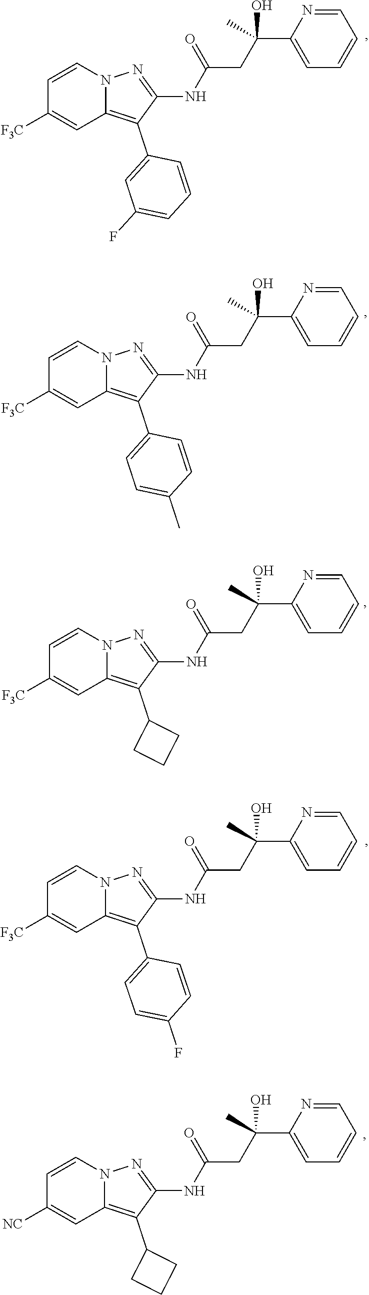 [SMILES: C[C@@](O)(CC(=O)Nc1nn2ccc(C(F)(F)F)cc2c1-c1cccc(F)c1)c1ccccn1 ; Cc1ccc(-c2c(NC(=O)C[C@@](C)(O)c3ccccn3)nn3ccc(C(F)(F)F)cc23)cc1 ; C[C@](O)(CC(=O)Nc1nn2ccc(C(F)(F)F)cc2c1C1CCC1)c1ccccn1 ; C[C@](O)(CC(=O)Nc1nn2ccc(C(F)(F)F)cc2c1-c1ccc(F)cc1)c1ccccn1 ; C[C@](O)(CC(=O)Nc1nn2ccc(C#N)cc2c1C1CCC1)c1ccccn1]

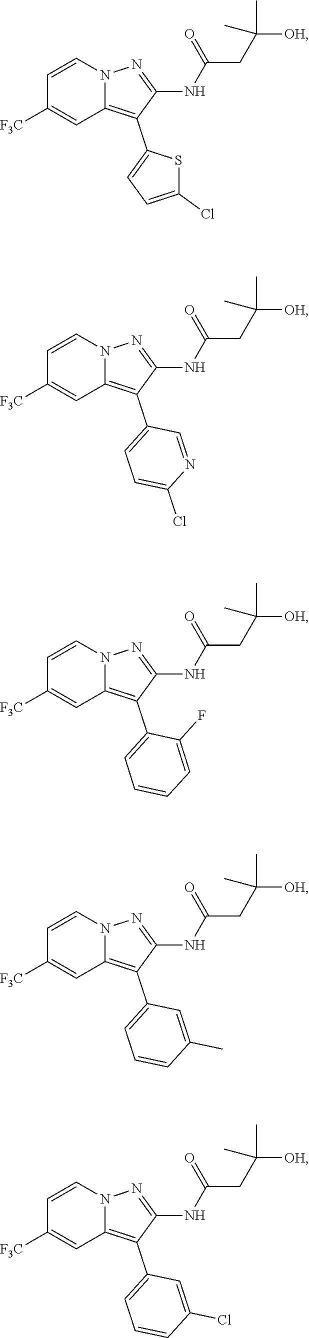 [SMILES: CC(C)(O)CC(=O)Nc1nn2ccc(C(F)(F)F)cc2c1-c1ccc(Cl)s1 ; CC(C)(O)CC(=O)Nc1nn2ccc(C(F)(F)F)cc2c1-c1ccc(Cl)nc1 ; CC(C)(O)CC(=O)Nc1nn2ccc(C(F)(F)F)cc2c1-c1ccccc1F ; Cc1cccc(-c2c(NC(=O)CC(C)(C)O)nn3ccc(C(F)(F)F)cc23)c1 ; CC(C)(O)CC(=O)Nc1nn2ccc(C(F)(F)F)cc2c1-c1cccc(Cl)c1]

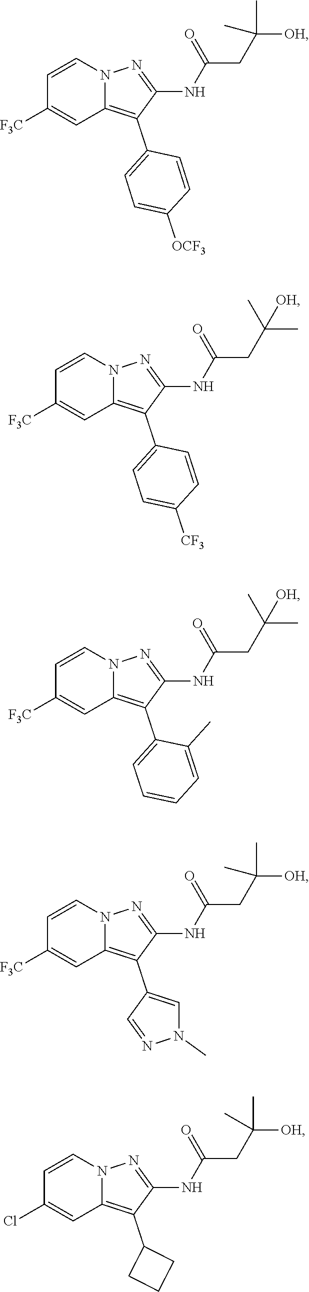 [SMILES: CC(C)(O)CC(=O)Nc1nn2ccc(C(F)(F)F)cc2c1-c1ccc(OC(F)(F)F)cc1 ; CC(C)(O)CC(=O)Nc1nn2ccc(C(F)(F)F)cc2c1-c1ccc(C(F)(F)F)cc1 ; Cc1ccccc1-c1c(NC(=O)CC(C)(C)O)nn2ccc(C(F)(F)F)cc12 ; Cn1cc(-c2c(NC(=O)CC(C)(C)O)nn3ccc(C(F)(F)F)cc23)cn1 ; CC(C)(O)CC(=O)Nc1nn2ccc(Cl)cc2c1C1CCC1]

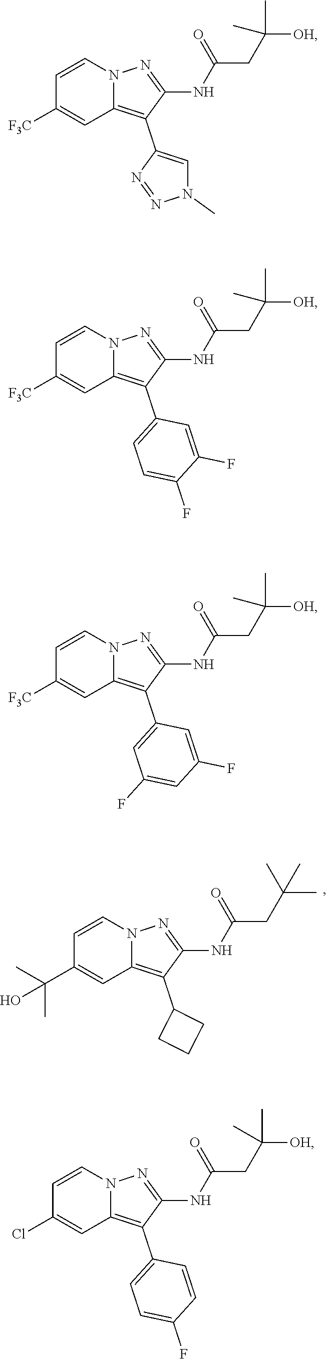 [SMILES: Cn1cc(-c2c(NC(=O)CC(C)(C)O)nn3ccc(C(F)(F)F)cc23)nn1 ; CC(C)(O)CC(=O)Nc1nn2ccc(C(F)(F)F)cc2c1-c1ccc(F)c(F)c1 ; CC(C)(O)CC(=O)Nc1nn2ccc(C(F)(F)F)cc2c1-c1cc(F)cc(F)c1 ; CC(C)(C)CC(=O)Nc1nn2ccc(C(C)(C)O)cc2c1C1CCC1 ; CC(C)(O)CC(=O)Nc1nn2ccc(Cl)cc2c1-c1ccc(F)cc1]

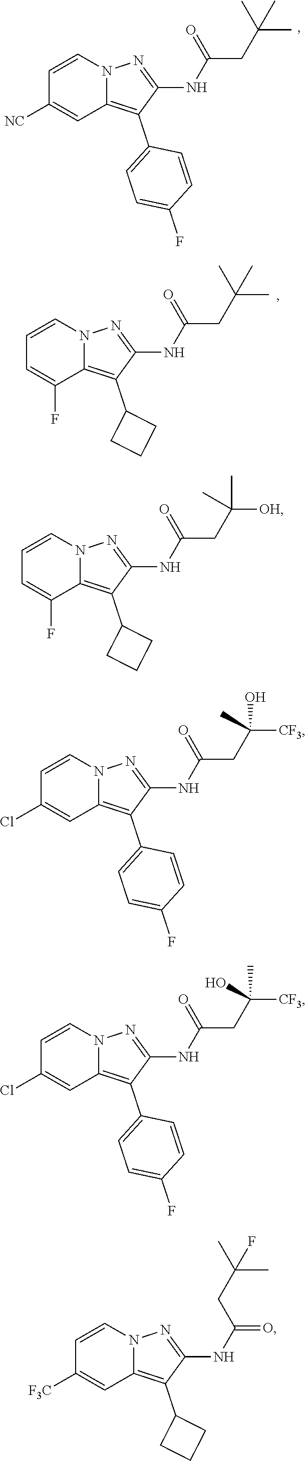

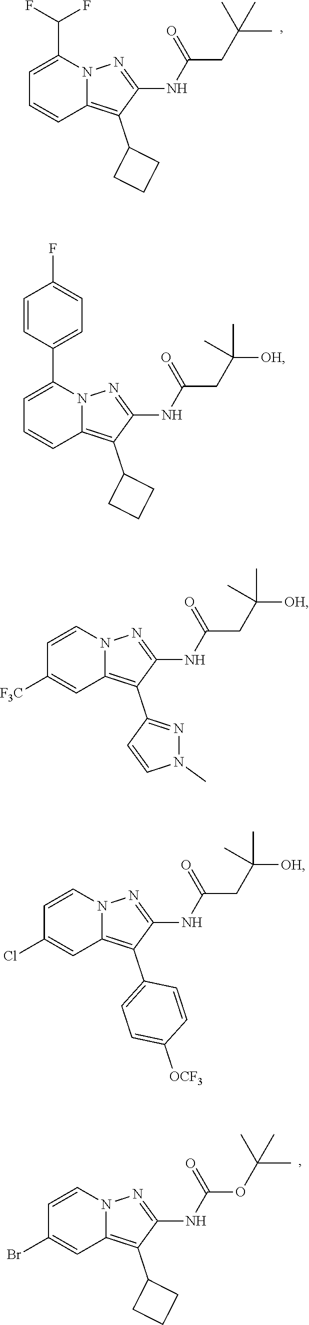 [SMILES: CC(C)(C)CC(=O)Nc1nn2c(C(F)F)cccc2c1C1CCC1 ; CC(C)(O)CC(=O)Nc1nn2c(-c3ccc(F)cc3)cccc2c1C1CCC1 ; Cn1ccc(-c2c(NC(=O)CC(C)(C)O)nn3ccc(C(F)(F)F)cc23)n1 ; CC(C)(O)CC(=O)Nc1nn2ccc(Cl)cc2c1-c1ccc(OC(F)(F)F)cc1 ; CC(C)(C)OC(=O)Nc1nn2ccc(Br)cc2c1C1CCC1]

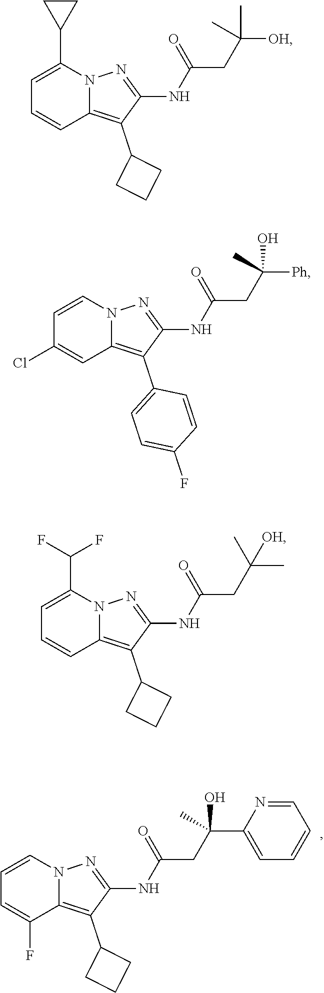 [SMILES: CC(C)(O)CC(=O)Nc1nn2c(C3CC3)cccc2c1C1CCC1 ; C[C@](O)(CC(=O)Nc1nn2ccc(Cl)cc2c1-c1ccc(F)cc1)c1ccccc1 ; CC(C)(O)CC(=O)Nc1nn2c(C(F)F)cccc2c1C1CCC1 ; C[C@@](O)(CC(=O)Nc1nn2cccc(F)c2c1C1CCC1)c1ccccn1]

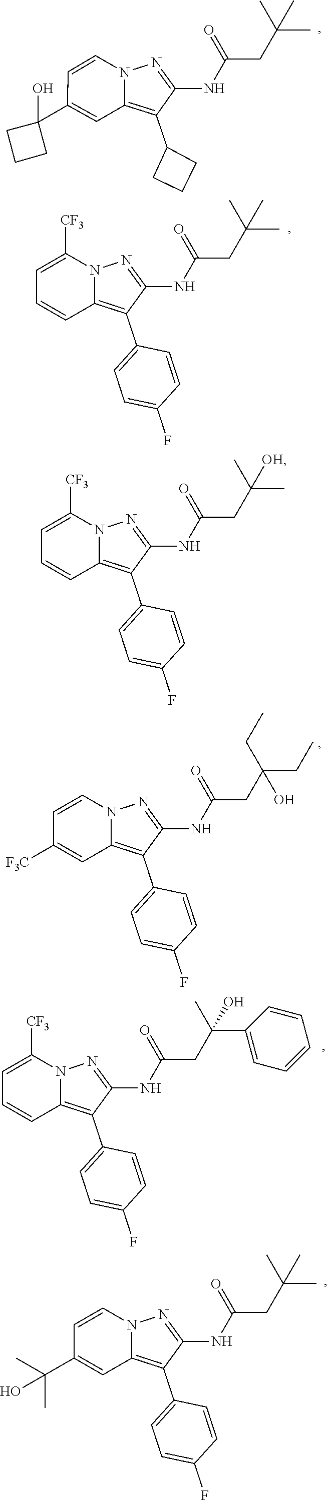

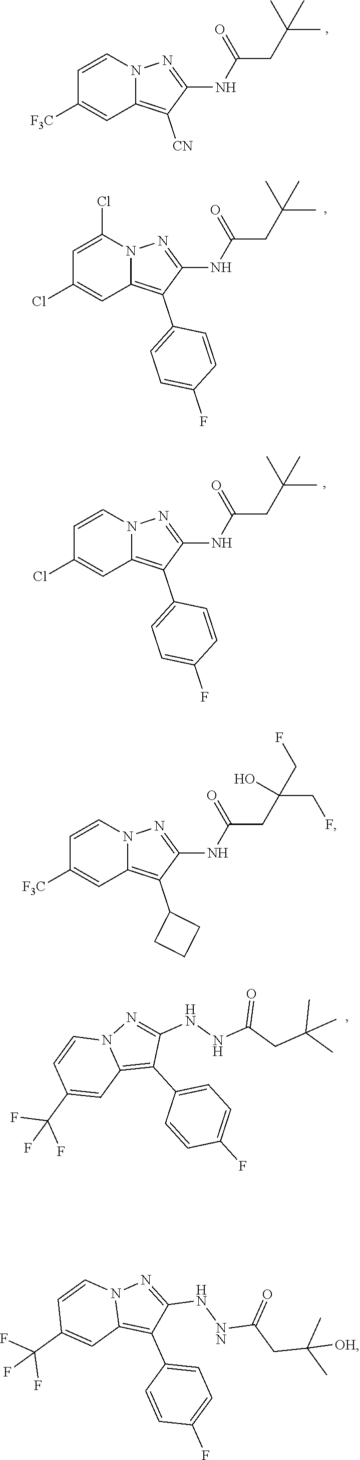

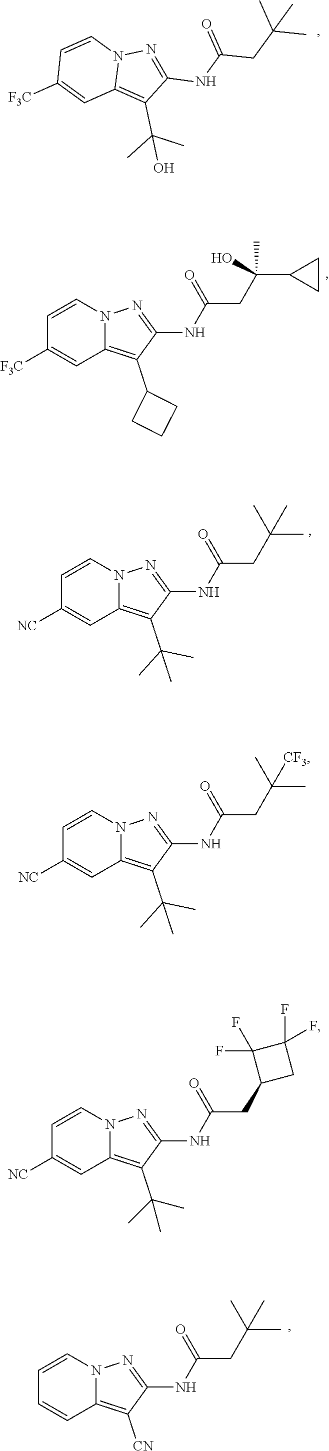 [SMILES: CC(C)(C)CC(=O)Nc1nn2ccc(C(F)(F)F)cc2c1C(C)(C)O ; C[C@@](O)(CC(=O)Nc1nn2ccc(C(F)(F)F)cc2c1C1CCC1)C1CC1 ; CC(C)(C)CC(=O)Nc1nn2ccc(C#N)cc2c1C(C)(C)C ; CC(C)(C)c1c(NC(=O)CC(C)(C)C(F)(F)F)nn2ccc(C#N)cc12 ; CC(C)(C)c1c(NC(=O)C[C@@H]2CC(F)(F)C2(F)F)nn2ccc(C#N)cc12 ; CC(C)(C)CC(=O)Nc1nn2ccccc2c1C#N]

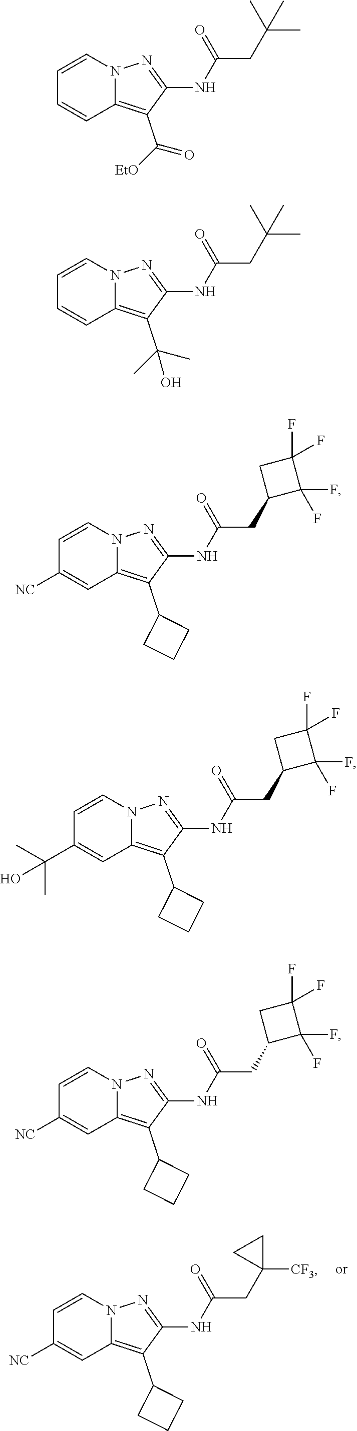

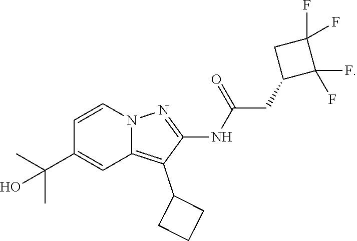 [SMILES: CC(C)(O)c1ccn2nc(NC(=O)C[C@@H]3CC(F)(F)C3(F)F)c(C3CCC3)c2c1]

16. A pharmaceutical composition comprising a compound of 1, 2, 3, 4, 5, 6, 7, 8, 9, 10, 11, 12, 13, 14, or 15, or a pharmaceutically acceptable salt thereof.

17. A method of treating a disorder associated with a Kv7 potassium channel comprising administering a therapeutically effective amount of a compound of 1, 2, 3, 4, 5, 6, 7, 8, 9, 10, 11, 12, 13, 14, or 15, or pharmaceutically acceptable salt thereof, to a subject in need thereof.

18. The method of claim 17, wherein the disorder is selected from epilepsy, epileptic spasms, neonatal spasms, neonatal seizures, benign familial neonatal epilepsy (BFNE), neonatal epileptic encephalopathy (NEE), focal seizures, focal epilepsy, myoclonic seizures, tonic and clonic seizures, tonic-clonic (grand mal) seizures, partial-onset seizures, pharmacoresistant seizures, pain, migraine, a disorder of neurotransmitter release, early infantile epileptic encephalopathy (EIEE, Ohtahara syndrome) with delayed psychomotor development, generalized tonic seizures, abnormal globus pallidus morphology, apnea, cerebral edema, a dyskinesia, dystonia, facial erythema, muscular hypotonia, febrile seizures, hypoplasia of the corpus callosum, hypsarrhythmia, focal clonic seizure, generalized tonic-clonic seizures, infantile spasms (West syndrome), Doose syndrome (myoclonic astatic epilepsy of childhood), benign Rolandic epilepsy (BRE), Rasmussen syndrome, Lennox-Gastaut syndrome, electrical status epilepticus of sleep (ESES), Sturge-Weber syndrome, juvenile myoclonic epilepsy, Dravet syndrome, myokymia, spastic tetraparesis, myokymia, mania, a hearing disorder, neuroradiological alterations, deafness, neuropathic pain, inflammatory pain, persistent pain, cancer pain, postoperative pain, brain ischemic injury, age-related impairment of memory, stress-related dysfunction of the neuroendocrine system, anxiety, depression, substance abuse, addiction, chronic alcohol intake, schizophrenia, autism, amyotrophic lateral sclerosis, Alzheimer's disease, tinnitus, and combinations thereof.

19. A method of treating a disorder associated with a KCNQ2 or a KCNQ3 mutation comprising administering a therapeutically effective amount of a compound of claim 1, 2, 3, 4, 5, 6, 7, 8, 9, 10, 11, 12, 13, 14, or 15, or pharmaceutically acceptable salt thereof, to a subject in need thereof.

20. The method of claim 19, wherein the disorder is selected from epilepsy, epileptic spasms, neonatal spasms, neonatal seizures, benign familial neonatal epilepsy (BFNE), neonatal epileptic encephalopathy (NEE), focal seizures, focal epilepsy, myoclonic seizures, tonic and clonic seizures, tonic-clonic (grand mal) seizures, partial-onset seizures, pharmacoresistant seizures, pain, migraine, a disorder of neurotransmitter release, early infantile epileptic encephalopathy (EIEE, Ohtahara syndrome) with delayed psychomotor development, generalized tonic seizures, abnormal globus pallidus morphology, apnea, cerebral edema, a dyskinesia, dystonia, facial erythema, muscular hypotonia, febrile seizures, hypoplasia of the corpus callosum, hypsarrhythmia, focal clonic seizure, generalized tonic-clonic seizures, infantile spasms (West syndrome), Doose syndrome (myoclonic astatic epilepsy of childhood), benign Rolandic epilepsy (BRE), Rasmussen syndrome, Lennox-Gastaut syndrome, electrical status epilepticus of sleep (ESES), Sturge-Weber syndrome, juvenile myoclonic epilepsy, Dravet syndrome, myokymia, spastic tetraparesis, myokymia, mania, a hearing disorder, neuroradiological alterations, deafness, neuropathic pain, inflammatory pain, persistent pain, cancer pain, postoperative pain, brain ischemic injury, age-related impairment of memory, stress-related dysfunction of the neuroendocrine system, anxiety, depression, substance abuse, addiction, chronic alcohol intake, schizophrenia, autism, amyotrophic lateral sclerosis, Alzheimer's disease, tinnitus, and combinations thereof.
